(19) United States
(12) Patent Application Publication
(10) Pub. No.: US 2009/0203708 A1
(43) Pub. Date: Aug. 13, 2009

(54) NOVEL SUBSTITUTED-1-H-QUINAZOLINE-2,4-DIONE DERIVATIVES, PREPARATION METHOD THEREOF AND PHARMACEUTICAL COMPOSITION CONTAINING THE SAME

(75) Inventors: Churlmin Seong, Taejeon-si (KR); Nosang Park, Taejeon-si (KR); Jinil Choi, Taejeon-si (KR); Chul Min Park, Taejeon-si (KR); Wookyu Park, Chungcheongbuk-do (KR); Jaeyang Kong, Taejeon-si (KR)

Correspondence Address: LUCAS & MERCANTI, LLP, 475 PARK AVENUE SOUTH, 15TH FLOOR, NEW YORK, NY, 10016, US

(73) Assignee: Korea Research Institute of Chemical Technology, Taejeon-si (KR)

(21) Appl. No.: 12/304,670

(22) PCT Filed: Jul. 5, 2006
(86) PCT No.: PCT/KR2006/002631

§ 371 Date: Dec. 12, 2008

(51) Int. Cl. A61K 31/496; C07D 403/10; A61P 25/28

(52) U.S. Cl. 514/252.17; 544/285

(57) ABSTRACT

Disclosed herein are novel substituted-1H-quinazoline-2,4-dione derivatives, a preparation method thereof, and a pharmaceutical composition containing the same. The novel substituted-1H-quinazoline-2,4-dione derivatives are excellent in binding affinity and selectivity for 5-HT6 receptors over other receptors, inhibit serotonin(5-HT)-stimulated cAMP accumulation, and disrupt apomorphine(2 mg/kg, i.p.)-induced hyperactivity in rats. Thanks to these effects, the derivatives are useful in the treatment of 5-HT6 receptor-related central nervous system diseases.

### TECHNICAL FIELD

The present invention relates to a novel substituted-1H-quinazoline-2,4-dione derivative, a method for preparing the same, and a pharmaceutical composition containing the same as an active ingredient.

### BACKGROUND ART

Although its function in the central nervous system has yet to be elucidated, 5-HT has been implicated in the etiology of various disease states, and may be particularly important in mental illnesses, such as depression, anxiety, schizophrenia, eating disorders, obsessive compulsive disorder (OCD), migraines and panic disorder. Recent great advances in pharmacology, molecular biology and genetics on the serotonin system bring the promise of a great improvement in chemical therapy for specific nerve diseases. In practice, many currently used treatments for these disorders are thought to act by modulating serotoninergic tone.

During the last decade, multiple 5-HT receptor subtypes have been characterized. Initially, receptor subtypes were characterized using pharmacological tools only. On the basis of the receptor binding profiles, common secondary messenger coupling and the functional activity of ligands, four main subgroups of 5-HT receptors, termed 5-HT1, 5-HT2, 5HT3 and 5-HT4, were identified. More recently, molecular biological techniques have both confirmed this classification, by finding that the subgroups have rather dissimilar protein structures, and led to the identification of novel 5-HT receptors (5-HT1F, 5-HT5, 5-HT6 and 5HT7) enabling them to be cloned, expressed in cultured cell lines and pharmacologically and functionally characterized [Hoyer, D. et al., Pharmacol. Biochem. Behav. 2002, 71, 533-554; Kroeze, W. K. et al, Curr. Top. Med. Chem. 2002, 2, 507-528].

More recently, a 5-HT6 has been cloned from rat cDNA on the basis of its homology with the G-protein-coupled receptors, which were previously cloned. The rat receptor consists of 438 amino acids with seven transmembrane domains, and functions to increase adenylyl cyclase activity via Gs G-protein [Monsma, F. J. et al, Mol. Pharmacol. 1993, 43, 320-327]. Human 5-HT6 receptor, 440 amino acid residues long, has been found not only to share 89% overall sequence homology with the rat receptors, but also to act similar thereto, to increase the activity of an adenylate cyclase second messenger system [Kohen, R. et al., J. Neurochem. 1996, 66, 47-56]. Both rat and human 5-HT6 mRNAs are located in the striatum, amygdale, nucleus accumbens, hippocampus, cortex, and olfactory tubercle, but neither have been found in peripheral organs.

In pharmacological studies, tritiated 5-HT, [³H] LSD, and [¹²⁵I]-2-iodo LSD have been used to radiolabel 5-HT6 receptors. 5-HT binds to 5-HT6 receptors with moderately high affinity (Ki=50˜150 nM). Tricyclic antipsychotic agents and some antidepressants bind with significant affinity. A related investigation examined antipsychotics in greater detail and found that representative members of several classes of antipsychotics bind with high affinity. Examples include phenothiazine chlorpromazine, thioxanthene, chlorprothixene, diphenylbutylpiperidine pimozide, the heterocyclic antipsychotic agent loxapine, and clozapine [Roth, B. L. et al., J. Pharmacol. Exp. Ther. 1994, 268, 1403-1410]. These results suggest that 5-HT6 receptors might play a role in certain types of psychoses and that they might represent significant targets for atypical antipsychotics in particular.

Until selective ligands were developed, the exploration of 5-HT6 pharmacology was largely dependent on the use of nonselective agents. In the absence of selective ligands for the receptor, functional studies have been carried out using an antisense approach. 5-HT6 specific antisense produced a specific behavioural syndrome of yawning, stretching and chewing, but had no other discernable action in rats. The non-selective ligands were useful for investigating the pharmacology of 5-HT6 systems in preparations where other 5-HT receptors were absent (e.g., cAMP assays), however, owing to their lack of selectivity, they were of limited value in most other pharmacological studies. The recent advent of selective agents has brought great progress to 5-HT6 studies. The development of more selective ligands may therefore lead to treatments with increased efficacy and reduced side effects. Alternatively, selective ligands may form the basis of completely novel therapies. It was not until 1998 that the first 5-HT6-selective antagonist was described, and this prompted others to quickly report their efforts in this area. Sleight et al., at Hoffman-LaRoche Co., identified the bisaryl sulfonamides Ro 04-6790 (1, Ki=55 nM) and Ro 63-0563 (2, Ki=12 nM) as very selective 5-HT6 antagonists [Sleight, A. J. et al., Br. J. Pharmacol. 1998, 124, 556-562]. Shortly thereafter, MS-245 (3, Ki=2.3 nM) was reported. Interestingly, although they were independent discoveries, all three were identified through random screening methods and all three possess a sulfonamide moiety. One problem associated with these antagonists was their low penetration into the CNS. At the time, Smith-Kline Beecham Co. also pinched out compound 4 via high-throughput screening. It displayed high affinity (Ki=5 nM) for 5-HT6 receptors, 50-fold or higher selectivity over 10 other 5-HT receptors, and no measurable affinity for 50 other receptor/binding sites. It was a pure antagonist of cAMP accumulation (pKb=7.8) [Bromidge, S. M. et al., J. Med. Chem. 1999, 42, 202-205].

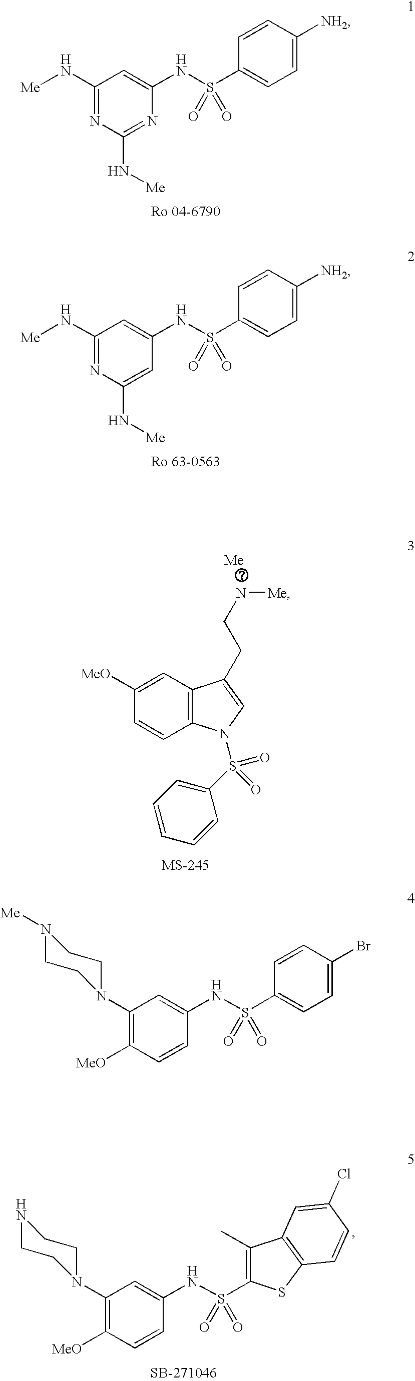

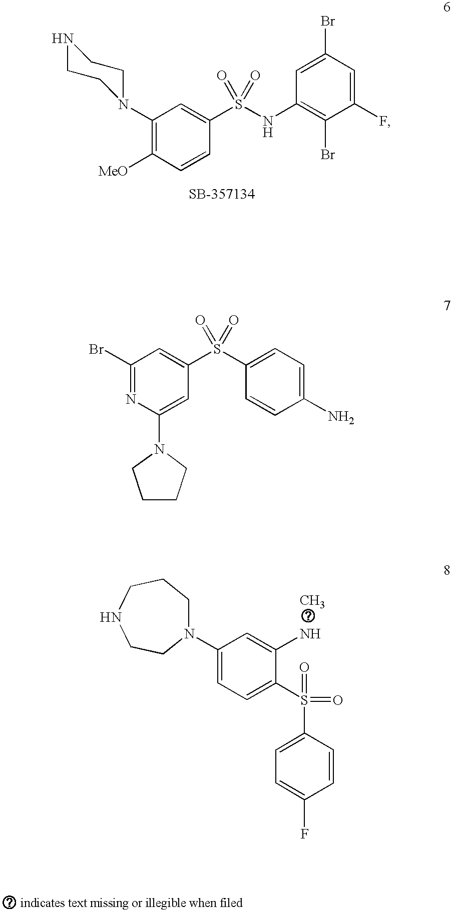

It was moderately brain penetrant (25%), but subject to rapid blood clearance, resulting in low bioavailability. An ensuing study for structure activity revealed that SB-271046 (5, Ki=1 nM; >200 selectivity over 50 other receptors) retained antagonist activity, and although low in brain penetration (10%), it showed excellent (>80%) oral bioavailability. Subsequent studies by this group led to the development of SB-357134 (6, Ki=3 nM) having a low clearance rate and excellent oral bioavailability. In 1999, Glennon et al. undertook a structure affinity investigation of the binding of tryptamine derivatives at human 5-HT6 receptors [Glennon, R. A. et al., J. Med. Chem. 2000, 43, 1011-1018]. MS-245 was found to be an antagonist (pA2=8.88) with high affinity (Ki=2.3 nM). In contrast to the above-mentioned sulfonamides or tryptamine derivatives, Hoffmann-LaRoche (7) and Pharmacia-Upjohn (8, Ki=1.4 nM) recently revealed several sulfones [Slassi, A. et al, Expert Opin. Ther. Pat. 2002, 12, 513-527]. Newer agents continue to be developed in attempts to improve pharmacokinetic and pharmacodynamic properties. Now that some tools are available, attention is focusing more and more on the function of 5-HT6 receptors.

As mentioned above, atypical antipsychotics display particularly high affinity for these receptors. In addition, the tritiated atypical antipsychotic agent [³H] clozapine was shown to label two populations of receptors in the rat brain, and one of the populations was thought to represent 5-HT6 receptors [Glatt, C. E. et al., Mol. Med. 1995, 1, 398-406]. Vogt et al. performed a systematic mutation scan of the coding region of the 5-HT6 receptor gene of 137 individuals (including schizophrenic and depressed patients) and concluded that the gene might be involved in bipolar affective disorder [Vogt, I. R. et al., Am. J. Med. Genet. 2000, 96, 217-221].

Prior to the identification of 5-HT6-selective agents, Bourson et al. demonstrated that the intracerebroventricular administration of antisense oligonucleotides into rats produced a specific behavior of yawning, stretching, and chewing, which could be antagonized by atropine [Bourson, A. et al., J. Pharmacol. Exp. Ther. 1995, 274, 173-180]. Sleight et al. demonstrated that Ro 04-6790 (1) was capable of inducing this same effect. Owing to a relationship between cholinergic function and cognition, this allowed the speculation that 5-HT6 receptors might be involved in memory and cognitive dysfunction [Sleight, A. J. et al., Neuropharmacology 2001, 41, 210-219; Rogers, D. C. et al, Psychopharmacology (Berlin) 2001, 158, 114-119]. On the basis of the fact that antisense oligonucleotide pretreatment and Ro 04-6790 administration both induced decreased food intake by rats, it was suggested that the 5-HT6 receptors might be involved in the regulation of feeding. Moreover, Russell and Dias have questioned the postulate that 5-HT6 antagonists increase cholinergic transmission [Russell, M. G. N.; Dias, R., Curr. Top. Med. Chem. 2002, 2, 643-654]. Despite the mechanistic disagreement, there is evidence for the involvement of 5-HT6 receptors in learning and memory. When a water maze was used with rats as subjects, SB-271046 (5) and SB-357134 (6) showed significant improvement in retention of a previously learned task. Furthermore, SB-271046 (5) increased extracellular glutamate levels in the frontal cortex and dorsal hippocampus by several times, indicating that the selective enhancement of excitatory neurotransmission by SB-271046 supports an important role for 5-HT6 receptor antagonists in the treatment of cognitive disorders and memory dysfunction [Dawson, L. A. et al., Neuropsychopharmacology 2001, 25, 662-668]. In addition, SB-357134 (6) produced a potent and dose-dependent increase in seizure threshold (rat maximal electroseizure threshold) following oral administration, suggesting possible therapeutic utility in convulsive disorders (Stean, T. O. et al., Pharmacol. Biochem. Behav. 2002, 71, 645-654]. These findings are consistent with an earlier finding that SB-271046 (5) and Ro 04-6790 (1) possess anticonvulsant activity.

As described above, there is a lot of evidence for the involvement of 5-HT6 in psychosis. There is still more evidence reported supporting the fact that these receptors are involved in cognition and learning and the fact that they might play a role in convulsive disorders and appetite control. Although additional studies are certainly warranted, particularly with some of the newer 5-HT6 antagonists, which are more brain-penetrant than the earlier agents, the future of 5-HT6 receptor ligands as potential therapeutic agents is quite promising.

Leading to the present invention, intensive and thorough research into 5-HT6 antagonists, conducted by the present inventors, resulted in the finding that quinazoline-2,4-dione derivatives, having neither sulfonamide nor sulfonic structures as disclosed in the prior art, can function as 5-HT6 antagonists having excellent binding strength and selectivity.

### DISCLOSURE OF THE INVENTION

It is therefore an object of the present invention to provide novel substituted-1H-quinazoline-2,4-dione derivatives which can antagonize 5-HT6 with excellent binding affinity and selectivity for 5-HT6 receptors over other receptors, and pharmaceutically acceptable salts thereof.

It is another object of the present invention to provide a method for preparing the novel substituted-1H-quinazoline-2,4-dione derivatives.

It is a further object of the present invention to provide a pharmaceutical composition for the treatment of central nervous system diseases, comprising the novel substituted-1H-quinazoline-2,4-dione derivatives, pharmaceutically acceptable salts thereof or prodrugs thereof as active ingredients.

In accordance with an aspect thereof, the present invention provides a novel substituted-1H-quinazoline-2,4-dione derivative, represented by Chemical Formula 1, and a pharmaceutically acceptable salt thereof.

In accordance with another aspect thereof, the present invention provides a method for the preparation of the novel substituted-1H-quinazoline-2,4-dione derivative.

It accordance with a further aspect thereof, the present invention provides a pharmaceutical composition for the treatment of central nervous system diseases, comprising the novel substituted-1H-quinazoline-2,4-dione derivative or a pharmaceutically acceptable salt or prodrug thereof as an active ingredient.

As will be apparent later, the novel substituted-1H-quinazoline-2,4-dione derivatives represented by Chemical Formula 1 in accordance with the present invention have excellent binding affinity and selectivity for 5-HT6 receptors over other receptors, inhibit serotonin(5-HT)-stimulated cAMP accumulation, and disrupt apomorphine (2 mg/kg, i.p.)-induced PPI, but cause no rotarod dysfunction when administered in amounts up to 300 mg/kg. Thanks to these effects, the derivatives of the present invention may be useful in the treatment of 5-HT6 receptor-related central nervous system diseases.

### BRIEF DESCRIPTION OF THE DRAWINGS

The above and other objects, features and other advantages of the present invention will be more clearly understood from the following detailed description taken in conjunction with the accompanying drawings, in which:

FIG. 1 is a graph showing the inhibitory effects of the compound according to an embodiment of the present invention and methiothepin on 5HT6 receptor-mediated cAMP accumulation in HeLa cells (: Example 40, ▾: methiothepin).

FIGS. 2 and 3 are graphs showing the effect of the compound according to the present invention on apomorphine-induced PPI disruption.

### BEST MODE FOR CARRYING OUT THE INVENTION

In order to achieve the above objects, the present invention provides a novel substituted-1H-quinazoline-2,4-dione derivative represented by the following chemical formula 1, a pharmaceutically acceptable salt thereof, a method for the preparation thereof, and therapeutic uses thereof:

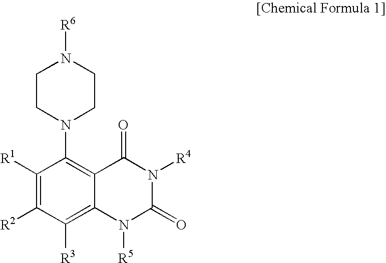

wherein,

R¹, R² and R³ are independently a hydrogen, halogen, amino, cycloamino, nitro, cyano, alkyl, haloalkyl, alkoxy, naloalkoxy, piperidinyl or N-methyl piperidinyl group;

R⁴ is a hydrogen, alkyl, cycloalkyl, haloalkyl, aryl, heteroaryl, aralkyl, heteroarylalkyl, alkoxy, aryloxy, acylamino, arylsulfonylamino, arylsulfonylureido, alkylcarboxylate, arylcarboxylate, aralkylcarhoxylate, alkylureido or arylureido group;

R⁵ is a hydrogen, alkyl, cycloalkyl, haloalkyl, aryl, arylalkyl, heteroaryl or heteroarylalkyl group; and

R⁶ is a hydrogen, alkyl or aryl group.

The term “alkyl”, as used herein, is intended to refer to a straight or branched chain containing 1 to 12 carbon atoms, and may be typified by methyl, ethyl, propyl, isopropyl, butyl, sec-butyl, tert-butyl, pentyl, hexyl, cyclopropylmethyl, cyclohexylmethyl, octyl, decyl, and dodecyl.

The term “cycloalkyl”, as used herein, is intended to refer to a cyclic carbon ring containing 3 to 10 carbon atoms, and may be typified by cyclopropyl, cyclobutyl, cyclopentyl, cyclohexyl and cycloheptyl.

The term “alkoxy”, as used herein, is intended to refer to an alkoxy group containing 1 to 7 carbon atoms, and may be typified by methoxy, ethoxy, propyloxy, isopropyloxy, butoxy, sec-butoxy, tert-butoxy, pentoxy, hexyloxy and cyclohexylmethoxy.

As used herein, the term “haloalkyl” is intended to refer to an alkyl group having at least one fluoro- or chloro-substituent, and may be typified by fluoromethyl, difluoromethyl, trifluoromethyl, pentafluoroethyl, 1,1-difluoroethyl and trichloromethyl.

As used herein, the term “aryl” is intended to refer to a cyclic carbon compound with aromaticity and may be typified by phenyl, naphthyl, phenanthryl, anthracyl, indenyl, biphenyl, and fluorenyl.

The term “heteroaryl”, as used herein, is intended to refer to an aryl group having 1 to 4 substituents selected from among O, N, S and combinations thereof, and may be typified by pyridyl, quinolinyl, isoquinolinyl, pyridazinyl, pyrimidinyl, pyrazinyl, pyrrolyl, indolyl, pyranyl, furyl, benzimidazolyl, benzofuryl, thienyl, benzothienyl, imidazolyl, oxadiazolyl, thiazolyl, and thiadiazolyl.

The aryl group and the heteroaryl group are independently or optionally substituted with 1 to 3 substituents selected from among halogen, nitro, amino, cyano, cycloamino, hydroxy, carboxyl, thiol, alkyl, aryl, heteroalkyl, heteroaryl, alkoxy, aryloxy, acyloxy, acylamino, arylsulfonylamino, arylsulfonylureido, heteroaryl, alkylthio, arylthio, alkylcarboxylate, arylcarboxylate, aralkylcarboxylate, alkylureido, arylureido, alkylamidino, arylamidino, and combinations thereof.

The term “heteroarylalkyl” is intended to refer to an alkyl group comprising the above-mentioned “heteroaryl”. Likewise, the term “arylalkyl” is intended to refer to an alkyl group comprising the above-mentioned aryl.

The term “amino”, as used herein, is intended to refer to NH₂, NHR⁷ or NR⁷R⁸, in which R⁷ and R⁸ are each independently a C₁˜C₄ alkyl. The “cycloamino” includes a bicyclic amino group, such as aziridinyl, azetidinyl, pyrrolidinyl, piperidinyl, morpholinyl, azepanyl, diazepanyl, 1,4-diazepan-1-yl, octahydro-pyrrolo[3,4-b]pyridin-6-yl, 1,4-diazabicyclo[4.3.0]non-4-yl, 1,4-diazabicyclo[4.4.0]dex-4-yl, 1,4-diazabicyclo[3.3.1]non-4-yl, 1,4-diazabicyclo[3.2.1]oct-4-yl or hexahydropyrrolo[3,4-c]pyro-2-yl.

The halogen atom includes a fluorine, chlorine, bromine or iodine atom.

Preferably, R¹, R² and R³ are independently or optionally one selected from among hydrogen, chloro, bromo or methoxy,

R⁴ is hydrogen; an unsubstituted or C₅˜C₁₀ aryl-substituted straight or branched alkyl of C₁˜C₁₀; cycloalkyl of C₅˜C₁₀; an unsubstituted or C₁˜C₃ alkoxy-substituted aryl of C₅˜C₁₀; an arylalkyl of C₅˜C₁₂, substituted or not substituted with a substituent selected from among halogen, straight or branched alkyl of C₁˜C₅, alkoxy of C₁˜C₅, hydroxy, amino, nitro, cyano, C₁˜C₃ alkoxycarbonyl and combinations thereof; or heteroaryl or heteroarylalkyl of C₅˜C₁₀ [symbol] containing at least one heteroatom selected from among N, O and S,

R⁵ is hydrogen, a straight or branched alkyl of C₁˜C₅, or an arylalkyl of C₅˜C₁₂, substituted or not substituted with a substituent selected from among halogen, a straight or branched alkyl of C₁˜C₅, an alkoxy of C₁˜C₅, hydroxy, an amino, a nitro, a cyano, an alkoxycarbonyl of C₁˜C₃ and combinations thereof, and

R⁶ is hydrogen, or a straight or branched alkyl of C₁˜C₅.

More preferably, R⁴ is methyl, ethyl, propyl, n-butyl, 3-methylbutyl, cyclohexylmethyl, octyl, phenyl, methoxyphenyl, benzyl, fluorobenzyl, bromobenzyl, chlorobenzyl, iodobenzyl, methylbenzyl, methoxybenzyl, hydroxybenzyl, nitrobenzyl, aminobenzyl, cyanobenzyl, methoxycarbonylbenzyl, dimethylbenzyl, (R)-1-phenylethyl, (S)-1-phenylethyl, phenethyl, phenylpropyl, dimethylphenylpropyl, isobutylphenylpropyl, naphthalenylmethyl, methylfuranylmethyl or pyrimidylmethyl,

R⁵ is hydrogen, methyl, ethyl, propyl, n-butyl or benzyl, and

R⁶ is hydrogen or methyl.

The salts of the compound represented by Chemical Formula 1 are nontoxic enough to be pharmaceutically acceptable. However, other salts, even if toxic, may be used if pharmaceutically acceptable.

Examples of such pharmaceutically acceptable salts of the compound of Chemical Formula 1 include alkali metal salts such as lithium, sodium or potassium salts; alkaline earth metal salts such as calcium or magnesium salts; and salts formed with suitable organic ligands, such as quaternary ammonium salts. In the case of acid addition salt, for example, a solution of the compound according to the present invention may be mixed with a pharmaceutically acceptable non-toxic acid solution such as hydrochloric acid, fumaric acid, maleic acid, succinic acid, acetic acid, citric acid, tartaric acid, carbonic acid or phosphoric acid.

The compounds according to the present invention include prodrugs of the compounds of formula 1. Generally, such prodrugs will be functional derivatives of the compounds of formula 1 which are readily converted in vivo into the required compounds. The suitable prodrugs according to the present invention may be selected and prepared using a conventional method (“Design of Prodrugs”, ed. H. Bundgaard, Elsevier, 1985).

In addition, the compound of the present invention may include any of the tautomers of the compound represented by chemical formula 1.

Further, in the case in which the compounds of formula 1 according to the present invention have at least one asymmetric center, they may exist accordingly as enantiomers. In the case in which the compounds of formula 1 according to the present invention have two or more asymmetric centers, they may exist additionally as diastereomers. All of the isomers of the compound according to the present invention and mixtures thereof fall within the scope of the present invention.

More preferable examples of the compound of Chemical Formula 1 according to the present invention include the following compounds, pharmaceutically acceptable salts and prodrugs thereof, but are not limited thereto:
(1) 1-benzyl-7-chloro-3-methyl-5-(4-methyl-piperazin-1-yl)-1H-quinazoline-2,4-dione;
(2) 1-benzyl-7-chloro-3-ethyl-5-(4-methyl-piperazin-1-yl)-1H-quinazoline-2,4-dione;
(3) 1-benzyl-7-chloro-5-(4-methyl-piperazin-1-yl)-3-propyl-1H-quinazoline-2,4-dione;
(4) 1-benzyl-3-butyl-7-chloro-5-(4-methyl-piperazin-1-yl)-1H-quinazoline-2,4-dione;
(5) 1-benzyl-7-chloro-3-(3-methyl-butyl)-5-(4-methyl-piperazin-1-yl)-1H-quinazoline-2,4-dione;
(6) 1-benzyl-7-chloro-3-cyclohexylmethyl-5-(4-methyl-piperazin-1-yl)-1H-quinazoline-2,4-dione;
(7) 1-benzyl-7-chloro-5-(4-methyl-piperazin-1-yl)-3-octyl-1H-quinazoline-2,4-dione;
(8) 1-benzyl-7-chloro-5-(4-methyl-piperazin-1-yl)-3-octyl-1H-quinazoline-2,4-dione;
(9) 1-benzyl-7-chloro-3-(4-methoxy-phenyl)-5-(4-methyl-piperazin-1-yl)-1H-quinazoline-2,4-dione; quinazoline-2,4-dione;
(10) 1,3-dibenzyl-7-chloro-5-(4-methyl-piperazin-1-yl)-1H-quinazoline-2,4-dione;
(11) 1-benzyl-7-chloro-3-(2-fluoro-benzyl)-5-(4-methyl-piperazin-1-yl)-1H-quinazoline-2,4-dione;
(12) 1-benzyl-7-chloro-3-(3-fluoro-benzyl)-5-(4-methyl-piperazin-1-yl)-1H-quinazoline-2,4-dione;
(13) 1-benzyl-7-chloro-3-(4-fluoro-benzyl)-5-(4-methyl-piperazin-1-yl)-1H-quinazoline-2,4-dione;
(14) 1-benzyl-3-(2-bromo-benzyl)-7-chloro-5-(4-methyl-piperazin-1-yl)-1H quinazoline-2,4-dione;
(15) 1-benzyl-3-(3-bromo-benzyl)-7-chloro-5-(4-methyl-piperazin-1-yl)-1H-quinazoline-2,4-dione;
(16) 1-benzyl-3-(4-bromo-benzyl)-7-chloro-5-(4-methyl-piperazin-1-yl)-1H-quinazoline-2,4-dione;
(17) 1-benzyl-7-chloro-3-(2-chloro-benzyl)-5-(4-methyl-piperazin-1-yl)-1H-quinazoline-2,4-dione;
(18) 1-benzyl-7-chloro-3-(3-chloro-benzyl)-5-(4-methyl-piperazin-1-yl)-1H-quinazoline-2,4-dione;
(19) 1-benzyl-7-chloro-3-(4-chloro-benzyl)-5-(4-methyl-piperazin-1-yl)-1H-quinazoline-2,4-dione;
(20) 1-benzyl-7-chloro-3-(3-iodo-benzyl)-5-(4-methyl-piperazin-1-yl)-1H-quinazoline-2,4-dione;
(21) 1-benzyl-7-chloro-3-(4-iodo-benzyl)-5-(4-methyl-piperazin-1-yl)-1H-quinazoline-2,4-dione;
(22) 1-benzyl-7-chloro-3-(2-methyl-benzyl)-5-(4-methyl-piperazin-1-yl)-1H-quinazoline-2,4-dione;
(23) 1-benzyl-7-chloro-3-(3-methyl-benzyl)-5-(4-methyl-piperazin-1-yl)-1H-quinazoline-2,4-dione;
(24) 1-benzyl-7-chloro-3-(4-methyl-benzyl)-5-(4-methyl-piperazin-1-yl)-1H-quinazoline-2,4-dione;
(25) 1-benzyl-7-chloro-3-(2-methoxy-benzyl)-5-(4-methyl-piperazin-1-yl)-1H-quinazoline-2,4-dione;
(26) 1-benzyl-7-chloro-3-(3-methyl-benzyl)-5-(4-methyl-piperazin-1-yl)-1H-quinazoline-2,4-dione;
(27) 1-benzyl-7-chloro-3-(4-methoxy-benzyl)-5-(4-methyl-piperazin-1-yl)-1H-quinazoline-2,4-dione;
(28) 1-benzyl-7-chloro-3-(2-hydroxy-benzyl)-5-(4-methyl-piperazin-1-yl)-1H-quinazoline-2,4-dione;
(29) 1-benzyl-7-chloro-3-(3-hydroxy-benzyl)-5-(4-methyl-piperazin-1-yl)-1H-quinazoline-2,4-dione;
(30) 1-benzyl-7-chloro-3-(4-hydroxy-benzyl)-5-(4-methyl-piperazin-1-yl)-1H-quinazoline-2,4-dione;
(31) 1-benzyl-7-chloro-5-(4-methyl-piperazin-1-yl)-3-(2-nitro-benzyl)-1H-quinazoline-2,4-dione;
(32) 1-benzyl-7-chloro-5-(4-methyl-piperazin-1-yl)-3-(3-nitro-benzyl)-1H-quinazoline-2,4-dione;
(33) 1-benzyl-7-chloro-5-(4-methyl-piperazin-1-yl)-3-(4-nitro-benzyl)-1H-quinazoline-2,4-dione;
(34) 3-(2-amino-benzyl)-1-benzyl-7-chloro-5-(4-methyl-piperazin-1-yl)-1H-quinazoline-2,4-dione;
(35) 3-(3-amino-benzyl)-1-benzyl-7-chloro-5-(4-methyl-piperazin-1-yl)-1H-quinazoline-2,4-dione;
(36) 3-(4-amino-benzyl)-1-benzyl-7-chloro-5-(4-methyl-piperazin-1-yl)-1H-quinazoline-2,4-dione;
(37) 4-[1-benzyl-7-chloro-5-(4-methyl-piperazin-1-yl)-2,4-dioxo-1,4-dihydro-2H-quinazoline-3-ylmethyl]-benzonitrile;
(38) 4-[1-benzyl-7-chloro-5-(4-methyl-piperazin-1-yl)-2,4-dioxo-1,4-dihydro-2H-quinazoline-3-ylmethyl]-benzoic acid methyl ester;
(39) 1-benzyl-7-chloro-3-(3,4-dimethyl-benzyl)-5-(4-methyl-piperazin-1-yl)-1H-quinazoline-2,4-dione;
(40) 1-benzyl-7-chloro-5-(4-methyl-piperazin-1yl)-3-[(R)-1-phenyl-e % l]-1H-quinazoline-2,4-dione;
(41) 1-benzyl-7-chloro-5-(4-methyl-piperazin-1-yl)-3-[(S)-1-phenyl-ethyl]-1H-quinazoline-2,4-dione;
(42) 1-benzyl-7-chloro-5-(4-methyl-piperazin-1-yl)-3-phenethyl-1H-quinazoline-2,4-dione;
(43) 1-benzyl-7-chloro-5-(4-methyl-piperazin-1-yl)-3-(3-phenyl-propyl)-1H-quinazoline-2,4-dione;
(44) 1-benzyl-7-chloro-3-[3-(3,5-dimethyl-phenyl)-propyl]-5-(4-methyl-piperazin-1-yl)-1H-quinazoline-2,4-dione;
(45) 1-benzyl-7-chloro-3-[3-(3-isobutyl-phenyl)-propyl]-5-(4-methyl-piperazin-1-yl)-1H-quinazoline-2,4-dione;
(46) 1-benzyl-7-chloro-5-(4-methyl-piperazin-1-yl)-3-naphthalen-1-ylmethyl-1H-quinazoline-2,4-dione;
(47) 1-benzyl-7-chloro-3-(5-methyl-furan-2-ylmethyl)-5-(4-methyl-piperazin-1-yl)-1H quinazoline-2,4-dione;
(48) 1-benzyl-7-chloro-5-(4-methyl-piperazin-1-yl)-3-pyridin-4-ylmethyl-1H-quinazoline-2,4-dione;
(49) 3-benzyl-7-chloro-5-(4-methyl-piperazin-1-yl)-1H-quinazoline-2,4-dione;
(50) 3-benzyl-7-chloro-1-methyl-5-(4-methyl-piperazin-1-yl)-1H-quinazoline-2,4-dione;
(51) 3-benzyl-7-chloro-1-ethyl-5-(4-methyl-piperazin-1-yl)-1H-quinazoline-2,4-dione;
(52) 3-benzyl-7-chloro-5-(4-methyl-piperazin-1-yl)-1-propyl-1H-quinazoline-2,4-dione;
(53) 3-benzyl-1-butyl-7-chloro-5-(4-methyl-piperazin-1-yl)-1H-quinazoline-2,4-dione;
(54) 1,3-dibenzyl-7-bromo-5-(4-methyl-piperazin-1-yl)-1H-quinazoline-2,4-dione;
(55) 1-benzyl-7-bromo-3-(2-chloro-benzyl)-5-(4-methyl-piperazin-1-yl)-1H-quinazoline-2,4-dione;
(56) 1-benzyl-7-chloro-3-(4-methyl-phenyl)-5-piperazin-1-yl-1H-quinazoline-2,4-dione;
(57) 1,3-dibenzyl-7-chloro-5-piperazin-1-yl-1H-quinazoline-2,4-dione;
(58) 1-benzyl-7-chloro-3-(2-fluoro-benzyl)-5-piperazin-1-yl-1H-quinazoline-2,4-dione;
(59) 1-benzyl-7-chloro-3-(2-chloro-benzyl)-5-piperazin-1-yl-1H-quinazoline-2,4-dione;
(60) 1-benzyl-3-(2-bromo-benzyl)-7-chloro-5-piperazin-1-yl-1H-quinazoline-2,4-dione;
(61) 1-benzyl-5-chloro-3-(3-iodo-benzyl)-7-piperazin-1-yl-1H-quinazoline-2,4-dione;
(62) 1-benzyl-5-chloro-3-(2-methyl-benzyl)-7-piperazin-1-yl-1H-quinazoline-2,4-dione;
(63) 1-benzyl-7-chloro-3-(2-methoxy-benzyl)-5-piperazin-1-yl-1H-quinazoline-2,4-dione;
(64) 1-benzyl-7-chloro-3-(3-methyl-benzyl)-5-piperazin-1-yl-1H-quinazoline-2,4-dione;
(65) 1-benzyl-7-chloro-3-(2-hydroxy-benzyl)-5-piperazin-1-yl-1H-quinazoline-2,4-dione;
(66) 1-benzyl-7-chloro-3-(2-nitro-benzyl)-5-piperazin-1-yl-1H-quinazoline-2,4-dione;
(67) 3-(2-amino-benzyl)-1-benzyl-7-chloro-5-piperazin-1-yl-1H-quinazoline-2,4-dione;
(68) 1-benzyl-7-chloro-3-[(R)-1-phenyl-ethyl]-5-piperazin-1-yl-1H-quinazoline-2,4-dione;
(69) 1-benzyl-7-chloro-3-[(S)-1-phenyl-ethyl]-5-piperazin-1-yl-1H-quinazoline-2,4-dione;
(70) 1-benzyl-7-chloro-3-phenethyl-5-piperazin-1-yl-1H-quinazoline-2,4-dione; and
(71) 1-benzyl-7-chloro-3-(3-phenyl-propyl)-5-piperazin-1-yl-1H-quinazoline-2,4-dione.

In accordance with another aspect thereof, the present invention provides a method for preparing the novel substituted-1H-quinazoline-2,4-dione derivative, as delineated in the following Reaction Scheme 1, comprising:

(a) reacting an anthranilic anhydride of Chemical Formula 2 with an amine compound of Chemical Formula 2 to obtain an intermediate I;

(b) cyclizing the intermediate I of step (a) into an intermediate II;

(c) reacting the intermediate II of step (b) with a compound of Chemical Formula 4 to obtain an intermediate III; and

(d) reacting the intermediate III of step (c) with an amine compound to produce a substituted-1H-quinazoline-2,4-dione derivative of Chemical Formula 1.

Optionally, the method may comprise modifying the substituent R¹-, R²-, R³-, R⁴-, R⁵- or R⁶- of the intermediate III or Chemical Formula 1 into a predetermined functional group subsequent to step (d). In the case of the substituent R⁴-, for example, methoxy may be converted into hydroxyl using boron tribromide, nitro may be reduced into amino in the presence of tin (II) hydride in a protic solvent under a flux condition, or a hydrogen addition reaction may be performed in the presence of a palladium catalyst.

With reference to the following Reaction Scheme 1, the preparation method of the present invention is explained in detail below.

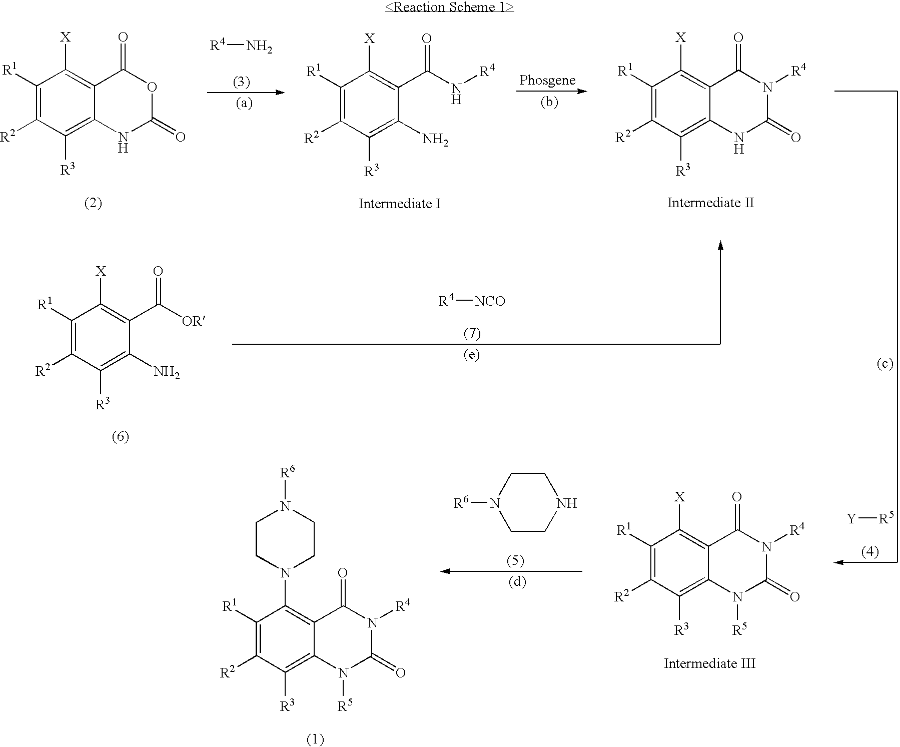

(wherein, R¹˜R⁶ are as defined in Chemical Formula 1, X is a fluorine, chlorine, bromine or iodine atom, or trifluoroacetate, Y is chloro, bromo, iodo, methanesulfonate or p-toluenesulfonate.)

In step (a), the anthranilic anhydride of Chemical Formula 2 is reacted with an amine compound of Chemical Formula 3 to give intermediate I.

The anthranilic anhydride of Chemical Formula 2, serving as a starting material in the present invention, is not commercially available, but can be prepared using a method described herein or well known in the art.

The compound of Chemical Formula 3, which is to react with the anthranilic anhydride (2), is an amine compound having R⁴ as a substituent, and preferably a primary amine. A suitable solvent containing the compound of Chemical Formula 3 and the starting material (2) is heated in the presence of a base with flux to produce open intermediate I as a result of nucleophilic substitution and decarboxylation.

Suitable for this reaction are an inert solvent, such as 1,4-dioxane or tetrahydrofuran, and a strong base, such as triethylamine or pyridine.

Next, step (b) is adapted for the cyclization of the intermediate I of step (a) into the intermediate II.

The cyclization of the intermediate I is performed with a phosgene compound, such as diphosgene or triphosgene.

An inert solvent, such as 1,4-dioxane or tetrahydrofuran, is preferable as a solvent for cyclization. This cyclization into the intermediate II may be achieved in a heating flux condition.

Alternatively, the intermediate II may be prepared from a compound of Chemical Formula 6 through cyclization with an isocyanate compound of Chemical Formula 7, as illustrated in step (e) of Reaction Scheme 1.

Subsequently, step (c) is directed to the reaction of the intermediate II of step (b) with a compound of Chemical Formula 4 into the intermediate III.

In step (c), a substituent R⁵ is introduced at N(1) of intermediate II. This substitution may be achieved at room temperature in an aprotic solvent, such as acetonitrile, tetrahydrofuran, or N,N-dimethylformamide in the presence of a base such as sodium carbonate, potassium carbonate, or sodium hydride. In the compound of Chemical Formula 4, R⁵ and Y are independently as defined in Chemical Formula 1 and Reaction Scheme 1.

Afterwards, step (d) is adapted for the reaction of an intermediate III of step (c) with an amine compound of Chemical Formula 5 into the substituted-1H-quinazoline-2,4-dione derivative of Chemical Formula 1.

In greater detail, a piperidinyl group having an R⁶ substituent is introduced onto C(5) of intermediate III. The introduction of the piperidinyl group may be achieved through a nucleophilic reaction between intermediate III and a suitable amine. The suitable amine, represented by Chemical Formula 5, is exemplified by piperazine or N-methylpiperazine. The nucleophilic substitution is preferably performed in the presence of a base, such as sodium carbonate, potassium carbonate or methylamine, in an alkaline solvent such as pyridine, in an aprotic solvent such as acetonitrile or N,N-dimethylformamide, or in a neat condition of flux temperature.

Where the above-described processes for the preparation of the compounds according to the invention give rise to mixtures of stereoisomers, these isomers may be separated through a conventional technique, such as preparative chromatography. The compounds may be prepared in racemic forms, or individual enantiomers may be prepared either through asymmetric synthesis or resolution. The compounds may, for example, be resolved into their component enantiomers by standard techniques, such as the formation of diastereomeric pairs by salt formation with an optically active acid, such as (−)-di-p-toluoyl-d-tartaric acid and/or (+)-di-p-toluoyl-1-tartaric acid, followed by fractional crystallization and regeneration of the free base. The compounds may also be resolved by formation of diastereomeric esters or amides, followed by chromatographic separation and removal of the chiral auxiliary. Therefore, all structural and optical isomers of the various compound derivatives, as well as racemic mixtures thereof, fall within the scope of the present invention.

In accordance with a further aspect thereof, the present invention provides a pharmaceutical composition of a 5-HT6 antagonistic, a pharmaceutical composition comprising the compound of formula 1 and pharmaceutically acceptable salts thereof.

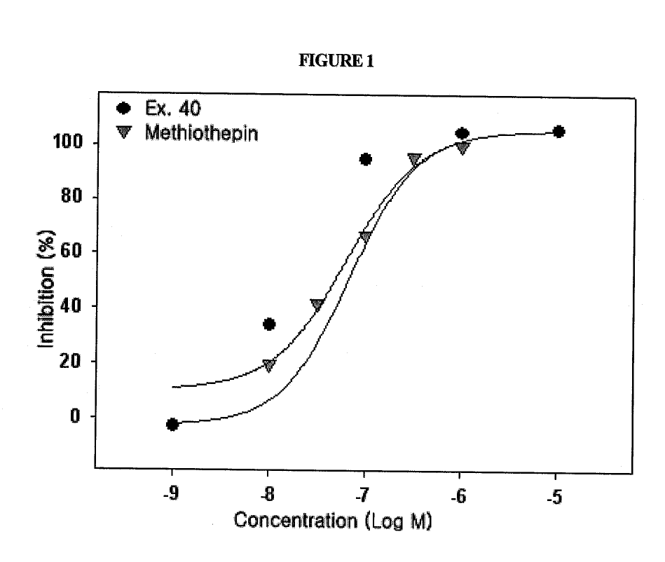

The compound of the present invention is found to have excellent binding affinity for serotonin 5-HT6 receptors (Table 3) and excellent selectivity for 5-HT6 receptors over other receptors (Table 5), inhibit intracellular serotonin(5-HT)-stimulated cAMP accumulation (FIG. 1), suppress methamphetamine (2 mg/kg, i.p.)-induced hyperactivity of rats, and avoid rotarod dysfunction at doses of less than 400 mg/kg (Table 7). Therefore, the compound of the present invention may be effective as a 5-HT6 antagonist.

Since 5-HT6 receptors are known to positively bind to an adenyl cyclase system, the effectors thereof can distinctively increase intracellular cAMP levels. Hence, materials which act to inhibit the serotonin (5-HT)-stimulated cAMP accumulation in cells can be regarded as 5-HT6 antagonists.

A prepulse inhibition experiment of acoustic startle in animals, which is adapted to examine whether a compound suppresses hyperactivity in rats, is one of the most extensively studied behavior models. A phenomenon in which, as a main startle stimulus is gradually weakened, the startle amplitude is reduced or terminated, is called prepulse inhibition (hereinafter referred to as “PPI”) (Graham, 1975). It was reported that PPI deficit emerges in patients with schizophrenia or psychosis [Braff et al, 1992; Simons and Giardina, 1992].

Therefore, the pharmaceutical composition of the present invention is applicable to the treatment of 5-HT6 receptor-mediated diseases of the central nervous system, and is particularly useful in the treatment of cognitive disorders, Alzheimer's disease, anxiety, depression, schizophrenia, stress disorder, panic disorder, phobic disorder, obsessive compulsive disorder (OCD), post-traumatic-stress syndrome, immunosuppression, psychosis, immunity decrease, mental illness, paraphrenia, mania, compulsive disorder, migraine, drug addiction, alcohol addiction, obesity, eating disorders, and sleep disorder.

The compound of the present invention may be formulated into various dosage forms, such as oral or parenteral administrations, or may be preferably administered through intravenous infusion. For pharmaceutical preparations, excipients and diluents, such as a filler, a thickening agent, a binding agent, a wetting agent, a disintegrant, and a surfactant, may generally be employed. The pharmaceutical compositions of the present invention are preferably in unit dosage forms, such as tablets, pills, capsules, powders, granules, sterile solutions or suspensions, or suppositories, for oral, intravenous, parenteral or rectal administration. For the preparation of solid compositions such as tablets, the active ingredient may be mixed with a pharmaceutical carrier, such as starch, sucrose, lactose, talc, sorbitol, stearic acid, magnesium stearate, dicalcium phosphate or gum, or a diluent such as water. In this way, a solid preformulation composition comprising a homogeneous mixture of a compound of the present invention, or a non-toxic pharmaceutically acceptable salt thereof, can be obtained. When referring to these preformulation compositions as homogeneous, it is meant that the active ingredient is dispersed evenly throughout the composition, so that the composition may be readily subdivided into equally effective unit dosage forms. This solid preformulation composition is then subdivided into unit dosage forms of the type described above, containing about 0.1 to 500 mg of the active ingredient of the present invention. The tablets or pills of the novel composition can be coated or otherwise compounded to provide a dosage form affording the advantage of prolonged action. For example, the tablet or pill can comprise an inner dosage and an outer dosage component, the latter being in the form of an envelope surrounding the former. The two components can be separated by an enteric layer which serves to resist disintegration in the stomach and permits the inner component to pass intact into the duodenum or to be delayed in release. A variety of materials can be used for such enteric layers or coatings, such materials including a number of polymeric acids and mixtures of polymeric acids with such materials as shellac, cetyl alcohol and cellulose acetate. The liquid forms in which the novel compositions of the present invention may be incorporated for administration, either orally or by injection, include aqueous solutions, syrups, aqueous or oil suspensions, and emulsions with edible oils such as cottonseed oil, sesame oil, coconut oil or peanut oil, as well as elixir and similar pharmaceutical vehicles. Suitable dispersing or suspending agents for aqueous suspensions include synthetic and natural gums such as tragacanth, acacia, alginate, dextran, sodium carboxymethylcellulose, methylcellulose, polyvinylpyrrolidone or gelatin.

The compound of the present invention in a pharmaceutical composition may be administered in a daily dose from about 0.01 to 250 mg/kg per day, preferably from about 0.05 to 100 mg/kg per day, and more preferably from about 0.05 to 5 mg/kg per day. The pharmaceutical composition of the present invention may be administered at a frequency of 1 to 4 times per day.

A better understanding of the present invention may be obtained through the following examples which are set forth to illustrate, but are not to be construed as the limit of the present invention.

### Preparation Example 1

### Preparation of 5,7-Dichloroanthranilic Anhydride

### Step 1

### Preparation of #-(3,5-dichlorophenyl)-2-hydroxyimino-acetamide

To a solution of 3,5-dichloroanilin (10.0 g, 61.7 mmol) in purified water were added conc. hydrochloric acid (12 ml) and 1,4-dioxane (20 ml), and the resulting mixture was heated until it became transparent, followed by the addition of a 50° C. solution of chloral hydrate (10.5 g, 66.9 mmol) and Na₂SO₄ (66.0 g) in purified water (224 ml) thereto. Then, the resulting mixture was added to a solution of hydroxylamine hydrochloride (13.0 g, 180 mmol) in water (60 ml) and heated for 50 min with flux. After the mixture was cooled to room temperature, the insoluble solid was filtered, washed with distilled water, and dried in a vacuum to afford the title compound as a pale solid (12.8 g, 89%).

TLC R_{f}=0.5 (ethylacetate:n-hexane=1:3);

m.p. 196-197° C.;

¹H NMR (DMSO-d₆) δ 7.39 (t, 1H, J=1.8 Hz, ArH), 7.70 (s, 1H, CHNOH), 7.89 (d, 2H, J=1.8 Hz, ArH), 10.54 (br s, 1H, NH), 12.40 (br s, 1H, NOH);

MS (EI) m/e 233 [M⁺], 216, 202, 189, 161.

Step 2

### Preparation of 4,6-dichloro-1H-indole-2,3-dione

N-(3,5-dichlorophenyl)-hydroxyimino-acetamide (10.0 g, 42.9 mmol), prepared in step 1, was slowly added to cone, sulfuric acid (50 ml) in an ice bath. At this time, the reaction mixture was required to be maintained at 50° C. or less. Following completion of the addition, the turbid solution was heated to 90° C. for 1 hour with stirring. The resulting reaction mixture was cooled to room temperature and then added to 10 volumes of crushed ice blocks and stirred for 1 hour. The insoluble solid thus formed was harvested, washed with distilled water and dried in a vacuum to give the title compound as an orange solid (8.90 g, 96%).

TLC R_{f}=0.4 (ethylacetate:n-hexane=1:3);

mp 228-230° C.;

¹H NMR (DMSO-d₆) δ 6.97 (d, 1H, J=1.8 Hz, ArH), 7.32 (d, 1H, J=1.8 Hz, ArH), 11.42 (br s, 1H, NH);

MS (EI) m/e 216 [M+], 188 [M⁺-CO₂], 160.

### Step 3

### Preparation of 2-amino-4,6-dichlorobenzoic acid

To a solution of 4,6-dihloro-1H-indole-2,3-dione (5.0 g, 23.1 mmol) in 75 ml of 1 N NaOH was partially added hydrogen peroxide (28% v/v, 10 ml) at room temperature. After stirring the mixture for 2 hours, an insoluble dark brown solid was removed by filtration. The filtrate was acidified with a cone, hydrochloric acid to a pH of 2. The yellow precipitate thus formed was harvested, washed with distilled water, and dried in a vacuum. Recrystallization in benzene produced the title compound as an ivory solid (3.90 g, 82%).

TLC R_{f}=0.1 (ethylacetate: n-hexane=1:1);

m.p. 188-189° C.;

¹H NMR (DMSO-d₆) δ 6.76 (d, 1H, J=1.9 Hz, ArH), 6.85 (d, 1H, J=1.9 Hz, ArH);

MS (EI) m/e 206 [M+], 162 [M⁺-CO₂].

### Step 4

### Preparation of 5,7-dichloroanthranilic Anhydride

A solution of the anthranilic acid (5.85 g, 28.3 mmol) and diphosgene (3.73 g, 18.9 mmol) in 70 ml of 1,4-dioxane was heated for 2 hours with flux. The reaction mixture was cooled to room temperature. The precipitate thus formed was filtered and washed several times with n-hexane. Drying in a vacuum gave the title compound as a light yellow solid (5.25 g, 80%).

¹H NMR (DMSO-4) δ 7.19 (d, 1H, J=1.9 Hz, ArH), 7.56 (d, 1H, J=1.9 Hz, ArH), 12.0 (br s, 1H, NH);

MS (EI) m/e 232 [M⁺], 188, 162.

### Preparation Example 2

### Preparation of Intermediate I

### Preparation Example 2-1

### Preparation of 2-amino-4,6-dichloro-N-methyl-benzamide (Intermediate I-1)

A solution of 5,7-dichloroanthranilic anhydride (2.00 mmol), methylamine (2.40 mmol) and TEA (2.40 mmol) in tetrahydrofuran or 1,4-dioxane (30 ml) was heated for 2 hours with flux. After the starting material anhydride disappeared, the solvent was removed through vacuum drying. The residue was washed with 1 N hydrochloric acid (100 ml). Recrystallization from a solvent containing methanol, ethylacetate or benzene for corresponding N-substituted benzamide yielded the title compound as a bright brown solid (62%).

m.p. 159-162° C.;

¹H NMR (200 MHz, CDCl₃+CD₃OD) δ 2.95 (d, 3H, J=4.9 Hz, NHCH₃), 6.63 (d, 1H, J=2.0 Hz, ArH), 6.71 (d, 1H, J=2.0 Hz, ArH), 7.12 (br s, 1H, NH);

MS (EI) m/e 219 [M⁺], 188, 148, 133, 73.

### Preparation Example 2-2

### Preparation of 2-amino-4,6-dichloro-N-ethyl-benzamide (Intermediate I-2)

The same procedure as in Preparation Example 2-1, with the exception mat 5,7-dichloroanthranilic acid and ethylamine were used, was performed to give the title compound as a bright brown solid (64%).

¹H NMR (200 MHz, CD₃OD) δ 1.25 (t, 3H, J=7.3 Hz, CH₃), 3.15 (q, 2H, J=7.3 Hz, NHCH₂), 7.01 (d, 1H, J=2.0 Hz, ArH), 8.07 (d, 1H, J=2.0 Hz, ArH);

MS (EI) m/e 232 [M⁺], 205, 162, 72.

### Preparation Example 2-3

### Preparation of 2-amino-4,6-dichloro-N-propyl-benzamide (Intermediate I-3)

The same procedure as in Preparation Example 2-1, with the exception that 5,7-dichloroanthranilic acid and propylamine were used, was performed to give the title compound as a bright brown solid (76%).

m.p. 113-119° C.;

¹H NMR (200 MHz, CDCl₃) δ0.96 (t, 3H, J=7.3 Hz, CH₃), 1.65 (m, 2H, CH₂), 3.37 (q, 2H, J=6.5 Hz, NHCH₂), 6.07 (br s, 1H, NH), 6.58 (d, 1H, J=2.0 Hz, ArH), 6.72 (d, 1H, J=2.0 Hz, ArH);

MS (EI) m/e 246 [M⁺], 204, 144, 133.

### Preparation Example 2-4

### Preparation of 2-amino-N-butyl-4,6-dichloro-benzamide (Intermediate I-4)

The same procedure as in Preparation Example 2-1 with the exception that 5,7-dichloroanthranilic acid and butylamine were used, was performed to give the title compound as a brown solid (73%).

m.p. 92-98° C.;

¹H NMR (200 MHz, CDCl₃) δ 0.92 (t, 3H, J=7.3 Hz, CH₃), 1.42 (m, 2H, CH₂), 1.60 (m, 2H, CH₂), 3.40 (q, 2H, J=6.9 Hz, NHCH₂), 6.04 (br s, 1H, NH), 6.58 (d, 1H, J=2.0 Hz, ArH), 6.72 (d, 1H, J=2.0 Hz, ArH);

MS (EI) m/e 260 [M⁺], 246, 204, 188.

### Preparation Example 2-5

### Preparation of 2-amino-4,6-dichloro-N-(3-methyl-butyl)-benzamide (Intermediate I-5)

The same procedure as in Preparation Example 2-1 with the exception that 5,7-dichloroanthranilic acid and 3-methyl-butyl amine was performed to give the title compound as a bright brown solid (95%).

m.p. 148-150° C.;

¹H NMR (200 MHz, CDCl₃) δ 0.83 (d, 6H, J=7.0 Hz, CH₃×2), 1.41 (q, 2H, J=7.3 Hz, CH₂), 1.63 (m, 1H, CH), 3.41 (q, 2H, J=7.3 Hz, NHCH₂), 6.03 (br s, 1H, NH), 6.57 (d, 1H, J= 2.0 Hz, ArH), 6.71 (d, 1H, J=2.0 Hz, ArH);

MS (EI) m/e 274 [M⁺], 204, 188, 133, 73.

### Preparation Example 2-6

### Preparation of 2-amino-4,6-dichloro-N-cyclohexylmethyl-benzamide (Intermediate I-6)

The same procedure as in Preparation Example 2-1 with the exception that 5,7-dichloroanthranilic acid and cyclohexylmethylamine were used, was performed to give the title compound as a bright brown solid (91%).

m.p. 168-171° C.;

¹H NMR (200 MHz, CDCl₃) δ 0.81-0.99 (m, 5H, CH₂×2&CH), 1.12-1.20 (m, 6H, CH₂×3), 5.46 (s, 2H, NHCH₂), 6.68 (d, 1H, J=2.0 Hz, ArH), 6.96 (d, 1H, J=2.0 Hz, ArH);

MS (EI) m/e 299[M⁺].

### Preparation Example 2-7

### Preparation of 2-amino-4,6-dichloro-N-octyl-benzamide (Intermediate I-7)

The same procedure as in Preparation Example 2-1 with the exception that 5,7-dichloroanthranilic acid and 3-octylamine were used, was performed to give the title compound as a bright brown solid (97%).

m.p. 105-109° C.;

¹H NMR (200 MHz, CDCl₃+DMSO) δ 0.58 (m, 3H, CH₃), 0.94-0.96 (m, 10H, CH₂×5), 1.30 (m, 2H, CH₂), 3.05 (m, 2H, NHCH₂), 4.73 (br s, 2H, NH₂), 6.35 (d, 1H, J=1.2 Hz, ArH), 6.39 (d, 1H, J=1.2 Hz, ArH), 6.85 (br s, 1H, NH);

MS (EI) m/e 316 [M⁺], 246, 205.

### Preparation Example 2-8

### Preparation of 2-amino-4,6-dichloro-N-phenyl-benzamide (Intermediate I-8)

The same procedure as in Preparation Example 2-1 with the exception that 5,7-dichloroanthranilic acid and aniline were used, was performed to give the title compound as a brown solid (88%).

¹H NMR (200 MHz, CDCl₃) δ 7.18 (d, 1H, J=2.4 Hz, ArH), 7.32-7.62 (m, 6H, ArH), 7.72 (br s, 1H, NH);

MS (EI) m/e 280[M⁺].

### Preparation Example 2-9

### Preparation of 2-amino-4,6-dichloro-N-(4-methoxy-phenyl)-benzamide (Intermediate I-9)

The same procedure as in Preparation Example 2-1 with the exception that 5,7-dichloroanthranilic acid and 4-methoxy-phenyl amine were used, was performed to give the title compound as a white solid (95%).

¹H NMR (200 MHz, CDCl₃) δ 3.80 (s, 3H, OCH₃), 6.61 (d, 2H, J=7.8 Hz, ArH), 6.82 (br s, 1H, NH), 6.93 (d, 1H, J=2.0 Hz, ArH), 7.23 (d, 2H, J=7.8 Hz, ArH), 7.90 (d, 1H, J=2.0 Hz, ArH);

MS (EI) m/e 310 [M⁺].

### Preparation Example 2-10

### Preparation of 2-amino-N-benzyl-4,6-dichloro-benzamide (Intermediate I-10)

The same procedure as in Preparation Example 2-1 with the exception that 5,7-dichloroanthranilic acid and benzyl amine were used, was performed to give the title compound as a bright yellow solid (94%).

m.p. 90-96° C.;

¹H NMR (200 MHz, CDCl₃+CD₃OD) δ 4.27 (s, 2H, NHCH₂Ar), 6.93 (d, 1H, J=1.63 Hz, ArH), 7.22-7.35 (m, 5H, ArH), 8.10 (d, 1H, J=2.0 Hz, ArH);

MS (EI) m/e 294[M⁺], 106.

### Preparation Example 2-11

### Preparation of 2-amino-4,6-dichloro-N-(2-fluoro-benzyl)-benzamide (Intermediate I-11)

The same procedure as in Preparation Example 2-1 with, the exception that 5,7-dichloroanthranilic acid and 2-fluorobenzylamine were used, was performed to give the title compound as a brown solid (92%).

m.p. 154-158° C.;

¹H NMR (200 MHz, CDCl₃) δ 4.66 (d, 2H, J=6.2 Hz, NHCH₂Ar), 6.45 (br s, 1H, NH), 6.56 (d, 1H, J=2.4 Hz, ArH), 6.69 (d, 1H, J=2.4 Hz, ArH), 6.79-7.46 (m, 4H, ArH);

### Preparation Example 2-12

### Preparation of 2-amino-4,6-dichloro-N-(3-fluoro-benzyl)-benzamide (Intermediate I-12)

The same procedure as in Preparation Example 2-1 with the exception that 5,7-dichloroanthranilic acid and 3-fluorobenzylamine were used, was performed to give the title compound as a brown solid (92%).

m.p. 79-81° C.;

¹H NMR (200 MHz, CDCl₃) δ 4.61 (d, 2H, J=5.8 Hz, NHCH₂Ar), 4.80 (br s, 2H, NH₂), 6.46 (br s, 1H, NH), 6.58 (d, 1H, J=2.0 Hz, ArH), 6.74-7.37 (m, 4H, ArH);

MS (EI) m/e 312[M⁺].

### Preparation Example 2-13

### Preparation of 2-amino-4,6-dichloro-N-(4-fluoro-benzyl)-benzamide (Intermediate I-13)

The same procedure as in Preparation Example 2-1 with the exception that 5,7-dichloroanthranilic acid and 4-fluorobenzylamine were used, was performed to give the title compound as a brown solid (91%).

m.p. 89-94° C.;

¹H NMR (200 MHz, CDCl₃) δ 4.57 (d, 2H, J=5.6 Hz, NHCH₂Ar), 4.76 (br s, 2H, NH₂), 6.40 (br s, 1H, NH), 6.57 (d, 1H, J=2.0 Hz, ArH), 6.70 (d, 1H, J=1.6 Hz, ArH), 6.78-7.36 (m, 4H, ArH);

MS (EI) m/e 312 [M⁺].

### Preparation Example 2-14

### Preparation of 2-amino-N-(2-chloro-benzyl)-4,6-dichloro-benzamide (Intermediate I-14)

The same procedure as in Preparation Example 2-1 with the exception that 5,7-dichloroanthranilic acid and 2-chlorobenzylamine were used, was performed to give the title compound as a bright yellow solid (88%).

m.p. 160-164° C.;

¹H NMR (200 MHz, CDCl₃+CD₃OD) δ 4.70 (s, 2H, NHCH₂Ar), 6.71 (d, 2H, J=9.8 Hz, ArH), 7.06 (d, 1H, J=8.6 Hz, ArH), 7.22-7.51 (m, 3H, ArH), 8.16 (br s, 1H, NH);

MS (EI) m/e 319 [M⁺].

### Preparation Example 2-15

### 2-amino-4,6-dichloro-N-(3-chloro-benzyl)benzamide (Intermediate I-15)

The same procedure as in Preparation Example 2-1 with the exception that 5,7-dichloroanthranilic acid and 3-chlorobenzylamine were used, was performed to give the title compound as a bright yellow solid (97%).

m.p. 167-171° C.;

¹H NMR (200 MHz, CDCl₃+CD₃OD) δ 4.57 (s, 2H, NHCH₂Ar), 6.71 (d, 1H, J=1.2 Hz, ArH), 6.98 (d, 1H, J=1.2 Hz, ArH), 7.15-7.38 (m, 4H, ArH), 8.12 (br s, 1H, NH);

MS (EI) m/e 329[M⁺].

### Preparation Example 2-16

### 2-amino-4,6-dichloro-N-(4-chloro-benzyl)benzamide (Intermediate I-16)

The same procedure as in Preparation Example 2-1 with the exception that 5,7-dichloroanthranilic acid and 4-chlorobenzylamine were used, was performed to give the title compound as a bright yellow solid (98%).

m.p. 174-178° C.;

¹H NMR (200 MHz, CDCl₃) δ 4.56 (d, 2H, J=6.0 Hz, NHCH₂Ar), 6.58 (d, 1H, J=1.8 Hz, ArH), 6.71 (d, 1H, J=1.8 Hz, ArH), 6.87-7.34 (m, 4H, ArH);

MS (EI) m/e 328 [M⁺].

### Preparation Example 2-17

### 2-amino-N-(2-bromo-benzyl)-4,6-dichloro-benzamide (Intermediate I-17)

The same procedure as in Preparation Example 2-1 with the exception that 5,7-dichloroanthranilic acid and 2-bromobenzylamine were used, was performed to give the title compound as a bright yellow solid (90%).

m.p. 163-167° C.;

¹H NMR (200 MHz, CD₃OD) δ 4.25 (s, 2H, NHCH₂Ar), 6.78 (d, 1H, J=1.6 Hz, ArH), 7.01-7.17 (m, 4H, ArH), 8.15 (d, 1H, J=1.6 Hz, ArH), 8.75 (br s, 1H, NH);

MS (EI) m/e 374[M⁺].

### Preparation Example 2-18

### Preparation of 2-amino-N-(3-bromo-benzyl)-4,6-dichloro-benzamide (Intermediate I-18)

The same procedure as in Preparation Example 2-1 with the exception that 5,7-dichloroanthranilic acid and 3-bromobenzylamine were used, was performed to give the title compound as a bright yellow solid (94%).

m.p. 157-159° C.;

¹H NMR (200 MHz, CDCl₃) δ 4.33 (s, 2H, NHCH₂Ar), 6.93 (d, 1H, J=1.6 Hz, ArH), 7.08-7.46 (m, 4H, ArH), 7.27 (d, 1H, J=1.6 Hz, ArH);

MS (EI) m/e 187 [M⁺].

### Preparation Example 2-19

### Preparation of 2-amino-N-(4-bromo-benzyl)-4,6-dichloro-benzamide(Intermediate I-19)

The same procedure as in Preparation Example 2-1 with the exception that 5,7-dichloroanthranilic acid and 4-bromobenzylamine were used, was performed to give the title compound as a bright yellow solid (94%).

m.p. 161-165° C.;

¹H NMR (200 MHz, CDCl₃) δ 4.23 (s, 2H, NHCH₂Ar), 6.90 (d, 1H, J=1.6 Hz, ArH), 7.14 (d, 2H, J=8.6 Hz, ArH), 7.33 (d, 2H, J=8.6 Hz, ArH), 8.22 (d, 1H, J=1.6 Hz, ArH), 8.92 (br s, 1H, NH);

MS (EI) m/e 187 [M⁺].

### Preparation Example 2-20

### Preparation of 2-amino-4,6-dichloro-N-(3-iodo-benzyl)-benzamide(Intermediate I-20)

The same procedure as in Preparation Example 2-1 with the exception that 5,7-dichloroanthranilic acid and 3-iodobenzylamine were used, was performed to give the title compound as a brown solid (93%).

m.p. 165-169° C.;

¹H NMR (200 MHz, CDCl₃) δ 4.57 (d, 2H, J=6.0 Hz, NHCH₂Ar), 4.77 (br s, 2H, NH₂), 6.64 (br s, 1H, NH), 6.59 (d, 1H, J=2.0 Hz, ArH), 6.71 (d, 1H, J=2.0 Hz, ArH), 6.85-7.96 (m, 4H, ArH);

MS (EI) m/e 448 [M⁺].

### Preparation Example 2-21

### Preparation of 2-amino-4,6-dichloro-N-(4-iodo-benzyl)-benzamide(Intermediate I-21)

The same procedure as in Preparation Example 2-1 with the exception that 5,7-dichloroanthranilic acid and 4-iodobenzylamine were used, was performed to give the title compound as a brown solid (88%).

m.p. 99-103° C.;

¹H NMR (200 MHz, CDCl₃) δ 4.56 (d, 2H, J=6.0 Hz, NHCH₂Ar), 6.38 (br s, 1H, NH), 6.58 (m, 1H, ArH), 6.71 (m, 1H, ArH), 7.09 (d, 2H, J=8.2 Hz, ArH), 7.65 (d, 2H, J=8.2 Hz, ArH);

MS (EI) m/e 421 [M⁺].

### Preparation Example 2-22

### Preparation of 2-amino-4,6-dichloro-N-(2-methyl-benzyl)-benzamide(Intermediate I-22)

The same procedure as in Preparation Example 2-1 with the exception that 5,7-dichloroanthranilic acid and 2-methylbenzylamine were used, was performed to give the title compound as a bright yellow solid (90%).

m.p. 174-178° C.;

¹H NMR (200 MHz, CD₃OD) δ 4.65 (d, 2H, J=5.8 Hz, NHCH₂Ar), 6.60 (d, 1H, J=1.6 Hz, ArH), 6.71 (d, 1H, J=1.6 Hz, ArH), 7.11-7.30 (m, 4H, ArH);

MS (EI) m/e 308 [M⁺].

### Preparation Example 2-23

### Preparation of 2-amino-4,6-dichloro-N-(3-methyl-benzyl)-benzamide(Intermediate I-25)

The same procedure as in Preparation Example 2-1 with the exception that 5,7-dichloroanthranilic acid and 3-methylbenzylamine were used, was performed to give the title compound as a bright yellow solid (91%).

m.p. 130-132° C.;

¹H NMR (200 MHz, CDCl₃+CD₃OD) δ 2.32 (s, 3H, CH₃), 4.32 (s, 2H, NHCH₂Ar), 6.64 (d, 1H, J=1.6 Hz, ArH), 6.71 (d, 1H, J=1.6 Hz, ArH), 7.01-7.24 (m, 4H, ArH);

MS (EI) m/e 308 [M⁺].

### Preparation Example 2-24

### Preparation of 2-amino-4,6-dichloro-N-(4-methyl-benzyl)-benzamide(Intermediate I-24)

The same procedure as in Preparation Example 2-1 with the exception that 5,7-dichloroanthranilic acid and 4-methylbenzylamine were used, was performed to give the title compound as a bright yellow solid (91%).

m.p. 136-145° C.;

¹H NMR (200 MHz, CD₃OD) δ 2.32 (s, 3H, CH₃), 4.32 (s, 2H, NHCH₂Ar), 7.01-7.37 (m, 6H, ArH);

MS (EI) m/e 309 [M⁺].

### Preparation Example 2-25

### Preparation of 2-amino-4,6-dichloro-N-(2-methoxy-benzyl)-benzamide(Intermediate I-25)

The same procedure as in Preparation Example 2-1 with the exception that 5,7-dichloroanthranilic acid and 2-methoxybenzylamine were used, was performed to give the title compound as a bright yellow solid (94%).

m.p. 98-101° C.;

¹H NMR (200 MHz, CD₃OD) δ 3.86 (s, 3H, OCH₃), 4.60 (s, 2H, NHCH₂Ar), 6.63-7.37 (m, 6H, ArH);

MS (EI) m/e 324[M⁺].

### Preparation Example 2-26

### Preparation of 2-amino-4,6-dichloro-N-(3-methoxy-benzyl)-benzamide(Intermediate I-26)

The same procedure as in Preparation Example 2-1 with the exception that 5,7-dichloroanthranilic acid and 3-methoxybenzylamine were used, was performed to give the title compound as a bright yellow solid (95%).

m.p. 120-125° C.;

¹H NMR (200 MHz, CDCl₃) δ 3.77 (s, 3H, OCH₃), 4.58 (d, 2H, J=5.8 Hz, NHCH₂Ar), 6.55 (br s, 1H, NH), 6.60 (d, 1H, J=1.8 Hz, ArH), 6.77 (d, 1H, J=1.8 Hz, ArH), 6.88-7.25 (m, 4H, ArH);

MS (EI) m/e 324 [M⁺].

### Preparation Example 2-27

### Preparation of 2-amino-4,6-dichloro-N-(4-methoxy-benzyl)-benzamide(Intermediate I-27)

The same procedure as in Preparation Example 2-1 with the exception that 5,7-dichloroanthranilic acid and 4-methoxybenzylamine were used, was performed to give the title compound as a bright yellow solid (88%).

m.p. 173-177° C.;

¹H NMR (200 MHz, CD₃OD) δ 3.76 (s, 3H, OCH₃), 4.87 (d, 2H, J=4.0 Hz, NHCH₂Ar), 6.84-7.37 (m, 6H, ArH);

MS (EI) m/e 325 [M⁺], 205.

### Preparation Example 2-28

### Preparation of 2-amino-4,6-dichloro-N-(2-nitro-benzyl)-benzamide(Intermediate I-28)

The same procedure as in Preparation Example 2-1 with the exception that 5,7-dichloroanthranilic acid and 2-nitrobenzylamine were used, was performed to give the title compound as a brown solid (96%).

m.p. 134-138° C.;

¹H NMR (200 MHz, CDCl₃) δ 4.88 (d, 2H, J=6.4 Hz, NHCH₂Ar), 6.57 (d, 1H, J=2.0 Hz, ArH), 6.68-6.70 (m, 1H, ArH), 6.94 (br s, 1H, NH), 7.29-8.19 (m, 4H, ArH);

MS (ET) m/e 338 [M⁺].

### Preparation Example 2-29

### Preparation of 2-amino-4,6-dichloro-N-(3-nitro-benzyl)-benzamide(Intermediate I-29)

The same procedure as in Preparation Example 2-1 with the exception that 5,7-dichloroanthranilic acid and 3-nitrobenzylamine were used, was performed to give the title compound as a brown solid (99%).

m.p. 170-172° C.;

¹H NMR (200 MHz, CDCl₃+DMSO) δ 4.67 (s, 2H, NHCH₂Ar), 6.66-6.71 (m, 1H, ArH), 6.80-6.81 (m, 1H, ArH), 7.32-8.60 (m, 4H, ArH);

MS (EI) m/e 339 [M⁺].

### Preparation Example 2-30

### Preparation of 2-amino-4,6-dichloro-N-(4-nitro-benzyl)-benzamide(Intermediate I-30)

The same procedure as in Preparation Example 2-1 with the exception that 5,7-dichloroanthranilic acid and 4-nitrobenzylamine were used, was performed to give the title compound as a bright yellow solid (89%).

m.p. 78-83° C.;

¹H NMR (200 MHz, CDCl₃+DMSO) δ 4.69 (s, 2H, NHCH₂Ar), 6.68 (s, 1H, ArH), 7.04 (s, 1H, ArH), 7.45-7.73 (m, 2H, ArH), 8.15-8.30 (m, 2H, ArH);

MS (EI) m/e 339 [M⁺].

### Preparation Example 2-31

### Preparation of 2-amino-4,6-dichloro-N-(4-cyano-benzyl)-benzamide (Intermediate I-31)

The same procedure as in Preparation Example 2-1 with the exception that 5,7-dichloroanthranilic acid and 4-cyanobenzylamine were used, was performed to give the title compound as a yellow solid (98%).

¹H NMR (200 MHz, CDCl₃) δ 4.03 (br s, 1H, NHCH₂Ar), 4.18 (br s, 2H, NH₂), 4.67 (d, 2H, J=6.2 Hz, NHCH₂Ar), 6.59 (d, 1H, J=1.6 Hz, ArH), 6.78 (d, 1H, J=1.6 Hz, ArH), 7.14 (d, 1H, J=8.2 Hz, ArH), 7.62 (d, 1H, J=8.2 Hz, ArH);

MS (EI) m/e 391 [M⁺].

### Preparation Example 2-32

### Preparation of 4-[(2-amino-4,6-dichloro-benzoylamino)-methyl]-benzoic acid methyl ester (Intermediate I-32)

The same procedure as in Preparation Example 2-1 with the exception that 5,7-dichloroanthranilic acid and 4-methoxycarbonylbenzylamine were used, was performed to give the title compound as a brown solid (48%).

m.p. 132-137° C.;

¹H NMR (200 MHz, CDCl₃) δ 3.89 (m, 3H, OMe), 4.72 (s, 2H, NHCH₂Ar), 6.63-6.71 (m, 1H, ArH), 6.88-6.89 (m, 1H, ArH), 7.27-8.24 (m, 4H, ArH);

MS (EI) m/e 348[M⁺].

### Preparation Example 2-33

### Preparation of 2-amino-4,6-dichloro-N-(3,4-dimethyl-benzyl)-benzamide (Intermediate I-33)

The same procedure as in Preparation Example 2-1 with the exception that 5,7-dichloroanthranilic acid and 3,4-dimethylbenzylamine were used, was performed to give the title compound as a brown solid (96%).

¹H NMR (200 MHz, CDCl₃) δ 1.97-2.25 (m, 6H, ArCH₃×2), 3.98 (s, 2H, NHCH₂Ar), 6.70-7.67 (m, 5H, ArH);

MS (EI) m/e 322 (M.

### Preparation Example 2-34

### Preparation 2-amino-4,6-dichloro-N-[(R)-1-phenyl-ethyl]-benzamide (Intermediate I-34)

The same procedure as in Preparation Example 2-1 with the exception that 5,7-dichloroanthranilic acid and (R)-1-phenylethylamine were used, was performed to give the title compound as a bright yellow solid (95%).

m.p. 115-119° C.;

¹H NMR (200 MHz, CDCl₃) δ 1.22 (d, 3H, J=7.0 Hz, CH₃), 4.90 (m, 1H, NHCH),

6.92 (d, 1H, J=2.0 Hz, ArH), 7.15 (m, 3H, ArH), 7.25 (m, 3H, ArH);

MS (EI) m/e 308 [M⁺].

### Preparation Example 2-35

### Preparation 2-amino-4,6-dichloro-N-[(S)-1-phenyl-ethyl]-benzamide(Intermediate I-35)

The same procedure as in Preparation Example 2-1 with the exception that 5,7-dichloroanthranilic acid and (S)-1-phenylethylamine were used, was performed to give the title compound as a bright yellow solid (92%).

m.p. 108-114° C.;

¹H NMR (200 MHz, CDCl₃) δ 1.23 (d, 3H, J=7.0 Hz, CH₃), 4.92 (m, 1H, NHCH), 6.95 (d, 1H, J=2.0 Hz, ArH), 7.13 (m, 3H, ArH), 7.25 (m, 3H, ArH);

MS (EI) m/e 308 [M⁺].

### Preparation Example 2-36

### Preparation of 2-amino-4,6-dichloro-N-phenethyl-benzamide(Intermediate I-36)

The same procedure as in Preparation Example 2-1 with the exception that 5,7-dichloroanthranilic acid and phenethylamine were used, was performed to give the title compound as a bright yellow solid (65%).

¹H NMR (200 MHz, CDCl₃) δ 2.88-2.98 (m, 2H, CH₂Ar), 3.69-3.79 (m, 2H, NHCH₂), 6.06 (br s, 1H, NH), 6.56 (d, 1H, J=1.0 Hz, ArH), 6.68 (d, 1H, J=1.0 Hz, ArH), 7.22-7.36 (m, 5H, ArH);

MS (EI) m/e 308 [M⁺], 189.

### Preparation Example 2-37

### Preparation of 2-amino-4,6-dichloro-N-(3-phenyl-propyl)-benzamide(Intermediate I-37)

The same procedure as in Preparation Example 2-1 with the exception that 5,7-dichloroanthranilic acid and 3-phenylpropylamine were used, was performed to give the title compound as a pale yellow solid (70%).

m.p. 136-138° C.;

¹H NMR (200 MHz, CDCl₃) 81.95 (m, 2H, CH₂), 2.69 (t, 2H, J=13 Hz, CH₂Ph), 3.48 (m, 2H, NHCH₂), 4.75 (s, 2H, NH₂), 6.07 (br s, 1H, NH), 6.55-6.59 (m, 1H, ArH), 6.71-6.73 (m, 1H, ArH), 7.17-7.33 (m, 5H, ArH);

MS (EI) m/e 322 [M⁺], 188.

### Preparation Example 2-38

### Preparation of 2-amino-4,6-dichloro-N-[3-(3,5-dimethyl-phenyl)-propyl]-benzamide(Intermediate I-38)

The same procedure as in Preparation Example 2-1 with the exception that 5,7-dichloroanthranilic acid and 3-(3,5-dimethyl-phenyl)-propylamine were used, was performed to give the title compound as a yellow solid (90%).

m.p. 103-108° C.;

¹H NMR (200 MHz, CDCl₃) δ 1.94 (m, 2H, CH₂), 2.25 (s, 6H, 2×CH₃), 2.60 (t, 2H, J=7.3 Hz, CH₂Ar), 3.42 (q, 2H, J=6.9 Hz, NHCH₂), 4.41 (br s, 2H, NH₂), 6.08 (br s, 1H, NH), 6.57 (d, 1H, J=2.0 Hz, ArH), 6.71 (d, 1H, J=2.0 Hz, ArH), 6.80-6.82 (m, 3H, ArH);

MS (EI) m/e 350 [M⁺], 204, 188, 146.

### Preparation Example 2-39

### Preparation of 2-amino-4,6-dichloro-N-[3-(3-isobutyl-phenyl)-propyl]-benzamide(Intermediate I-39)

The same procedure as in Preparation Example 2-1 with the exception that 5,7-dichloroanthranilic acid and 3-(3-isobutyl-phenyl)-propylamine were used, was performed to give the title compound as a yellow syrup (90%).

¹H NMR (200 MHz, CDCl₃) δ 0.95 (d, 6H, J=7.0 Hz, 2×CH₃), 1.66 (m, 2H, CH₂), 1.82-2.04 (m, 3H, ArCH₂&CH), 2.63 (m, 2H, CH₂Ar), 3.29 (m, 2H, NHCH₂), 4.76 (br s, 2H, NH₂), 5.93 (br s, 1H, NH), 6.56 (s, 1H, ArH), 6.70 (s, 1H, ArH), 7.14-7.33 (m, 4H, ArH);

MS (EI) m/e 378 [M⁺], 204, 188, 131.

### Preparation Example 2-40

### Preparation of 2-amino-4,6-dichloro-N-naphthalen-1-ylmethyl-benzamide(Intermediate I-40)

The same procedure as in Preparation Example 2-1 with the exception that 5,7-dichloroanthranilic acid and naphthalen-1-ylmethylamine were used, was performed to give the title compound as a yellow solid (62%).

m.p. 160-164° C.;

¹H NMR (200 MHz, CDCl₃) δ 4.77 (s, 2H, NH₂), 5.08 (d, 2H, J=3.5 Hz, NHCH₂Ar), 6.31 (s, 1H, NH), 6.55 (d, 1H, J=1.8 Hz, ArH), 6.66 (d, 1H, J=1.8 Hz, ArH), 7.41-7.56 (m, 4H, ArH), 7.87 (d, 1H, J=8.1 Hz, ArH), 7.87 (d, 1H, J=7.2 Hz, ArH), 8.07 (d, 1H, J=7.5 Hz, ArH);

MS (EI) m/e 344 [M⁺], 156, 141.

### Preparation Example 2-41

### Preparation of 2-amino-4,6-dichloro-N-(5-methyl-furan-2-ylmethyl)-benzamide(Intermediate I-41)

The same procedure as in Preparation Example 2-1 with the exception that 5,7-dichloroanthranilic acid and 5-methyl-furan-2-ylmethylamine were used, was performed to give the title compound as a yellow solid (97%).

m.p. 109-111° C.;

¹H NMR (200 MHz, CDCl₃+DMSO) δ 2.25 (m, 3H, CH₃), 4.46 (m, 2H, NHCH₂), 5.07 (br s, 2H, NH₂), 5.89-5.91 (m, 1H, ArH), 6.13-6.16 (m, 1H, ArH), 6.61-6.68 (m, 2H, ArH);

MS (EI) m/e 298 [M⁺], 227, 204, 110, 95.

### Preparation Example 2-42

### Preparation of 2-amino-4,6-dichloro-N-pyridin-4-ylmethyl-benzamide(Intermediate I-42)

The same procedure as in Preparation Example 2-1 with the exception that 5,7-dichloroanthranilic acid and pyridin-4-ylmethylamine were used, was performed to give the title compound as a brown solid (47%).

m.p. 163-169° C.;

¹H NMR (200 MHz, CDCl₃+DMSO-d₆) δ 4.45 (d, 2H, J=5.8 Hz, NHCH₂Ar), 5.57 (br s, 2H, NH₂), 6.64 (d, 1H, J=2.0 Hz, ArH), 6.71 (d, 1H, J=2.0 Hz, ArH), 7.34-7.37 (m, 2H, ArH), 8.49-8.52 (m, 2H, ArH), 9.01-9.07 (m, 1H, NH);

MS (EI) m/e 295 [M⁺],

### Preparation Example 3

### Preparation of Intermediate H (Method A)

### Preparation Example 3-1

### Preparation of 5,7-dichloro-3-methyl-1H-quinazoline-2,4-dione (Intermediate II-1)

Triphosgene (0.4 mmol) was allowed to react for 2 hours in a solution of Intermediate I-1 (1.0 mmol) in 1,4-dioxdane (20 ml) or tetrahydrofuran (20 ml) with fluxing. Following the dissolution of the starting material benzamide, the solvent was removed in a vacuum. The residue was washed with 1 N hydrochloric acid (100 ml), and recrystallization in methanol and ethylacetate (or purification by flash column chromatography (eluent: n-hexane and ethylacetate)) gave a yellow solid (99%).

m.p. 247-250° C.;

¹H NMR (200 MHz, CDCl₃+DMSO) δ 3.38 (s, 3H, NCH₃), 7.16 (m, 2H, ArH), 11.46 (s, 1H, NH);

MS (EI) m/e 246 [M⁺], 231, 199, 176, 160, 126.

### Preparation Example 3-2

### Preparation of 5,7-Dichloro-3-ethyl-1H-quinazoline-2,4-dione (Intermediate II-2)

The same procedure as in Preparation Example 3-1, with the exception that Intermediate 1-2 and triphosgene were used, was performed to give the title compound as a yellow solid (64%).

¹H NMR (200 MHz, CDCl₃+CD₃OD) δ 1.23 (t, 3H, J=7.3 Hz, CH₃), 4.00 (q, 2H, J=7.3 Hz, NCH₂), 7.08 (d, 1H, J=2.0 Hz, ArH), 7.21 (d, 1H, J=2.0 Hz, ArH);

MS (EI) m/e 259 [M⁺], 230, 187, 160.

### Preparation Example 3-3

### Preparation of 5,7-dichloro-3-propyl-1H-quinazoline-2,4-dione (Intermediate II-3)

The same procedure as in Preparation Example 3-1 with the exception that Intermediate I-3 and triphosgene were used, was performed to give the title compound as a pale yellow solid (96%).

m.p. 223-234° C.;

¹H NMR (200 MHz, DMSO) δ 0.96 (t, 3H, J=6.5 Hz, CH₃), 1.73 (m, 2H, CH₂), 3.97 (m, 2H, J=6.5 Hz, NCH₂), 7.02 (d, 1H, J=2.0 Hz, ArH), 7.24 (m, 1H, ArH), 9.84 (br s, 1H, NH);

MS (EI) m/e 272 [M⁺], 231, 160, 72.

### Preparation Example 3-4

### Preparation of 3-butyl-5,7-dichloro-1H-quinazoline-2,4-dione (Intermediate II-4)

The same procedure as in Preparation Example 3-1 with the exception that Intermediate 1-4 and triphosgene were used, was performed to give the title compound as a pale yellow solid (90%).

m.p. 198-201° C.;

¹H NMR (200 MHz, DMSO) d 0.94 (t, 3H, J=7.3 Hz, CH₃), 1.37 (m, 2H, CH₂), 1.67 (m, 2H, CH₂), 4.00 (t, 2H, J=7.3 Hz, NCH₂), 7.05 (d, 1H, J=2.0 Hz, ArH), 7.24 (d, 1H, J=2.0 Hz, ArH), 10.35 (s, 1H, NH);

MS (EI) m/e 286 [M⁺], 231, 214, 197, 160.

### Preparation Example 3-5

### Preparation of 5,7-dichloro-3-(3-methyl-butyl)-1H-quinazoline-2,4-dione (Intermediate II-5)

The same procedure as in Preparation Example 3-1 with the exception that Intermediate I-5 and triphosgene were used, was performed to give the title compound as a white solid (80%).

m.p. 227-229° C.;

¹H NMR (200 MHz, DMSO) δ 0.98 (d, 6H, J=6.5 Hz, CH₃×2), 1.56-1.74 (m, 3H, CH₂&CH), 4.02 (t, 2H, J=7.3 Hz, NCH₂), 7.04 (d, 1H, J=2.0 Hz, ArH), 7.24 (d, 1H, J=2.0 Hz, ArH), 10.65 (s, 1H, NH);

MS (EI) We 300 [M⁺], 231, 198, 160.

### Preparation Example 3-6

### Preparation of 5,7-dichloro-3-cyclohexylmethyl-1H-quinazoline-2,4-dione (Intermediate II-6)

The same procedure as in Preparation Example 3-1 with the exception that Intermediate I-6 and triphosgene were used, was performed to give the title compound as a white solid (52%).

m.p. 238-243° C.;

¹H NMR (200 MHz, DMSO-d₆) δ 0.93 (m, 5H, CH₂×2&CH), 1.57-1.62 (m, 6H, CH₂×3), 3.68 (d, 2H, J=7.4 Hz, NCH₂), 7.14 (d, 1H, J=2.2 Hz, ArH), 7.35 (d, 1H, <J=2.2 Hz, ArH), 11.61 (br s, 1H, NH);

MS (EI) m/e 328 [M⁺].

### Preparation Example 3-7

### Preparation of 5,7-dichloro-3-octyl-1H-quinazoline-2,4-dione (Intermediate II-7)

The same procedure as in Preparation Example 3-1 with the exception that Intermediate I-7 and triphosgene were used, was performed to give the title compound as a white solid (50%).

m.p. 161-163° C.;

¹H NMR (200 MHz, DMSO) d 0.84 (m, 3H, CH₃), 1.26 (m, 10H, CH₂×5), 1.54 (m, 2H, CH₂), 3.81 (m, 2H, NCH₂), 7.14 (d, 1H, J=1.6 Hz, ArH), 7.36 (d, 1H, J=1.6 Hz, ArH), 11.60 (br s, 1H, NH);

MS (EI) m/e 342 [M⁺], 231, 160, 126, 112, 72.

### Preparation Example 3-8

### Preparation of 5,7-dichloro-3-phenyl-1H-quinazoline-2,4-dione (Intermediate II-8)

The same procedure as in Preparation Example 3-1 with the exception that Intermediate I-8 and triphosgene were used, was performed to give the title compound as a white solid (57%).

m.p. 314-319° C.;

¹H NMR (200 MHz, DMSO-d₆) δ 7.20 (d, 1H, J=2.0 Hz, ArH), 7.28-7.51 (m, 6H, ArH), 11.76 (s, 1H, NH);

MS (EI) m/e 306 [M⁺].

### Preparation Example 3-9

### Preparation of 5,7-dichloro-3 (4-methoxy-phenyl)-1H-quinazoline-2,4-dione (Intermediate II-9)

The same procedure as in Preparation Example 3-1 with the exception that Intermediate 1-9 and triphosgene were used, was performed to give the title compound as a white solid (67%).

m.p. 294-296° C.;

¹H NMR (200 MHz, DMSO-d₆) δ 3.84 (s, 3H, OCH₃), 7.20 (d, 1H, J=2.0 Hz, ArH), 7.23 (d, 2H, J=7.8 Hz, ArH), 7.39 (d, 2H, J=7.8 Hz, ArH), 8.10 (d, 1H, J=2.0 Hz, ArH), 11.20 (br s, 1H, NH);

MS (EI) m/e 336 [M⁺].

### Preparation Example 3-10

### Preparation of 3-benzyl-5,7-dichloro-1H-quinazoline-2,4-dione (Intermediate II-10)

The same procedure as in Preparation Example 3-1 with the exception that Intermediate I-10 and triphosgene were used, was performed to give the title compound as a white solid (63%).

m.p. 260-265° C.;

¹H NMR (200 MHz, CDCl₃+CD₃OD) δ 5.18 (s, 2H, NCH₂Ar), 7.04 (d, 1H, J=2.0 Hz, ArH), 7.20 (d, 1H, J=2.0 Hz, ArH), 7.27-7.50 (m, 5H, ArH);

MS (EI) m/e 320 [M⁺], 91.

### Preparation Example 3-11

### Preparation of 5,7-dichloro-3-(2-fluoro-benzyl)-1H-quinazoline-2,4-dione (Intermediate II-11)

The same procedure as in Preparation Example 3-1 with the exception that Intermediate I-11 and triphosgene were used, was performed to give the title compound as a white solid (80%).

m.p. 275-278° C.;

¹H NMR (200 MHz, DMSO-d₆) δ 5.08 (s, 2H, NCH₂Ar), 7.07-7.41 (m, 6H, ArH), 11.80 (s, 1H, NH);

MS (EI) m/e 338[M⁺].

### Preparation Example 3-12

### Preparation of 5,7-dichloro-3-(3-fluoro-benzyl)-1H-quinazoline-2,4-dione (Intermediate II-12)

The same procedure as in Preparation Example 3-1 with the exception that Intermediate I-12 and triphosgene were used, was performed to give the title compound as a white solid (60%).

m.p. 267-270° C.;

¹H NMR (200 MHz, DMSO-d₆) δ 5.03 (s, 2H, NCH₂Ar), 7.06-7.40 (m, 6H, ArH), 11.76 (s, 1H, NH);

MS (Er) m/e 338[M⁺].

### Preparation Example 3-13

### Preparation of 5,7-dichloro-3-(4-fluoro-benzyl)-1H-quinazoline-2,4-dione (Intermediate II-13)

The same procedure as in Preparation Example 3-1 with the exception that Intermediate I-13 and triphosgene were used, was performed to give the title compound as a white solid (69%).

m.p. 301-304° C.;

¹H NMR (200 MHz, DMSO-d₆) δ 5.00 (s, 2H, NCH₂Ar), 7.08-7.18 (m, 4H, ArH), 7.35-7.42 (m, 2H, ArH), 11.74 (s, 1H, NH);

MS (EI) m/e 338 [M⁺].

### Preparation Example 3-14

### Preparation of 5,7-dichloro-3-(2-chloro-benzyl)-1H-quinazoline-2,4-dione (Intermediate II-14)

The same procedure as in Preparation Example 3-1 with the exception that Intermediate I-14 and triphosgene were used, was performed to give the title compound as a white solid (60%).

m.p. 193-198° C.;

¹H NMR (200 MHz, DMSO) δ 5.07 (s, 2H, NCH₂Ar), 7.14 (m, 1H, ArH), 7.22-7.51 (m, 5H, ArH);

MS (EI) m/e 350 [M⁺].

### Preparation Example 3-15

### Preparation of 5,7-dichloro-3-(3-chloro-benzyl)-1H-quinazoline-2,4-dione (Intermediate II-15)

The same procedure as in Preparation Example 3-1 with the exception that Intermediate I-15 and triphosgene were used, was performed to give the title compound as a white solid (93%).

m.p. 276-279° C.;

¹H NMR (200 MHz, CDCl₃+DMSO) δ 4.09 (s, 2H, NCH₂Ar), 6.27 (d, 1H, J=2.0 Hz, ArH), 6.35-6.40 (m, 4H, ArH), 6.46 (d, 1H, J=2.0 Hz, ArH), 10.84 (br s, 1H, NH);

MS (EI) m/e 355 [M⁺].

### Preparation Example 3-16

### Preparation of 5,7-dichloro-3-(4-chloro-benzyl)-1H-quinazoline-2,4-dione (Intermediate II-16)

The same procedure as in Preparation Example 3-1 with the exception that Intermediate I-16 and triphosgene were used, was performed to give the title compound as a white solid (89%).

m.p. 277-280° C.;

¹H NMR (200 MHz, DMSO) δ 5.01 (s, 2H, NCH₂Ar), 7.10 (d, 1H, J=2.2 Hz, ArH), 7.27-7.39 (m, 4H, ArH), 7.50 (d, 1H, J=2.2 Hz, ArH), 11.77 (s, 1H, NH);

MS (EI) m/e 354 [M⁺].

### Preparation Example 3-17

### Preparation of 3-(2-bromo-benzyl)-5,7-dichloro-1H-quinazoline-2,4-dione (Intermediate II-17)

The same procedure as in Preparation Example 3-1 with the exception that Intermediate I-17 and triphosgene were used, was performed to give the title compound as a white solid (56%).

m.p. 170-174° C.;

¹H NMR (200 MHz, DMSO) δ 5.02 (s, 2H, NCH₂Ar), 7.10 (d, 1H, J=> 2.2 Hz, ArH), 7.22-7.38 (m, 3H, ArH), 7.43 (m, 1H, ArH), 7.67 (m, 1H, ArH);

MS (EI) m/e 490[M⁺].

### Preparation Example 3-18

### Preparation of 3-(3-bromo-benzyl)-5,7-dichloro-1H-quinazoline-2,4-dione (Intermediate II-18)

The same procedure as in Preparation Example 3-1 with the exception that Intermediate I-18 and triphosgene were used, was performed to give the title compound as a white solid (68%).

m.p. 161-165° C.;

¹H NMR (200 MHz, DMSO) δ 5.01 (s, 2H, NCH₂Ar), 7.18 (d, 1H, J=1.6 Hz, ArH), 7.24 (d, 1H, J=1.6 Hz, ArH), 7.27-7.54 (m, 4H, ArH), 11.76 (s, 1H, NH);

MS (EI) m/e 399 [M⁺], 230, 186.

### Preparation Example 3-19

### Preparation of 3-(4-bromo-benzyl)-5,7-dichloro-1H-quinazoline-2,4-dione (Intermediate II-19)

The same procedure as in Preparation Example 3-1 with the exception that Intermediate I-19 and triphosgene were used, was performed to give the title compound as a white solid (75%).

m.p. 181-184° C.;

¹H NMR (200 MHz, DMSO) δ 4.00 (s, 2H, NCH₂Ar), 7.19 (d, 1H, J=2.2 Hz, ArH), 7.31 (d, 2H, J=8.4 Hz, ArH), 7.40 (d, 1H, J=2.2 Hz, ArH), 7.53 (d, 2H, J=8.0 Hz, ArH);

MS (EI) m/e 400 [M⁺].

### Preparation Example 3-20

### Preparation of 5,7-dichloro-3-(3-iodo-benzyl)-1H-quinazoline-2,4-dione (Intermediate II-20)

The same procedure as in Preparation Example 3-1 with the exception that Intermediate I-20 and triphosgene were used, was performed to give the title compound as a white solid (64%).

m.p. 299-302° C.;

¹H NMR (200 MHz, DMSO-d₆) δ 4.98 (s, 2H, NCH₂Ar), 7.07 (dd, 1H, J=7.2, 7.4 Hz, ArH), 7.17 (d, 1H, J=2.2 Hz, ArH), 7.31 (dd, 1H, J=7.4, 2.8 Hz, ArH), 7.38 (d, 1H, J=2.0 Hz, ArH), 7.38 (d, 1H, J=2.0 Hz, ArH), 7.60 (dd, 1H, J=1.2, 2.8 Hz, ArH), 11.75 (s, 1H, NH);

MS (EI) m/e 445[M⁺].

### Preparation Example 3-21

### Preparation of 5,7-dichloro-3-(4-iodo-benzyl)-1H-quinazoline-2,4-dione (Intermediate II-21)

The same procedure as in Preparation Example 3-1 with the exception that Intermediate I-21 and triphosgene were used, was performed to give the title compound as a white solid (59%).

m.p. 314-316° C.;

¹H NMR (200 MHz, DMSO-d₆) δ 4.97 (s, 2H, NCH₂Ar), 7.11 (d, 2H, J=8.2 Hz, ArH), 7.17 (d, 1H, J=2.0 Hz, ArH), 7.38 (d, 1H, J=2.0 Hz, ArH), 7.64 (d, 2H, J=8.2 Hz, ArH), 11.75 (s, 1H, NH);

MS (EI) m/e 445 [M⁺].

### Preparation Example 3-22

### Preparation of 5,7-dichloro-3-(2-methyl-benzyl)-1H-quinazoline-2,4-dione (Intermediate II-22)

The same procedure as in Preparation Example 3-1 with the exception that Intermediate I-22 and triphosgene were used, was performed to give the title compound as a white solid (68%).

m.p. 274-276° C.;

¹H NMR (200 MHz, DMSO) δ 2.50 (s, 3H, CH₃), 5.00 (s, 2H, NCH₂Ar), 6.95 (d, 1H, J=6.8 Hz, ArH), 7.03-7.41 (m, 5H, ArH);

MS (EI) m/e 334 [M⁺].

### Preparation Example 3-23

### Preparation of 5,7-dichloro-3-(3-methyl-benzyl)-1H-quinazoline-2,4-dione (Intermediate II-23)

The same procedure as in Preparation Example 3-1 with the exception that Intermediate I-23 and triphosgene were used, was performed to give the title compound as a white solid (91%).

m.p. 223-230° C.;

¹H NMR (200 MHz, DMSO) δ 2.26 (s, 3H, CH₃), 4.99 (s, 2H, NCH₂Ar), 7.07-7.15 (m, 4H, ArH), 7.19 (d, 1H, J=2.2 Hz, ArH), 7.39 (d, 2H, J=2.2 Hz, ArH);

MS (EI) m/e 354 [M⁺].

### Preparation Example 3-24

### Preparation of 5,7-dichloro-3-(4-methyl-benzyl)-1H-quinazoline-2,4-dione (Intermediate II-24)

The same procedure as in Preparation Example 3-1 with the exception that Intermediate I-24 and triphosgene were used, was performed to give the title compound as a white solid (65%).

m.p. 210-218° C.;

¹H NMR (200 MHz, DMSO) δ 2.27 (s, 3H, CH₃), 4.98 (s, 2H, NCH₂Ar), 7.08-7.19 (m, 2H, ArH), 7.13 (d, 1H, J=2.0 Hz, ArH), 7.26 (d, 1H, J=2.0 Hz, ArH), 7.55-7.60 (m, 2H, ArH);

MS (EI) m/e 354[M⁺].

### Preparation Example 3-25

### Preparation of 5,7-dichloro-3-(2-methoxy-benzyl)-1H-quinazoline-2,4-dione (Intermediate II-25)

The same procedure as in Preparation Example 3-1 with the exception that Intermediate I-25 and triphosgene were used, was performed to give the title compound as a white solid (61%).

m.p. 143-146° C.;

¹H NMR (200 MHz, DMSO) δ 3.85 (s, 3H, OCH₃), 5.00 (s, 2H, NCH₂Ar), 6.87 (m, 1H, ArH), 7.03 (d, 2H, J-8.6 Hz, ArH), 7.22 (m, 2H, ArH), 7.41 (m, 1H, ArH) 11.77 (s, 1H, NH);

MS (EI) m/e 350 [M⁺].

### Preparation Example 3-26

### Preparation of 5,7-dichloro-3-(3-methoxy-benzyl)-1H-quinazoline-2,4-dione (Intermediate II-26)

The same procedure as in Preparation Example 3-1 with the exception that Intermediate I-26 and triphosgene were used, was performed to give the title compound as a bright yellow solid (70%).

m.p. 216-220° C.;

¹H NMR (200 MHz, CDCl₃) δ 3.86 (s, 3H, OCH₃), 5.23 (s, 2H, NCH₂Ar), 6.79 (d, 1H, J=1.8 Hz, ArH), 6.83 (d, 1H, J=1.8 Hz, ArH), 6.99-7.29 (m, 11H, ArH), 10.25 (br s, 1H, NH);

MS (EI) m/e 350 [M⁺].

### Preparation Example 3-27

### Preparation of 5,7-dichloro-3-(4-methoxy-benzyl)-1H-quinazoline-2,4-dione (Intermediate II-27)

The same procedure as in Preparation Example 3-1 with the exception that Intermediate I-27 and triphosgene were used, was performed to give the title compound as a white solid (76%).

m.p. 180-183° C.;

¹H NMR (200 MHz, CDCl₃+DMSO) δ 3.28 (s, 3H, OCH₃), 5.03 (s, 2H, NCH₂Ar), 6.79 (d, 1H, J=1.6 Hz, ArH), 6.83 (d, 1H, J=1.6 Hz, ArH), 7.34-7.38 (m, 4H, ArH);

MS (EI) m/e 350 [M⁺], 121.

### Preparation Example 3-28

### Preparation of 5,7-dichloro-3-(2-nitro-benzyl)-1H-quinazoline-2,4-dione (Intermediate II-28)

The same procedure as in Preparation Example 3-1 with the exception that Intermediate I-28 and triphosgene were used, was performed to give the title compound as a white solid (72%).

m.p. 307-313° C.

¹H NMR (200 MHz, DMSO-d₆) δ 5.30 (s, 2H, NCH₂Ar), 7.21 (d, 1H, J=2.0 Hz, ArH), 7.41 (d, 1H, J=2.0 Hz, ArH), 7.50-7.69 (m, 3H, ArH), 8.07 (d, 1H, J=7.6 Hz, ArH), 11.83 (s, 1H, NH);

MS (EI) m/e 365 [M⁺].

### Preparation Example 3-29

### Preparation of 5,7-dichloro-3-(3-nitro-benzyl)-1H-quinazoline-2,4-dione (Intermediate II-29)

The same procedure as in Preparation Example 3-1 with the exception that Intermediate I-29 and triphosgene were used, was performed to give the title compound as a white solid (62%).

m.p. 314-317° C.;

¹H NMR (200 MHz, DMSO-d₆) δ 5.14 (s, 2H, NCH₂Ar), 7.18 (d, 1H, J=2.0 Hz, ArH), 7.40 (d, 1H, J=2.0 Hz, ArH), 7.57 (t, 1H, J=7.8 Hz, ArH), 7.78 (d, 1H, J=7.8 Hz, ArH), 8.11 (d, 1H, J=7.8 Hz, ArH), 8.20 (s, 1H, ArH), 11.80 (s, 1H, NH);

MS (EI) m/e 365 [M⁺].

### Preparation Example 3-30

### Preparation of 5,7-dichloro-3-(4-nitro-benzyl)-1H-quinazoline-2,4-dione (Intermediate II-30)

The same procedure as in Preparation Example 3-1 with the exception that Intermediate I-30 and triphosgene were used, was performed to give the title compound as a white solid (76%).

m.p. 227-230° C.;

¹H NMR (200 MHz, DMSO) δ 5.14 (s, 2H, NCH₂Ar), 7.20 (s, 1H, ArH), 7.41 (s, 1H, ArH), 7.61 (d, 2H, J=8.6 Hz, ArH), 8.19 (d, 2H, J=8.6 Hz, ArH), 11.83 (s, 1H, NH);

MS (EI) m/e 365 [M⁺].

### Preparation Example 3-31

### Preparation of 4-(5,7-dichloro-2,4-dioxo-1,4-dihydro-2H-quinazoline-3-ylmethyl)-benzonitrile (Intermediate H-31)

The same procedure as in Preparation Example 3-1 with the exception that Intermediate I-31 and triphosgene were used, was performed to give the title compound as a white solid (70%).

m.p. 317-320° C.;

¹H NMR (200 MHz, DMSO-d₆) δ 5.09 (s, 2H, NCH₂Ar), 7.18 (d, 1H, J=2.0 Hz, ArH), 7.39 (d, 1H, J=2.0 Hz, ArH), 7.48 (d, 2H, J=8.4 Hz, ArH), 7.76 (d, 2H, J=8.4 Hz, ArH), 11.80 (br s, 1H, NH);

MS (EI) m/e 345[M⁺].

### Preparation Example 3-32

### Preparation of 4-(5,7-dichloro-2,4-dioxo-1,4-dihydro-2H-quinazoline-3-ylmethyl)-benzoic acid methyl ester(Intermediate II-32)

The same procedure as in Preparation Example 3-1 with the exception that Intermediate I-32 and triphosgene were used, was performed to give the title compound as a white solid (59%).

m.p. 301-307° C.;

¹H NMR (200 MHz, DMSO-d₆) δ 3.83 (s, 3H, OMe), 5.09 (s, 2H, NCH₂Ar), 7.19 (d, 1H, J=2.0 Hz, ArH), 7.39 (d, 1H, J-2.0 Hz, ArH), 7.42 (d, 2H, J=8.0 Hz, ArH), 7.88 (d, 2H, J=8.0 Hz, ArH), 11.77 (s, 1H, NH);

MS (EI) m/e 378 [M⁺].

### Preparation Example 3-33

### Preparation of 5,7-dichloro-3-(3,4-dimethyl-benzyl)-1H-quinazoline-2,4-dione (Intermediate II-33)

The same procedure as in Preparation Example 3-1 with the exception that Intermediate I-33 and triphosgene were used, was performed to give the title compound as a white solid (50%).

m.p. 272-275° C.;

¹H NMR (200 MHz, DMSO-d₆) δ 2.80-2.84 (m, 6H, ArCH₃×2), 5.28 (s, 2H, NCH₂Ar), 7.32-7.40 (m, 3H, ArH), 7.50 (d, 1H, J=2.0 Hz, ArH), 7.70 (d, 1H, J=2.0 Hz, ArH), 12.03 (br s, 1H, NH);

MS (EI) m/e 348[M⁺].

### Preparation Example 3-34

### Preparation of 5,7-dichloro-3-[(R)-1-phenyl-ethyl]-1H-quinazoline-2,4-dione (Intermediate II-34)

The same procedure as in Preparation Example 3-1 with the exception that Intermediate I-34 and triphosgene were used, was performed to give the title compound as a bright yellow solid (90%).

m.p. 205-210° C.;

¹H NMR (200 MHz, CDCl₃) δ 1.55 (d, 3H, J=7.0 Hz, CH₃), 4.98 (m, 1H, NCH), 7.05 (d, 1H, J=2.0 Hz, ArH), 7.35-7.37 (m, 5H, ArH), 8.35 (d, 1H, J=2.0 Hz, ArH);

MS (EI) m/e 334 [M⁺].

### Preparation Example 3-35

### Preparation of 5,7-dichloro-3-[(S)-1-phenyl-ethyl]-1H-quinazoline-2,4-dione (Intermediate II-35)

The same procedure as in Preparation Example 3-1 with the exception that Intermediate I-35 and triphosgene were used, was performed to give the title compound as a bright yellow solid (95%).

m.p. 203-210° C.;

¹H NMR (200 MHz, CDCl₃) δ 1.57 (d, 3H, J=7.0 Hz, CH₃), 4.96 (m, 1H, NCH), 7.06 (d, 1H, J=2.0 Hz, ArH), 7.35-7.38 (m, 5H, ArH), 8.35 (d, 1H, J=2.0 Hz, ArH);

MS (EI) m/e 334[M⁺].

### Preparation Example 3-36

### Preparation of 5,7-dichloro-3-phenethyl-1H-quinazoline-2,4-dione (Intermediate II-36)

The same procedure as in Preparation Example 3-1 with the exception that Intermediate I-36 and triphosgene were used, was performed to give the title compound as a white solid (81%).

m.p. 230-232° C.;

¹H NMR (200 MHz, DMSO) δ 2.95 (m, 2H, CH₂Ar), 4.20 (m, 2H, NCH₂), 7.02 (d, 1H, J=2.0 Hz, ArH), 7.22-7.32 (m, 5H, ArH);

MS (EI) m/e 334[M⁺], 104.

### Preparation Example 3-37

### Preparation of 5,7-dichloro-3-(3-phenyl-propyl)-1H-quinazoline-2,4-dione (Intermediate II-37)

The same procedure as in Preparation Example 3-1 with the exception that Intermediate I-37 and triphosgene were used, was performed to give the title compound as a white solid (85%).

m.p. 194-195° C.;

¹H NMR (200 MHz, DMSO) δ 2.09 (m, 2H, CH₂), 2.72 (t, 2H, J=7.3 Hz, CH₂Ar), 4.07 (t, 2H, J=13 Hz, NCH₂), 6.99-7.26 (m, 7H, ArH), 10.48 (s, 1H, NH);

MS (EI) m/e 348 [M⁺], 244, 231, 118.

### Preparation Example 3-38

### Preparation of 5,7-dichloro-3-[3-(3,5-dimethyl-phenyl)-propyl]-1H-quinazoline-2,4-dione (Intermediate II-38)

The same procedure as in Preparation Example 3-1 with the exception that Intermediate I-38 and triphosgene were used, was performed to give the title compound as an ivory solid (92%).

m.p. 205-208° C.;

¹H NMR (200 MHz, DMSO) δ 2.06 (m, 2H, CH₂), 2.23 (s, 6H, 2×CH₃), 2.63 (t, 2H, J=7.3 Hz, CH₂Ar), 4.08 (t, 2H, J=6.5 Hz, NCH₂), 6.75 (s, 1H, ArH), 6.82 (s, 2H, ArH), 6.99 (d, 2H, J=2.0 Hz, ArH), 7.26 (m, 1H, ArH), 9.95 (s, 1H, NH);

MS (EI) m/e 376 [M⁺], 231, 198, 146.

### Preparation Example 3-39

### Preparation of 5,7-dichloro-3-[3-(3-isobutyl-phenyl)-propyl]-1H-quinazoline-2,4-dione (Intermediate II-39)

The same procedure as in Preparation Example 3-1 with the exception that Intermediate I-39 and triphosgene were used, was performed to give the title compound as a yellow solid (62%).

m.p. 167-172° C.;

¹H NMR (200 MHz, DMSO) δ 0.79 (d, 3H, J=7.2 Hz, CH₃), 1.16-1.25 (m, 4H, CH &CH₃), 1.58 (m, 2H, CH₂), 2.12 (m, 2H, ArCH₂), 2.68 (m, 2H, CH₂Ar), 3.92 (m, 2H, NCH₂), 6.92 (s, 1H, ArH), 6.931 (s, 1H, ArH), 7.11-7.26 (m, 4H, ArH), 10.17 (s, 1H, NH);

MS (EI) m/e 404 [M⁺], 231, 174, 160.

### Preparation Example 3-40

### Preparation of 5,7-dichloro-3-naphthalen-1-ylmethyl-1H-quinazoline-2,4-dione (Intermediate II-40)

The same procedure as in Preparation Example 3-1 with the exception that Intermediate I-40 and triphosgene were used, was performed to give the title compound as a white solid (68%).

m.p. 274-276° C.:

¹H NMR (200 MHz, DMSO) δ 5.66 (s, 2H, NCH₂Ar), 7.17-7.20 (m, 2H, ArH), 7.25 (d, 1H, J=2.0 Hz, ArH), 7.33-7.41 (m, 1H, ArH), 7.47-7.61 (m, 2H, ArH), 7.73 (d, 1H, 7=8.1 Hz, ArH), 7.85 (d, 1H, J=7.3 Hz, ArH), 8.17 (d, 1H, J=7.7 Hz, ArH), 11.72 (s, 1H, NH);

MS (EI) m/e 370 [M⁺].

### Preparation Example 3-41

### Preparation of 5,7-dichloro-3-(5-methyl-furan-2-ylmethyl)-1H-quinazoline-2,4-dione (Intermediate II-41)

The same procedure as in Preparation Example 3-1 with the exception that Intermediate 1-41 and triphosgene were used, was performed to give the title compound as a white solid (51%).

m.p. 269-272° C.;

¹H NMR (200 MHz, DMSO) δ 2.20 (s, 3H, ArCH₃), 4.96 (s, 2H, NCH₂Ar), 5.96 (d, 1H, J=1.6 Hz, ArH), 6.17 (m, 1H, ArH), 7.16 (d, 1H, J=2.0 Hz, ArH), 7.38 (d, 1H, J=2.0 Hz, ArH), 11.72 (s, 1H, NH);

MS (EI) m/e 324 [M⁺], 253, 126, 95.

### Preparation Example 3-42

### Preparation of 5,7-dichloro-3-pyridin-4-ylmethyl-1H-quinazoline-2,4-dione (Intermediate II-42)

The same procedure as in Preparation Example 3-1 with the exception that Intermediate I-42 and triphosgene were used, was performed to give the title compound as a white solid (79%).

m.p. 354-364° C.;

¹H NMR (200 MHz, DMSO-d₆) δ 5.25 (s, 2H, NCH₂Ar), 7.28 (d, 1H, J=2.0 Hz, ArH), 7.42 (d, 1H, J=2.0 Hz, ArH), 7.94 (d, 2H, J=6.4 Hz, ArH), 8.79 (d, 2H, J=6.4 Hz, ArH), 11.97 (br s, 1H, NH);

MS (EI) m/e 321 [M⁺].

### Preparation Example 4

### Preparation of Intermediate II (Method B)

### Preparation Example 4-1

### Preparation of 3-benzyl-5,7-dibromo-1H-quinazoline-2,4-dione (Intermediate II-43)

To a solution of 2-amino-4,6-dibromo-benzoic acid methyl ester (0.5 g, 1.61 mmol) in CH₂Cl₂ (35 ml) was added benzyl isocyanate (0.26 g, 1.94 mmol) in a nitrogen atmosphere. The resulting solution was heated overnight. Following completion of the reaction, the solvent was evaporated in a vacuum. The residue was washed with diethylether and dried in a vacuum to 1 afford 2-(3-benzyl-ureido)-4,6-dibromo-benzoic acid methyl ester as a yellow solid (0.29 g, 41%).

m.p.>280° C.;

¹H NMR (200 MHz, CDCl₃+DMSO) δ 3.22 (m, 3H, OCH₃), 4.28 (m, 1H, NH), 5.05 (s, 2H, NCH₂Ar), 7.16-7.35 (m, 5H, ArH), 7.48 (s, 1H, ArH), 7.93 (s, 1H, ArH), 11.64 (s, 1H, NH).

To a solution of ureido-anthranilate (0.26 g, 0.58 mmol) in methanol (35 ml) was added to 10% sodium hydroxide (10 ml). The solution was continuously stirred in a steam bath until it was clear (approximately 10 min). The reaction was cooled to room temperature, suspended in distilled water (30 ml), and acidified with cone. HQ. The precipitates thus formed were filtered 1 and dried in a vacuum to give the title compound as an ivory solid (0.16 g, 70%).

m.p.>280° C.;

¹H NMR (200 MHz, DMSO-d₆) δ 5.03 (s, 2H, NCH₂Ar), 7.23-7.32 (m, 5H, ArH), 7.39 (d, 1H, J=1.6 Hz, ArH), 7.68 (d, 1H, J=1.6 Hz, ArH), 11.73 (s, 1H, NH);

MS (EI) m/e 410[M⁺].

### Preparation Example 4-2

### Preparation of 5,7-dibromo-3-(2-chloro-benzyl)-1H-quinazoline-2,4-dione (Intermediate II-44)

The title compound was prepared as an ivory solid (0.97 g, 62%) in a manner identical to that of Method B, with the exception that 2-chlorobenzyl isocyanate (0.59 g, 3.53 mmol; prepared by reacting 2-chlorobenzylamine (0.50 g, 3.53 mmol) and triphosgene (0.42 g, 1.41 mmol) in toluene under a flux condition) and 2-amino-4,6-dibromo-benzoic acid methyl ester (0.87 g, 2.82 mmol) were used, and 10% sodium hydroxide (10 ml) was employed for the cyclization of the intermediate ureido-anthranilate.

m.p. 133-136° C.;

¹H NMR (200 MHz, DMSO) δ 5.07 (s, 2H, NCH₂Ar), 7.08-7.75 (m, 4H, ArH), 7.85 (d, 1H, J=1.6 Hz, ArH), 8.13 (d, 1H, J=1.6 Hz, ArH), 11.87 (s, 1H, NH);

MS (EI) m/e 442[M⁺].

### Preparation Example 5

### Preparation of Intermediate III

### Preparation Example 5-1

### Preparation of 1-benzyl-5,7-dichloro-3-methyl-1H-quinazoline-2,4-dione (Intermediate III-1)

To a solution of Intermediate II-1 (1.0 mmol) in DMF (15 ml) were added benzyliodide (or benzylbromide) (1.2 mmol) and a base, such as K₂CO₃ (3.0 mmol) (or sodium hydride (1.3 mmol)). The resulting reaction mixture was stirred at 80-100° C. (in the case of sodium hydride, at room temperature) for 3 hours. The residue thus formed was dissolved in ethyl acetate and washed with 0.5 M HCl, distilled water and brine. The organic layer thus formed was dried over anhydrous magnesium sulfate, dried and concentrated in a vacuum. Purification by flash column chromatography (eluent; a mixture of n-hexane and ethyl acetate) gave the title compound as a white solid (95%).

m.p. 166-169° C.;

¹H NMR (200 MHz, CDCl₃) δ 3.52 (s, 3H, NCH₃), 5.35 (s, 2H, NCH₂Ar), 7.04 (d, 1H, J=2.0 Hz, ArH), 7.24 (d, 1H, J=2.0 Hz, ArH), 7.25-7.41 (m, 5H, ArH);

MS (EI) m/e 334 [M⁺], 321, 248, 228, 91.

### Preparation Example 5-2

### Preparation of 1-benzyl-5,7-dichloro-3-ethyl-1H-quinazoline-2,4-dione (Intermediate III-2)

The same procedure as in Preparation Example 5-1, with the exception that Intermediate II-2 was used, was repeated to afford the title compound as a white solid (92%).

m.p. 145-149° C.;

¹H NMR (200 MHz, CDCl₃) δ 1.29 (t, 3H, J=7.3 Hz, CH₃), 4.16 (q, 2H, J=7.3 Hz, NCH₂), 5.34 (s, 2H, NCH₂Ar), 7.03 (d, 1H, J=2.0 Hz, ArH), 7.21-7.37 (m, 6H, ArH);

MS (EI) m/e 348 [M⁺], 322, 257, 231, 158.

### Preparation Example 5-3

### 1-benzyl-5,7-dichloro-3-propyl-1H-quinazoline-2,4-dione (Intermediate III-3)

The same procedure as in Preparation Example 5-1 with the exception that Intermediate II-3 was used, was performed to afford the title compound as a white solid (88%).

m.p. 111-115° C.;

¹H NMR (200 MHz, CDCl₃) δ 0.96 (t, 3H, J=7.3 Hz, CH₃), 1.76 (m, 2H, CH₂), 4.08 (m, 2H, NCH₂), 5.33 (s, 2H, NCH₂Ar), 7.03 (d, 1H, J=2.0 Hz, ArH), 7.20-7.34 (m, 5H, ArH), 7.36 (d, 1H, J=2.0 Hz, ArH);

MS (EI) m/e 364 [M⁺], 91, 72.

### Preparation Example 5-4

### Preparation of 1-benzyl-3-butyl-5,7-dichloro-1H-quinazoline-2,4-dione (Intermediate III-4)

The same procedure as in Preparation Example 5-1 with the exception that Intermediate II-4 was used, was repeated to afford the title compound as a white solid (88%).

m.p. 101-109° C.;

¹H NMR (200 MHz, CDCl₃) δ 0.94 (t, 3H, J=13 Hz, CH₃), 1.43 (m, 2H, CH₂), 1.72 (m, 2H, CH₂), 4.07 (q, 2H, J=6.9 Hz, NCH₂), 5.33 (s, 2H, NCH₂Ar), 7.03 (d, 1H, J=2.0 Hz, ArH), 7.20-7.40 (m, 6H, ArH);

MS (EI) m/e 376 [M⁺], 320, 248, 158, 91.

### Preparation Example 5-5

### Preparation of 1-benzyl-5,7-dichloro-3-(3-methyl-butyl)-1H-quinazoline-2,4-dione (Intermediate III-5)

The same procedure as in Preparation Example 5-1 with the exception that Intermediate II-5 was used, was repeated to afford the title compound as a white solid (98%).

m.p. 89-92° C.;

¹H NMR (200 MHz, CDCl₃) δ 0.98 (d, 6H, J=5.6 Hz, CH₃×2), 1.55-1.71 (m, 3H, CH₂&CH), 4.12 (m, 2H, NCH₂), 5.33 (s, 2H, NCH₂Ar), 7.02 (d, 1H, J=2.0 Hz, ArH), 7.20-7.40 (m, 6H, ArH);

MS (EI) m/e 390 [M⁺], 359, 321, 299, 91, 85.

### Preparation Example 5-6

### Preparation of 1-benzyl-5,7-dichloro-3-cyclohexylmethyl-1H-quinazoline-2,4-dione (Intermediate III-6)

The same procedure as in Preparation Example 5-1 with the exception that Intermediate II-6 was used, was repeated to afford the title compound as a white solid (95%).

m.p. 147-149° C.;

¹H NMR (200 MHz, CDCl₃) δ 1.06-1.25 (m, 5H, 2×CH₂ and cyclohexyl [symbol] CH), 1.58-1.71 (m, 6H, 3×cyclohexyl [symbol] CH₂), 3.97 (d, 2H, J=7.4 Hz, NCH₂), 5.33 (s, 2H, NCH₂Ar), 7.03 (d, 1H, J=1.8 Hz, ArH), 7.20-7.40 (m, 6H, ArH);

MS (EI) m/e 416 [M⁺].

### Preparation Example 5-7

### Preparation of 1-benzyl-5,7-dichloro-3-octyl-1H-quinazoline-2,4-dione (Intermediate III-7)

The same procedure as in Preparation Example 5-1 with the exception that Intermediate II-7 was used, was repeated to afford the title compound as a white solid (95%).

m.p. 82-85° C.;

¹H NMR (200 MHz, CDCl₃) δ 0.85 (t, 3H, J=7.0 Hz, CH₃), 1.22-1.41 (m, 10H, 5×CH₂), 1.70 (m, 2H, CH₂), 4.08 (t, 2H, J=7.0 Hz, NCH₂), 5.33 (s, 2H, NCH₂Ar), 7.03 (d, 1H, J=1.8 Hz, ArH), 7.20-7.41 (m, 6H, ArH);

MS (EI) m/e 432 [M⁺], 361, 321, 248, 214, 126, 91.

### Preparation Example 5-8

### Preparation of 1-benzyl-5,7-dichloro-3-phenyl-1H-quinazoline-2,4-dione (Intermediate III-8)

The same procedure as in Preparation Example 5-1 with the exception that Intermediate II-8 was used, was repeated to afford the title compound as a white solid (96%).

m.p. 169-174° C.;

¹H NMR (200 MHz, CDCl₃) δ 5.36 (s, 2H, NCH₂Ar), 7.14 (d, 1H, J=2.0 Hz, ArH), 7.26-7.57 (m, 11H, ArH);

MS (EI) m/e 396[M⁺].

### Preparation Example 5-9

### Preparation of 1-benzyl-5,7-dichloro-3-(4-methoxy-phenyl)-1H-quinazoline-2,4-dione (Intermediate III-9)

The same procedure as in Preparation Example 5-1 with the exception that Intermediate II-9 was used, was repeated to afford the title compound as a bright yellow solid (78%).

m.p. 175-177° C.;

¹H NMR (200 MHz, CDCl₃) δ 3.86 (s, 3H, OCH₃), 5.23 (s, 2H, NCH₂Ar), 6.93 (d, 2H, J=4.2 Hz, ArH), 7.29-7.37 (m, 5H, ArH), 7.43-7.47 (m, 4H, ArH);

MS (EI) m/e 426[M⁺].

### Preparation Example 5-10

### Preparation of 1,3-dibenzyl-5,7-dichloro-1H-quinazoline-2,4-dione (Intermediate III-10)

The same procedure as in Preparation Example 5-1 with the exception that Intermediate II-10 was used, was repeated to afford the title compound as a white solid (87%).

m.p. 104-106° C.;

¹H NMR (200 MHz, CDCl₃) δ 5.29 (s, 2H, NCH₂Ar), 5.33 (s, 2H, NCH₂Ar), 7.02 (d, 1H, J=2.0 Hz, ArH), 7.19-7.36 (m, 6H, ArH), 7.54-7.58 (m, 4H, ArH);

MS (EI) m/e 412[M⁺].

### Preparation Example 5-11

### Preparation of 1-benzyl-5,7-dichloro-3-(2-fluoro-benzyl)-1H-quinazoline-2,4-dione (Intermediate III-11)

The same procedure as in Preparation Example 5-1 with the exception that Intermediate II-11 was used, was repeated to afford the title compound as a white solid (80%).

m.p. 126-130° C.;

¹H NMR (200 MHz, CDCl₃) δ 5.34 (s, 2H, NCH₂Ar), 5.39 (s, 2H, NCH₂Ar), 7.02 (d, 1H, J=1.6 Hz, ArH). 7.03-7.42 (m, 10H, ArH);

MS (EI) m/e 428[M⁺].

### Preparation Example 5-12

### Preparation of 1-benzyl-5,7-dichloro-3-(3-fluoro-benzyl)-1H-quinazoline-2,4-dione (Intermediate III-12)

The same procedure as in Preparation Example 5-1 with the exception that Intermediate II-12 was used, was repeated to afford the title compound as a white solid (91%).

m.p. 112-116° C.;

¹H NMR (200 MHz, CDCl₃) δ 5.27 (s, 2H, NCH₂Ar), 5.33 (s, 2H, NCH₂Ar), 6.96 (m, 1H, ArH), 7.04 (d, 1H, J=1.6 Hz, ArH), 7.19-7.36 (m, 9H, ArH);

MS (EI) m/e 427[M⁺].

### Preparation Example 5-13

### Preparation of 1-benzyl-5,7-dichloro-3-(4-fluoro-benzyl)-1H-quinazoline-2,4-dione (Intermediate III-13)

The same procedure as in Preparation Example 5-1 with the exception that Intermediate II-13 was used, was repeated to afford the title compound as a white solid (93%).

m.p. 148-151° C.;

¹H NMR (200 MHz, CDCl₃) δ 5.25 (s, 2H, NCH₂Ar), 5.33 (s, 2H, NCH₂Ar), 6.96-7.60 (m, 11H, ArH);

MS (EI) m/e 427[M⁺].

### Preparation Example 5-14

### Preparation of 1-benzyl-5,7-dichloro-3-(2-chloro-benzyl)-1H-quinazoline-2,4-dione (Intermediate III-14)

The same procedure as in Preparation Example 5-1 with the exception that Intermediate II-14 was used, was repeated to afford the title compound as a white solid (97%).

m.p. 197-200° C.;

¹H NMR (200 MHz, CDCl₃) δ 5.36 (s, 2H, NCH₂Ar), 5.43 (s, 2H, NCH₂Ar), 7.09 (d, 2H, J=1.2 Hz, ArH), 7.15-7.20 (m, 5H, ArH), 7.27-7.41 (m, 4H, ArH);

MS (EI) m/e 445 [M⁺]

### Preparation Example 5-15

### Preparation of 1-benzyl-5,7-dichloro-3-(3-chloro-benzyl)-1H-quinazoline-2,4-dione (Intermediate III-15)

The same procedure as in Preparation Example 5-1 with the exception that Intermediate II-15 was used, was repeated to afford the title compound as a white solid (92%).

m.p. 123-129° C.;

¹H NMR (200 MHz, CDCl₃) δ 5.26 (s, 2H, NCH₂Ar), 5.34 (s, 2H, NCH₂Ar), 7.05 (d, 1H, J=1.6 Hz, ArH), 7.20-7.52 (m, 9H, ArH), 7.52 (d, 1H, J=1.6 Hz, ArH);

MS (EI) m/e 444 [M⁺]

### Preparation Example 5-16

### Preparation of 1-benzyl-5,7-dichloro-3-(4-chloro-benzyl)-1H-quinazoline-2,4-dione (Intermediate III-16)

The same procedure as in Preparation Example 5-1 with the exception that Intermediate II-16 was used, was repeated to afford the title compound as a white solid (93%).

m.p. 161-165° C.;

¹H NMR (200 MHz, CDCl₃) δ 5.25 (s, 2H, NCH₂Ar), 5.33 (s, 2H, NCH₂Ar), 7.04 (d, 1H, J=1.6 Hz, ArH), 7.18-7.24 (m, 4H, ArH), 7.27 (d, 1H, J=1.6 Hz, ArH), 7.13-7.36 (m, 3H, ArH), 7.53 (d, 2H, J=8.0 Hz, ArH);

MS (EI) m/e 444[M⁺].

### Preparation Example 5-17

### Preparation of 1-benzyl-3-(2-bromo-benzyl)-5,7-dichloro-1H-quinazoline-2,4-dione (Intermediate III-17)

The same procedure as in Preparation Example 5-1 with the exception that Intermediate II-17 was used, was repeated to afford the title compound as a white solid (67%).

m.p. 129-132° C.;

¹H NMR (200 MHz, CDCl₃) δ 5.38 (s, 2H, NCH₂Ar), 5.43 (s, 2H, NCH₂Ar), 7.05 (d, 1H, J=7.6 Hz, ArH), 7.12-7.43 (m, 9H, ArH), 7.63 (d, 1H, J=7.6 Hz, ArH);

MS (EI) m/e 488 [M⁺].

### Preparation Example 5-18

### Preparation of 1-benzyl-3-(3-bromo-benzyl)-5,7-dichloro-1H-quinazoline-2,4-dione

(Intermediate III-18)

The same procedure as in Preparation Example 5-1 with the exception that Intermediate II-18 was used, was repeated to afford the title compound as a white solid (94%).

m.p. 121-128° C.;

¹H NMR (200 MHz, CDCl₃) δ 5.25 (s, 2H, NCH₂Ar), 5.34 (s, 2H, NCH₂Ar), 7.05 (d, 1H, J=1.8 Hz, ArH), 7.20-7.50 (m, 9H, ArH), 7.80 (m, 1H, ArH);

MS (EI) m/e 488 [M⁺], 399.

### Preparation Example 5-19

### Preparation of 1-benzyl-3-(4-bromo-benzyl)-5,7-dichloro-1H-quinazoline-2,4-dione (Intermediate III-19)

The same procedure as in Preparation Example 5-1 with the exception that Intermediate II-19 was used, was repeated to afford the title compound as a pale yellow solid (96%).

m.p. 163-168° C.;

¹H NMR (200 MHz, CDCl₃) δ 5.23 (s, 2H, NCH₂Ar), 5.33 (s, 2H, NCH₂Ar), 7.04 (d, 2H, J=2.0 Hz, ArH), 7.18-7.27 (m, 3H, ArH), 7.32-7.37 (m, 3H, ArH), 7.45-7.46 (m, 3H, ArH);

MS (EI) m/e 488 [M⁺].

### Preparation Example 5-20

### Preparation of 1-benzyl-5,7-dichloro-3-(3-iodo-benzyl)-1H-quinazoline-2,4-dione (Intermediate III-20)

The same procedure as in Preparation Example 5-1 with the exception that Intermediate II-20 was used, was repeated to afford the title compound as a pale yellow solid (99%).

m.p. 155-157° C.;

¹H NMR (200 MHz, CDCl₃) δ 5.22 (s, 2H, NCH₂Ar), 5.34 (s, 2H, NCH₂Ar), 7.02-7.08 (m, 2H, ArH), 7.20-7.40 (m, 7H, ArH), 7.49 (d, 1H, J=7.8 Hz, ArH), 7.60 (d, 1H, J=8.0 Hz, ArH), 7.87 (s, 1H, ArH);

MS (EI) m/e 536 [M⁺].

### Preparation Example 5-21

### Preparation of 1-benzyl-5,7-dichloro-3-(4-iodo-benzyl)-1H-quinazoline-2,4-dione (Intermediate III-21)

The same procedure as in Preparation Example 5-1 with the exception that Intermediate II-21 was used, was repeated to afford the title compound as a pale yellow solid (84%).

m.p. 181-184° C.;

¹H NMR (200 MHz, CDCl₃) δ 5.22 (s, 2H, NCH₂Ar), 5.32 (s, 2H, NCH₂Ar), 7.03 (d, 1H, J=1.6 Hz, ArH), 7.18-7.36 (m, 8H, ArH), 7.63 (d, 2H, J=8.2 Hz, ArH);

MS (EI) m/e 536 [M⁺].

### Preparation Example 5-22

### Preparation of 1-benzyl-5,7-dichloro-3-(2-methyl-benzyl)-1H-quinazoline-2,4-dione (Intermediate III-22)

The same procedure as in Preparation Example 5-1 with the exception that Intermediate II-22 was used, was repeated to afford the title compound as a white solid (98%).

m.p. 139-145° C.;

¹H NMR (200 MHz, CDCl₃) δ 2.50 (s, 3H, CH₃), 5.34 (s, 2H, NCH₂Ar), 5.37 (s, 2H, NCH₂Ar), 7.10 (d, 1H, J=1.6 Hz, ArH), 7.12-7.39 (m, 10H, ArH);

MS (EI) m/e 424[M⁺].

### Preparation Example 5-23

### Preparation of 1-benzyl-5,7-dichloro-3-(3-methyl-benzyl)-1H-quinazoline-2,4-dione (Intermediate III-23)

The same procedure as in Preparation Example 5-1 with the exception that Intermediate II-23 was used, was repeated to afford the title compound as a white solid (95%).

m.p. 111-114° C.;

¹H NMR (200 MHz, CDCl₃) δ 2.33 (s, 3H, CH₃), 5.26 (s, 2H, NCH₂Ar), 5.33 (s, 2H, NCH₂Ar), 7.03 (d, 1H, J=1.2 Hz, ArH), 7.08 (m, 1H, ArH), 7.17-7.29 (m, 4H, ArH), 7.23-7.35 (m, 5H, ArH);

MS (EI) m/e 424[M⁺]

### Preparation Example 5-24

### Preparation of 1-benzyl-5,7-dichloro-3-(4-methyl-benzyl)-1H-quinazoline-2,4-dione (Intermediate III-24)

The same procedure as in Preparation Example 5-1 with the exception that Intermediate II-24 was used, was repeated to afford the title compound as a white solid (88%).

m.p. 166-169° C.;

¹H NMR (200 MHz, CDCl₃) δ 2.34 (s, 3H, CH₃), 5.28 (s, 2H, NCH₂Ar), 5.34 (s, 2H, NCH₂Ar), 7.05 (d, 1H, J=1.6 Hz, ArH), 7.21 (d, 1H, J=7.6 Hz, ArH), 7.25 (d, 1H, J=1.6 Hz, ArH), 7.28-7.41 (m, 5H, ArH), 7.51 (d, 2H, J=8.2 Hz, ArH);

MS (EI) m/e 424 [M⁺]

### Preparation Example 5-25

### Preparation of 1-benzyl-5,7-dichloro-3-(2-methoxy-benzyl)-1H-quinazoline-2,4-dione (Intermediate III-25)

The same procedure as in Preparation Example 5-1 with the exception that Intermediate II-25 was used, was repeated to afford the title compound as a white solid (88%).

m.p. 195-197° C.;

¹H NMR (200 MHz, CDCl₃) δ 3.85 (s, 3H, OCH₃), 5.36 (s, 2H, NCH₂Ar), 5.32 (s, 2H, NCH₂Ar), 6.89 (d, 2H, J=7.8 Hz, ArH), 7.06 (d, 2H, J=7.8 Hz, ArH), 7.24-7.35 (m, 7H, ArH);

MS (EI) m/e 440[M⁺].

### Preparation Example 5-26

### Preparation of 1-benzyl-5,7-dichloro-3-(3-methoxy-benzyl)-1H-quinazoline-2,4-dione (Intermediate III-26)

The same procedure as in Preparation Example 5-1 with the exception that Intermediate II-26 was used, was repeated to afford the title compound as a white solid (88%).

m.p. 106-108° C.;

¹H NMR (200 MHz, CDCl₃) δ 3.80 (m, 3H, OCH₃), 5.30 (s, 2H, NCH₂Ar), 5.34 (s, 2H, NCH₂Ar), 6.85 (d, 1H, J=2.0 Hz, ArH), 7.04 (d, 1H, J=2.0 Hz, ArH), 7.10-7.36 (m, 9H, ArH);

MS (EI) m/e 440 [M⁺].

### Preparation Example 5-27

### Preparation of 1-benzyl-5,7-dichloro-3-(4-methoxy-benzyl)-1H-quinazoline-2,4-dione (Intermediate III-27)

The same procedure as in Preparation Example 5-1 with the exception that Intermediate II-27 was used, was repeated to afford the title compound as a white solid (96%).

m.p. 138-143° C.;

¹H NMR (200 MHz, CDCl₃) δ 3.78 (s, 3H, OCH₃), 5.23 (s, 2H, NCH₂Ar), 5.32 (s, 2H, NCH₂Ar), 6.87 (d, 2H, J=8.6 Hz, ArH), 7.01-7.34 (m, 7H, ArH), 7.56 (d, 2H, J=8.6 Hz, ArH);

MS (EI) m/e 440 [M⁺], 349 [M⁺-C₇H₇], 319.

### Preparation Example 5-28

### Preparation of 1-benzyl-5,7-dichloro-3-(2-nitro-benzyl)-1H-quinazoline-2,4-dione (Intermediate III-28)

The same procedure as in Preparation Example 5-1 with the exception that Intermediate II-28 was used, was repeated to afford the title compound as a white solid (66%).

m.p. 239-242° C.;

¹H NMR (200 MHz, DMSO-d₆) δ 5.42 (m, 4H, NCH₂Ar×2), 7.23-7.36 (m, 6H, ArH), 7.49-7.72 (m, 4H, ArH), 8.08 (d, 1H, J=6.6 Hz, ArH);

MS (EI) m/e 455 [M⁺].

### Preparation Example 5-29

### Preparation of 1-benzyl-5,7-dichloro-3-(3-nitro-benzyl)-1H-quinazoline-2,4-dione (Intermediate III-29)

The same procedure as in Preparation Example 5-1 with the exception that Intermediate II-29 was used, was repeated to afford the title compound as a white solid (72%).

m.p. 243-248° C.;

¹H NMR (200 MHz, DMSO-d*) δ 5.26 (S, 2H, NCH₂Ar), 5.42 (s, 2H, NCH₂Ar), 7.26-7.33 (m, 6H, ArH), 7.50 (d, 1H, J=1.6 Hz, ArH), 7.60 (dd, 1H, J=8.0, 8.2 Hz, ArH), 7.84 (d, 1H, J=8.6 Hz, ArH), 8.12 (d, 1H, J=7.2 Hz, ArH), 8.24 (s, 1H, ArH);

MS (EI) m/e 455[M⁺].

### Preparation Example 5-30

### Preparation of 1-benzyl-5,7-dichloro-3-(4-nitro-benzyl)-1H-quinazoline-2,4-dione (Intermediate III-30)

The same procedure as in Preparation Example 5-1 with the exception that Intermediate II-30 was used, was repeated to afford the title compound as a white solid (91%).

m.p. 190-193° C.;

¹H NMR (200 MHz, CDCl₃) δ 5.34 (s, 2H, NCH₂Ar), 5.36 (s, 2H, NCH₂Ar), 7.07 (d, 1H, J=1.6 Hz, ArH), 7.23-7.37 (m, 5H, ArH), 7.72 (m, 2H, ArH), 8.21 (m, 2H, ArH);

MS (EI) m/e 455 [M⁺].

### Preparation Example 5-31

### Preparation of 4-(1-benzyl-5,7-dichloro-2,4-dioxo-1,4-dihydro-2H-quinazoline-3-ylmethyl (Intermediate III-31)

The same procedure as in Preparation Example 5-1 with the exception that Intermediate II-31 was used, was repeated to afford the title compound as a yellow solid (97%).

m.p. 216-220° C.;

¹H NMR (200 MHz, CDCl₃) δ 5.30-5.33 (m, 4H, NCH₂Ar×2), 7.06 (d, 1H, J=2.2 Hz, ArH), 7.18-7.40 (m, 6H, ArH), 7.63 (m, 4H, ArH);

MS (EI) m/e 435 [M⁺].

### Preparation Example 5-32

### Preparation of 4-(1-benzyl-5,7-dichloro-2,4-dioxo-1,4-dihydro-2H-quinazoline-3-ylmethyl)-benzoic acid methyl ester(Intermediate III-32)

The same procedure as in Preparation Example 5-1 with the exception that Intermediate II-32 was used, was repeated to afford the title compound as a yellow solid (97%).

m.p. 153-156° C.;

¹H NMR (200 MHz, CDCl₃) δ 3.88-3.90 (m, 3H, OMe), 5.33 (m, 4H, NCH₂Ar×2), 7.04-8.01 (m, 11H, ArH);

MS (EI) m/e 468 [M⁺].

### Preparation Example 5-33

### Preparation of 1-benzyl-5,7-dichloro-3-(3,4-dimethyl-benzyl)-1H-quinazoline-2,4-dione (Intermediate III-33)

The same procedure as in Preparation Example 5-1 with the exception that Intermediate II-33 was used, was repeated to afford the title compound as a dark yellow solid (97%).

m.p. 126-132° C.;

¹H NMR (200 MHz, CDCl₃) δ 2.22 (s, 3H, ArCH₃), 2.24 (s, 3H, ArCH₃), 5.23 (s, 2H, NCH₂Ar), 5.32 (s, 2H, NCH₂Ar), 7.01 (d, 1H, J=1.8 Hz, ArH), 7.06-7.38 (m, 9H, ArH);

MS (EI) m/e 438 [M⁺].

### Preparation Example 5-34

### Preparation of 1-benzyl-5,7-dichloro-3-[(R)-1-phenyl-ethyl]-1H-quinazoline-2,4-dione (Intermediate III-34)

The same procedure as in Preparation Example 5-1 with the exception that Intermediate II-34 was used, was repeated to afford the title compound as a yellow solid (66%).

m.p. 96-100° C.;

¹H NMR (200 MHz, CDCl₃) δ 1.99 (d, 3H, J=7.0 Hz, CH₃), 5.29 (d, 2H, J=5.8 Hz, NCH₂Ar), 6.49 (q, 1H, J=7.4 Hz, NCH), 7.02 (d, 1H, J=2.0 Hz, ArH), 7.15-7.40 (m, 4H, ArH), 7.50-7.54 (m, 2H, ArH);

MS (EI) m/e 425[M⁺].

### Preparation Example 5-35

### Preparation of 1-benzyl-5,7-dichloro-3-[(S)-1-phenyl-ethyl]-1H-quinazoline-2,4-dione (Intermediate 1H-35)

The same procedure as in Preparation Example 5-1 with the exception that Intermediate II-35 was used, was repeated to afford the title compound as a yellow solid (88%).

m.p. 93-97° C.;

¹H NMR (200 MHz, CDCl₃) δ 2.01 (d, 3H, J=7.4 Hz, CH₃), 5.30 (d, 2H, J=6.0 Hz, NCH₂Ar), 6.50 (q, 1H, J=7.4 Hz, NCH), 7.03 (d, 1H, J=2.0 Hz, ArH), 7.18-7.22 (m, 2H, ArH), 7.24-7.41 (m, 6H, ArH), 7.25 (d, 1H, J=2.0 Hz, ArH), 7.52-7.56 (m, 2H, ArH);

MS (EI) m/e 425 [M⁺].

### Preparation Example 5-36

### Preparation of 1-benzyl-5,7-dichloro-3-phenethyl-1H-quinazoline-2,4-dione (Intermediate III-36)

The same procedure as in Preparation Example 5-1 with the exception that Intermediate II-36 was used, was repeated to afford the title compound as a white solid (73%).

m.p. 169-171° C.;

¹H NMR (200 MHz, CDCl₃) δ 3.04 (m, 2H, CH₂Ar), 4.34 (m, 2H, NCH₂), 5.31 (s, 2H, NCH₂Ar), 7.02-7.03 (m, 12H, ArH);

MS (EI) m/e 424 [M⁺], 320, 104, 91.

### Preparation Example 5-37

### Preparation of 1-benzyl-5,7-dichloro-3-(3-phenyl-propyl)-1H-quinazoline-2,4-dione (Intermediate III-37)

The same procedure as in Preparation Example 5-1 with the exception that Intermediate II-37 was used, was repeated to afford the title compound as a white solid (64%).

m.p. 198-202° C.;

¹H NMR (200 MHz, CDCl₃) δ 2.04 (m, 2H, CH₂), 2.75 (t, 2H, J=7.5 Hz, CH₂Ar), 4.17 (t, 2H, J=7.5 Hz, NCH₂), 5.30 (s, 2H, NCH₂Ar), 7.00-7.40 (m, 12H, ArH);

MS (EI) m/e 439 [M⁺], 334, 321.

### Preparation Example 5-38

### Preparation of 1-benzyl-5,7-dichloro-3-[3-(3,5-dimethyl-phenyl)-propyl]-1H-quinazoline-2,4-dione (Intermediate III-38)

The same procedure as in Preparation Example 5-1 with the exception that Intermediate II-38 was used, was repeated to afford the title compound as a white solid (94%).

m.p. 144-147° C.;

¹H NMR (200 MHz, CDCl₃) δ 2.05 (m, 2H, CH₂), 2.24 (s, 6H, ArCH₃×2), 2.62 (t, 2H, J=1.1 Hz, CH₂Ar), 4.13 (t, 2H, J=1.1 Hz, NCH₂), 5.29 (s, 2H, NCH₂Ar), 6.78 (s, 1H, ArH), 6.83 (s, 2H, ArH), 7.00 (d, 1H, J=2.0 Hz, ArH), 7.22-7.35 (m, 6H, ArH);

MS (EI) m/e 466 [M⁺], 375, 321, 230, 146, 91.

### Preparation Example 5-39

### Preparation of 1-benzyl-5,7-dichloro-3-[3-(3-isobutyl-phenyl)-propyl]-1H-quinazoline-2,4-dione (Intermediate III-39)

The same procedure as in Preparation Example 5-1 with the exception that Intermediate II-39 was used, was repeated to afford the title compound as a pale yellow solid (97%).

m.p. 108-111° C.;

¹H NMR (200 MHz, CDCl₃) δ 0.85 (d, 3H, J=6.8 Hz, CH₃), 1.09-1.35 (m, 4H, CH& CH₃), 1.62 (m, 2H, CH₂), 2.05 (m, 2H, CH₂), 2.66 (m, 2H, CH₂Ar), 4.10 (m, 2H, NCH₂), 5.26 (m, 2H, NCH₂Ar), 6.95 (s, 1H, ArH), 6.96 (s, 1H, ArH), 7.01-7.39 (m, 9H, ArH);

MS (EI) m/e 494[M⁺], 403, 321, 174, 91.

### Preparation Example 5-40

### Preparation of 1-benzyl-5,7-dichloro-3-naphthalen-1-ylmethyl-1H-quinazoline-2,4-dione (Intermediate III-40)

The same procedure as in Preparation Example 5-1 with the exception that Intermediate II-40 was used, was repeated to afford the title compound as a white solid (97%).

m.p. 220-226° C.;

¹H NMR (200 MHz, CDCl₃) δ 5.37 (s, 2H, NCH₂Ar), 5.83 (s, 2H, NCH₂Ar), 7.08 (d, 1H, J=1.6 Hz, ArH), 7.22-7.58 (m, 10H, ArH), 7.78 (m, 1H, ArH), 7.85 (d, 1H, J=7.7 Hz, ArH), 8.26 (d, 1H, J=7.7 Hz, ArH);

MS (EI) m/e 460 [M⁺], 319, 141, 91.

### Preparation Example 5-41

### Preparation of 1-benzyl-5,7-dichloro-3-(5-methyl-furan-2-ylmethyl)-1H-quinazoline-2,4-dione (Intermediate III-41)

The same procedure as in Preparation Example 5-1 with the exception that Intermediate II-41 was used, was repeated to afford the title compound as a white solid (97%).

m.p. 159-161° C.;

¹H NMR (200 MHz, CDCl₃) δ 2.26 (s, 3H, ArCH₃), 5.26 (s, 2H, NCH₂Ar), 5.35 (s, 2H, NCH₂Ar), 5.89 (d, 1H, J=3.0 Hz, ArH), 6.32 (d, 1H, J=3.0 Hz, ArH), 7.03 (d, 1H, J=1.8 Hz, ArH), 7.22 (d, 1H, J=1.8 Hz, ArH), 7.24-7.36 (m, 5H, ArH);

MS (EI) m/e 414 [M⁺], 320, 248, 158, 95, 91.

### Preparation Example 5-42

### Preparation of 1-benzyl-5,7-dichloro-3-pyridin-4-ylmethyl-1H-quinazoline-2,4-dione (Intermediate III-42)

The same procedure as in Preparation Example 5-1 with the exception that Intermediate II-42 was used, was repeated to afford the title compound as a yellow solid (24%).

m.p. 174-176° C.;

¹H NMR (200 MHz, CDCl₃) δ 5.28 (s, 2H, NCH₂Ar), 5.34 (s, 2H, NCH₂Ar), 7.07 (d, 1H, J=1.6 Hz, ArH), 7.19-7.39 (m, 10H, ArH);

MS (EI) m/e 411 [M⁺].

### Preparation Example 5-43

### Preparation of 3-benzyl-5,7-dichloro-1-methyl-1H-quinazoline-2,4-dione (Intermediate III-43)

The same procedure as in Preparation Example 5-1 with the exception that Intermediate II-10 and methyl iodide (or methyl bromide) were used, was repeated to afford the title compound as a white solid (79%).

m.p. 173-176° C.;

¹H NMR (200 MHz, CDCl₃+CD₃OD) δ 3.40 (s, 3H, NCH₃), 5.23 (s, 2H, NCH₂Ar), 7.17 (d, 1H, J=1.6 Hz, ArH), 7.28-7.52 (m, 6H, ArH);

MS (EI) m/e 334 [M⁺], 243, 91, 77.

### Preparation Example 5-44

### Preparation of 3-benzyl-5,7-dichloro-1-ethyl-1H-quinazoline-2,4-dione (Intermediate III-44)

The same procedure as in Preparation Example 5-1 with the exception that Intermediate II-10 and ethyl iodide (or ethyl bromide) were used, was repeated to afford the title compound as a white solid (96%).

m.p. 78-82° C.;

¹H NMR (200 MHz, CDCl₃) δ 1.30 (t, 3H, J=6.9 Hz, CH₃), 4.10 (q, 2H, J=6.9 Hz, NCH₂), 5.22 (s, 2H, NCH₂Ar), 7.11 (d, 1H, J=1.6 Hz, ArH), 7.27-7.55 (m, 6H, ArH);

MS (EI) m/e 348 [M⁺], 320, 278, 91, 77.

### Preparation Example 5-45

### Preparation of 3-benzyl-5,7-dichloro-1-propyl-1H-quinazoline-2,4-dione (Intermediate III-45)

The same procedure as in Preparation Example 5-1 with the exception that Intermediate II-10 and propyl iodide (or propyl bromide) were used, was repeated to afford the title compound as a pale yellow solid (98%).

m.p. 90-93° C.;

¹H NMR (200 MHz, CDCl₃) δ 0.99 (t, 3H, J=7.1 Hz, CH₃), 1.74 (m, 2H, CH₂), 4.00 (t, 2H, J=7.7 Hz, NCH₂), 5.23 (s, 2H, NCH₂Ar), 7.07 (d, 1H, J=1.6 Hz, ArH), 7.26-7.54 (m, 6H, ArH); MS (EI) m/e 361 [M⁺], 320.

### Preparation Example 5-46

### Preparation of 3-benzyl-1-butyl-5,7-dichloro-1H-quinazoline-2,4-dione (Intermediate III-46)

The same procedure as in Preparation Example 5-1 with the exception that Intermediate II-10 and butyl iodide (or butyl bromide) were used, was repeated to afford the title compound as a bright yellow solid (98%).

m.p. 108-110° C.;

¹H NMR (200 MHz, CDCl₃) δ 0.96 (t, 3H, J=7.1 Hz, CH₃) 1.44 (m, 2H, CH₂), 1.66 (m, 2H, CH₂) 4.03 (t, 2H, J=7.6 Hz, NCH₂), 5.23 (s, 2H, NCH₂Ar), 7.07 (d, 1H, J=2.0 Hz, ArH), 7.26-7.54 (m, 6H, ArH);

MS (EI) m/e 376 [M⁺], 320, 91.

### Preparation Example 5-47

### Preparation of 1,3-dibenzyl-5,7-dibromo-1H-quinazoline-2,4-dione (Intermediate III-47)

The same procedure as in Preparation Example 5-1 with the exception that Intermediate II-43 were used, was repeated to afford the title compound as a white solid (88%).

m.p. 169-172° C.;

¹H NMR (200 MHz, CDCl₃) δ 5.30 (s, 2H, NCH₂Ar), 5.33 (s, 2H, NCH₂Ar), 7.19-7.36 (m, 1 OH, ArH), 7.57 (d, 1H, J=1.8 Hz, ArH), 7.64 (d, 1H, J=1.8 Hz, ArH);

MS (EI) m/e 500 [M⁺].

### Preparation Example 5-48

### Preparation of 1-benzyl-5,7-dibromo-3-(2-chloro-benzyl)-1H-quinazoline-2,4-dione (Intermediate III-48)

The same procedure as in Preparation Example 5-1 with the exception that Intermediate II-44 was used, was repeated to afford the title compound as a white solid (77%).

m.p. 202-205° C.;

¹H NMR (200 MHz, CDCl₃) δ 5.36 (s, 2H, NCH₂Ar), 5.44 (s, 2H, NCH₂Ar), 7.04-7.42 (m, 9H, ArH), 7.64-7.70 (m, 2H, ArH);

MS (EI) m/e 534 [M⁺].

The structural formulae of the Intermediate I-III compounds prepared in Preparation Examples 2˜5 are given in Table 1, below.

TABLE 1

| Int. | Structure |
|---|---|
| I-1 | 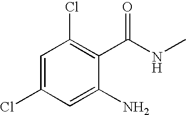 |
| I-2 | 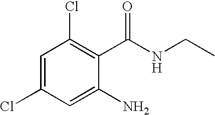 |
| I-3 | 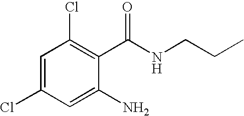 |
| I-4 | 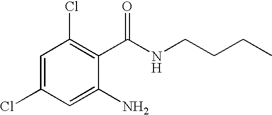 |
| I-5 | 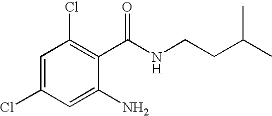 |
| I-6 | 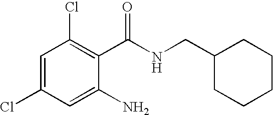 |
| I-7 | 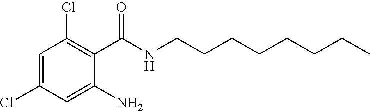 |
| I-8 | 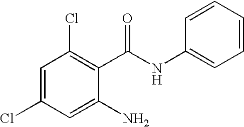 |
| I-9 | 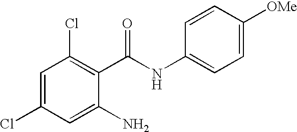 |
| I-10 | 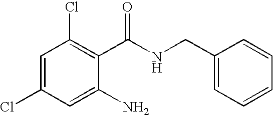 |
| I-11 | 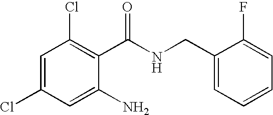 |
| I-12 | 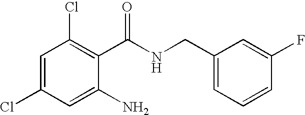 |
| I-13 | 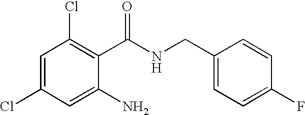 |
| I-14 | 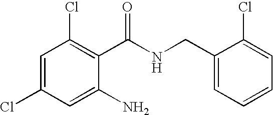 |
| I-15 | 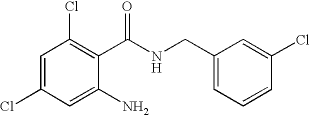 |
| I-16 | 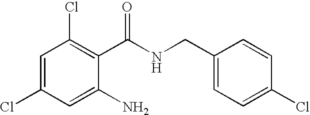 |
| I-17 | 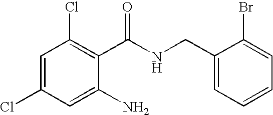 |
| I-18 | 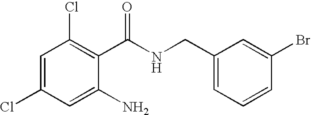 |
| I-19 | 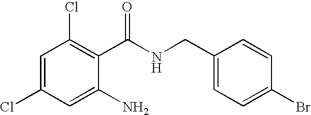 |
| I-20 | 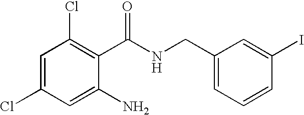 |
| I-21 | 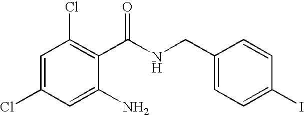 |
| I-22 | 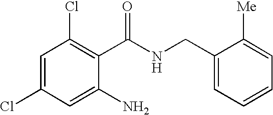 |
| I-23 | 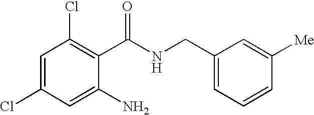 |
| I-24 | 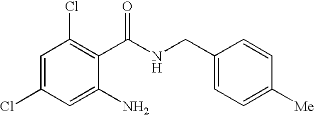 |
| I-25 | 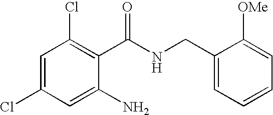 |
| I-26 | 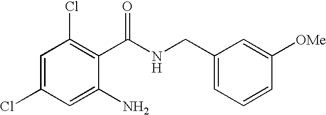 |
| I-27 | 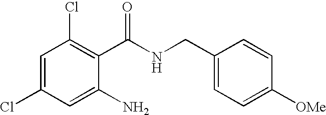 |
| I-28 | 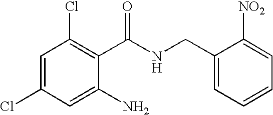 |
| I-29 | 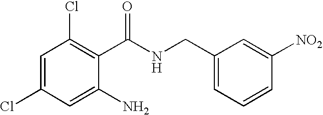 |
| I-30 | 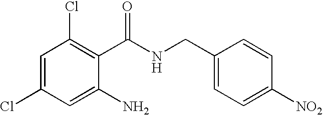 |
| I-31 | 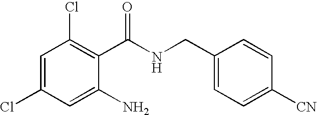 |
| I-32 | 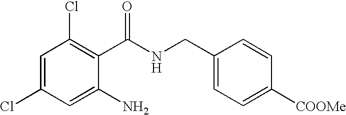 |
| I-33 | 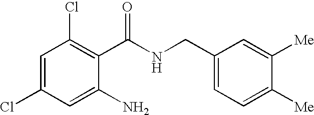 |
| I-34 | 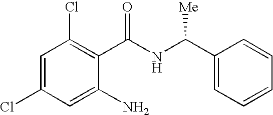 |
| I-35 | 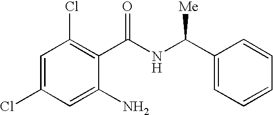 |
| I-36 | 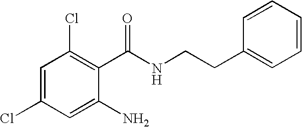 |
| I-37 | 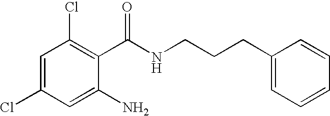 |
| I-38 | 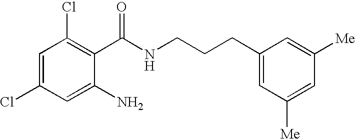 |
| I-39 | 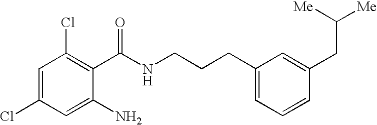 |
| I-40 | 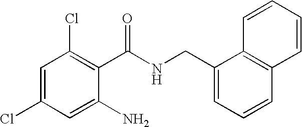 |
| I-41 | 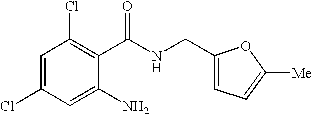 |
| I-42 | 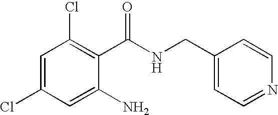 |
| II-1 | 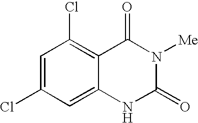 |
| II-2 | 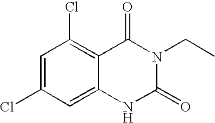 |
| II-3 | 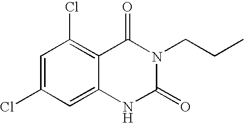 |
| II-4 | 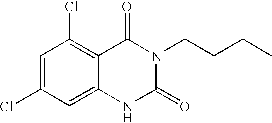 |
| II-5 | 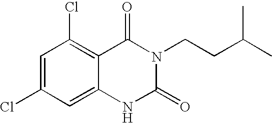 |
| II-6 | 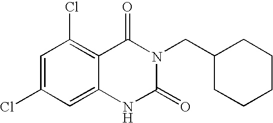 |
| II-7 | 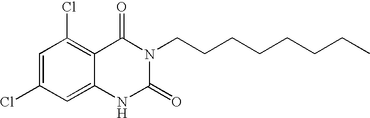 |
| II-8 | 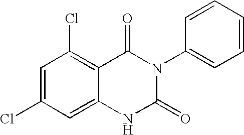 |
| II-9 | 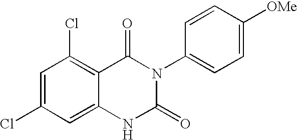 |
| II-10 | 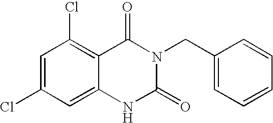 |
| II-11 | 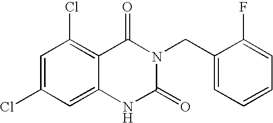 |
| II-12 | 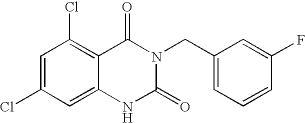 |
| II-13 | 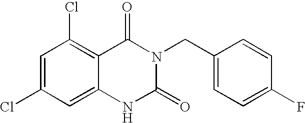 |
| II-14 | 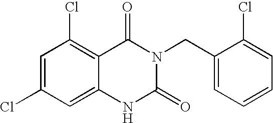 |
| II-15 | 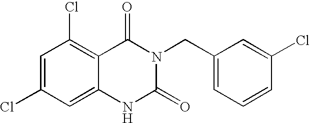 |
| II-16 | 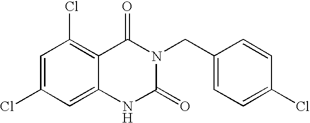 |
| II-17 | 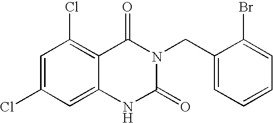 |
| II-18 | 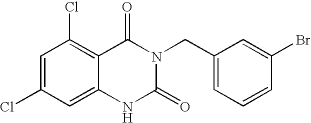 |
| II-19 | 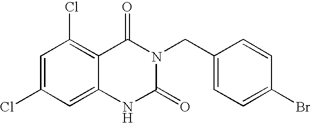 |
| II-20 | 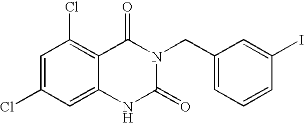 |
| II-21 | 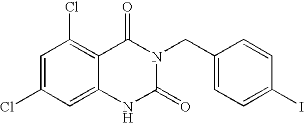 |
| II-22 | 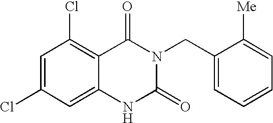 |
| II-23 | 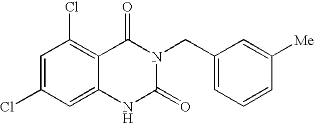 |
| II-24 | 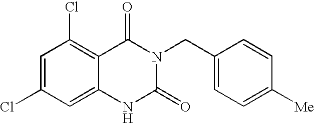 |
| II-25 | 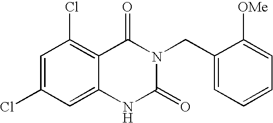 |
| II-26 | 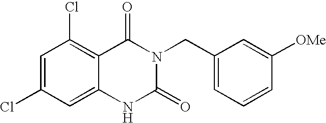 |
| II-27 | 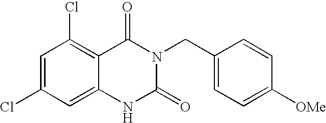 |
| II-28 | 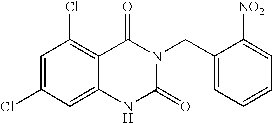 |
| II-29 | 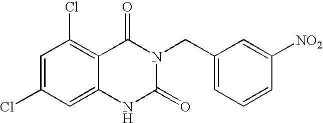 |
| II-30 | 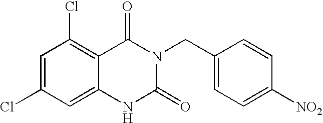 |
| II-31 | 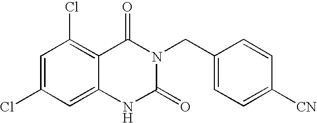 |
| II-32 | 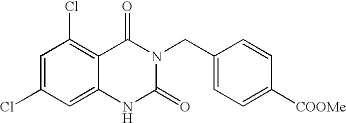 |
| II-33 | 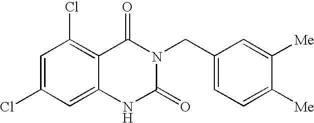 |
| II-34 | 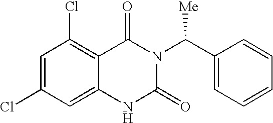 |
| II-35 | 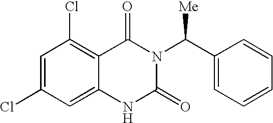 |
| II-36 | 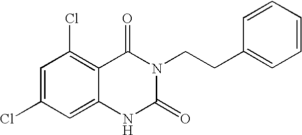 |
| II-37 | 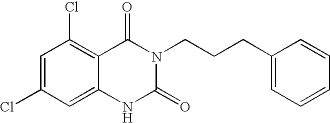 |
| II-38 | 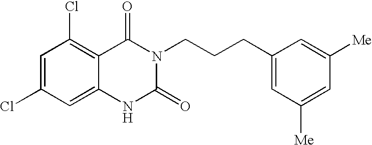 |
| II-39 | 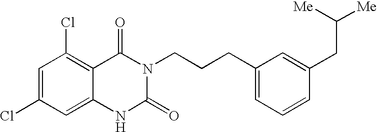 |
| II-40 | 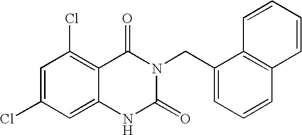 |
| II-41 | 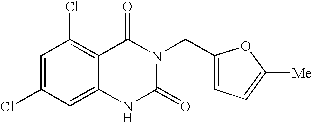 |
| II-42 | 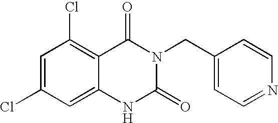 |
| II-43 | 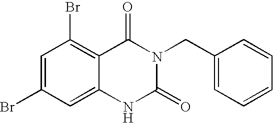 |
| II-44 | 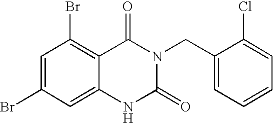 |
| III-1 | 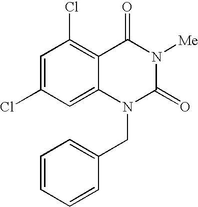 |
| III-2 | 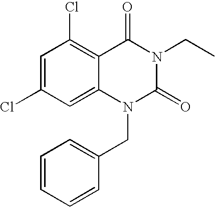 |
| III-3 | 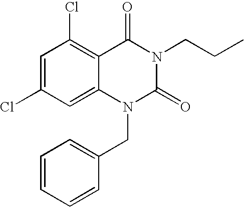 |
| III-4 | 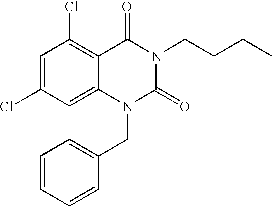 |
| III-5 | 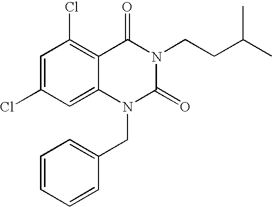 |
| III-6 | 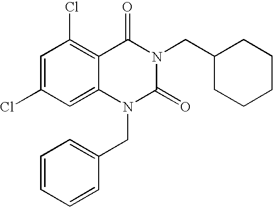 |
| III-7 | 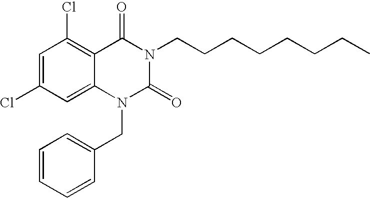 |
| III-8 | 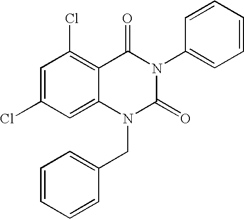 |
| III-9 | 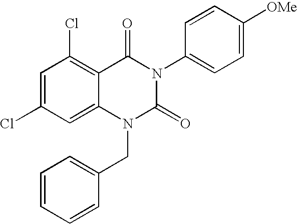 |
| III-10 | 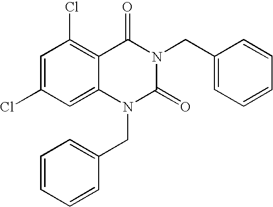 |
| III-11 | 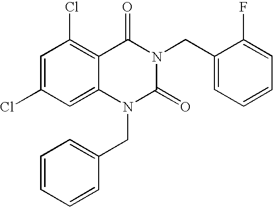 |
| III-12 | 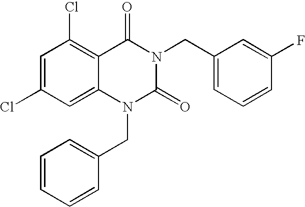 |
| III-13 | 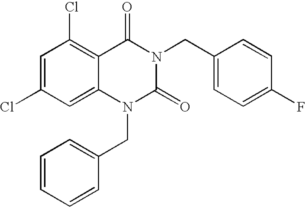 |
| III-14 | 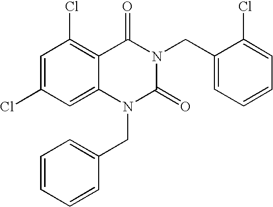 |
| III-15 | 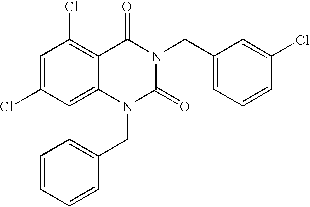 |
| III-16 | 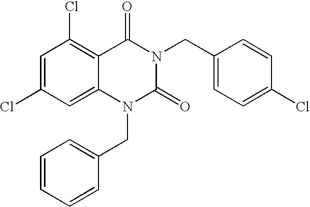 |
| III-17 | 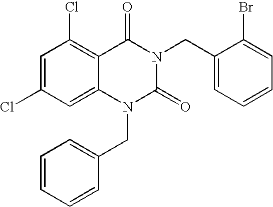 |
| III-18 | 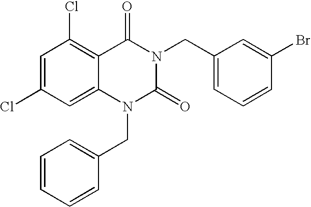 |
| III-19 | 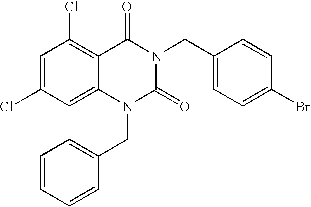 |
| III-20 | 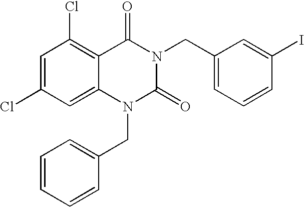 |
| III-21 | 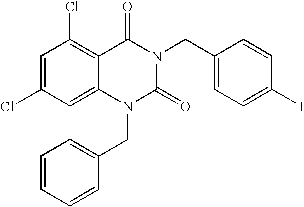 |
| III-22 | 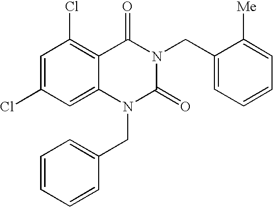 |
| III-23 | 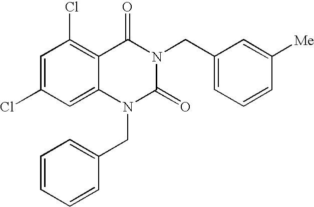 |
| III-24 | 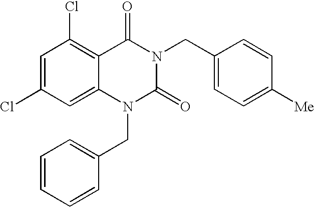 |
| III-25 | 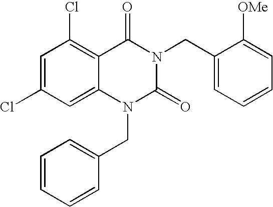 |
| III-26 | 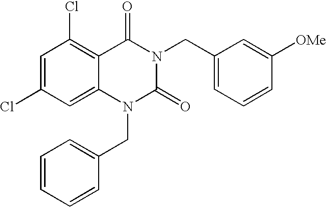 |
| III-27 | 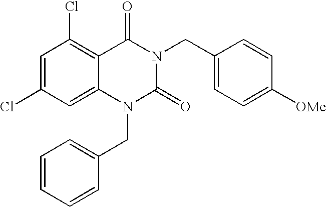 |
| III-28 | 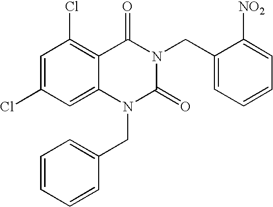 |
| III-29 | 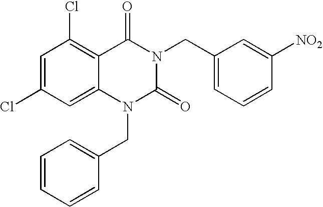 |
| III-30 | 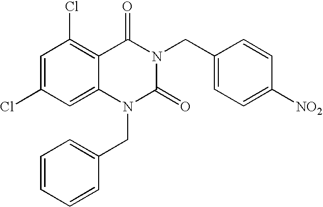 |
| III-31 | 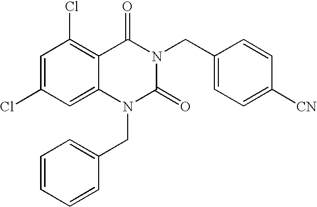 |
| III-32 | 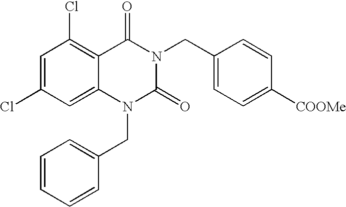 |
| III-33 | 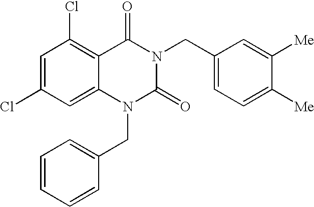 |
| III-34 | 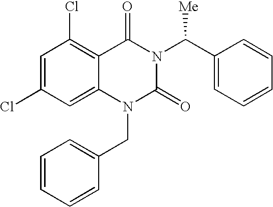 |
| III-35 | 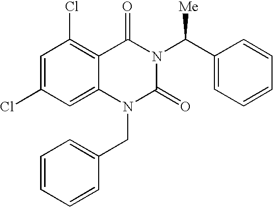 |
| III-36 | 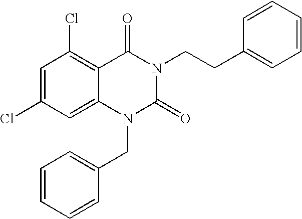 |
| III-37 | 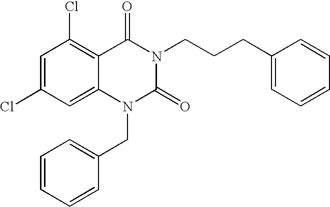 |
| III-38 | 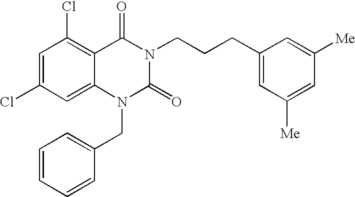 |
| III-39 | 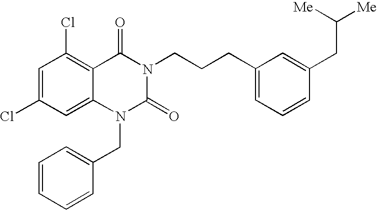 |
| III-40 | 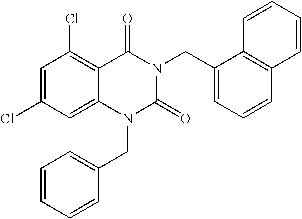 |
| III-41 | 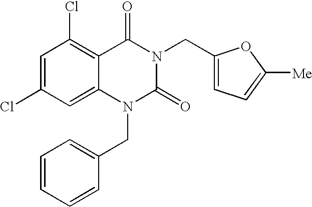 |
| III-42 | 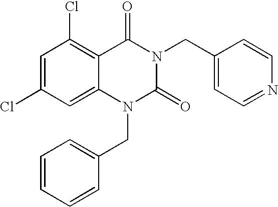 |
| III-43 | 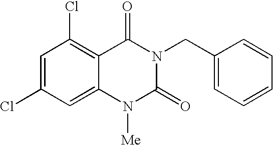 |
| III-44 | 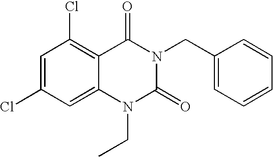 |
| III-45 | 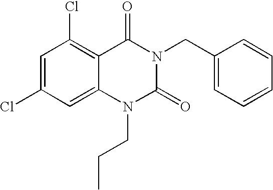 |
| III-46 | 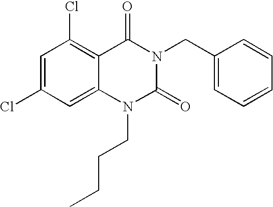 |
| III-47 | 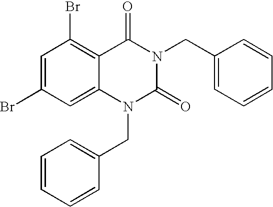 |
| III-48 | 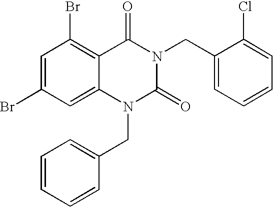 |

### Example

### Preparation of 1,3-Disubstituted 5-(4-methyl-piperazin-1-yl)-1H-quinazoline-2,4-dione

### Example 1

### Preparation of 1-benzyl-7-chloro-3-methyl-5-(4-methyl-piperazin-1-yl)-1H-quinazoline-2,4-dione

A mixture of Intermediate III-1 (1.0 mmol) and N-methylpiperazine (5 ml) was heated at flux temperature until the starting material was completely consumed. The reaction mixture was cooled to room temperature and the solvent was evaporated in a vacuum. The residue thus formed was dissolved in ethylacetate and washed with 0.5 M HQ, water and brine. The organic layer was dried over anhydrous magnesium sulfate and concentrated in a vacuum. Purification through flash column chromatography (eluent: a mixture of methylene chloride and methanol) gave the title compound as an ivory solid (83%). In the meantime, in contrast, a side product, in which the N-methylpiperidinyl group was present at position 7, was produced in a very small amount (<5%).

m.p. 173-175° C.;

¹H NMR (200 MHz, CDCl₃) δ 2.39 (s, 3H, NCH₃), 2.70-2.72 (m, 4H, NCH₂×2), 3.10-3.14 (m, 4H, NCH₂×2), 3.49 (d, 3H, J=1.2 Hz, NCH₃), 5.31 (s, 2H, NCH₂Ar), 6.69-6.72 (m, 2H, ArH), 7.21-7.35 (m, 5H, ArH);

MS (EI) m/e 398 [M⁺], 363, 306, 207, 91;

HRMS m/e Calcd. for C₂₁H₂₃N₄O₂Cl₁ 398.1509. Found 398.1523.

### Example 2

### Preparation of 1-benzyl-7-chloro-3-ethyl-5-(4-methyl-piperazin-1-yl)-1H-quinazoline-2,4-dione

The same procedure as in Example 1, with the exception that Intermediate III-2 was used, was repeated to afford the title compound as a pale yellow solid (83%).)

m.p. 155-159° C.;

¹H NMR (200 MHz, CDCl₃) δ 1.29 (t, 3H, J=6.9 Hz, CH₃), 2.39 (s, 3H, NCH₃), 2.67-2.70 (m, 4H, NCH₂×2), 3.12-3.16 (m, 4H, NCH₂×2), 4.11 (m, 2H, NCH₂), 5.31 (s, 2H, NCH₂Ar), 6.66-6.70 (m, 2H, ArH), 7.20-7.39 (m, 5H, ArH);

MS (EI) m/e 412 [M⁺], 223, 126;

HRMS m/e Calcd. for C₂₂H₂₅N₄O₂Cl₁ 412.1665 Found 412.1666.

### Example 3

### Preparation of 1-benzyl-7-chloro-5-(4-methyl-piperazin-1-yl)-3-propyl-1H-quinazoline-2,4-dione

The same procedure as in Example 1 with the exception that Intermediate III-3 was used, was repeated to afford the title compound as a pale yellow solid (45%).

m.p. 120-121° C.;

¹H NMR (200 MHz, CDCl₃) δ 0.94 (t, 3H, J=7.3 Hz, CH₃), 1.74 (m, 2H, CH₂), 2.39 (s, 3H, NCH₃), 2.69-2.71 (m, 4H, NCH₂×2), 3.13-3.31 (m, 4H, NCH₂×2), 4.02 (t, 2H, J=7.7 Hz, NCH₂), 5.30 (s, 2H, NCH₂Ar), 6.66 (d, 1H, J=2.0 Hz, ArH) 6.70 (d, 1H, J=2.0 Hz, ArH), 7.22-7.38 (m, 5H, ArH);

MS (EI) m/e 426 [M+], 391, 382, 327, 249, 126, 91;

HRMS m/e Calcd. for C₂₃H₂₇N₄O₂Cl₁ 426.1822. Found 426.1825.

### Example 4

### Preparation of 1-benzyl-3-butyl-7-chloro-5-(4-methyl-piperazin-1-yl)-1H-quinazoline-2,4-dione

The same procedure as in Example 1 with the exception that Intermediate III-4 was used, was repeated to afford the title compound as a pale yellow solid (70%).

m.p. 121-123° C.;

¹H NMR (200 MHz, CDCl₃) δ 0.92 (t, 3H, J=13 Hz, CH₃), 1.42 (m, 2H, CH₂), 1.68

(m, 2H, CH₂), 2.39 (s, 3H, NCH₃), 2.69-2.71 (m, 4H, NCH₂×2), 3.10-3.14 (m, 4H, NCH₂×2), 4.08 (t, 2H, J=7.3 Hz, NCH₂), 5.30 (s, 2H, NCH₂Ar), 6.65 (d, 1H, J=2.0 Hz, ArH) 6.69 (d, 1H, J=2.0 Hz, ArH), 7.20-7.38 (m, 5H, ArH);

MS (EI) m/e 440 [M⁺], 383, 340, 158, 91;

HRMS m/e Calcd. for C₂₄H₂₉N₄O₂Cl₁ 440.1979. found 440.1996.

### Example 5

### Preparation of 1-benzyl-7-chloro-3-(3-methyl-butyl)-5-(4-methyl-piperazin-1-yl)-1H-quinazoline-2,4-dione

The same procedure as in Example 1 with the exception that Intermediate III-5 was used, was repeated to afford the title compound as a pale yellow solid (55%).

m.p. 62-68° C.;

¹H NMR (200 MHz, CDCl₃) δ 0.97 (d, 6H, J=6.5 Hz, CH₃×2), 1.52-1.73 (m, 3H, CH₂&CH), 2.39 (s, 3H, NCH₃), 2.67-2.72 (m, 4H, NCH₂×2), 3.11-3.15 (m, 4H, NCH₂×2), 4.07 (t, 2H, J=13 Hz, NCH₂), 5.30 (s, 2H, NCH₂Ar), 6.65-6.69 (m, 2H, ArH), 7.20-7.38 (m, 5H, ArH);

MS (EI) m/e 454 [M⁺], 419, 384, 355, 327, 292, 193, 91, 70;

HRMS m/e Calcd. for C₂₅H₃₁N₄O₂Cl₁ 454.2135. Found 454.2141.

### Example 6

### Preparation of 1-benzyl-7-chloro-3-cyclohexylmethyl-5-(4-methyl-piperazin-1-yl)-1H-quinazoline-2,4-dione

The same procedure as in Example 1 with the exception that Intermediate III-6 was used, was repeated to afford the title compound as a pale yellow solid (40%).

m.p. 163-169° C.;

¹H NMR (200 MHz, CDCl₃) δ 1.16-1.26 (m, 5H, 2×CH₂&cyclohexyl [symbol] CH), 1.63 (s, 6H, 3×cyclohexyl [symbol] CH₂), 2.39 (s, 3H, NCH₃), 2.66-2.71 (m, 4H, NCH₂×2), 3.13 (m, 4H, NCH₂×2), 3.96 (d, 2H, J=7.4 Hz, NCH₂Ar), 5.29 (s, 2H, NCH₂Ar), 6.65 (s, 1H, ArH), 6.68 (s, 1H, ArH), 7.20-7.34 (m, 5H, ArH);

MS (EI) m/e 423[M⁺];

HRMS m/e Calcd. for C₂₇H₃₃N₄O₂Cl₁ 480.2292. Found 480.2274.

### Example 7

### Preparation of 1-benzyl-7-chloro-5-(4-methyl-piperazin-1-yl)-3-octyl-1H-quinazoline-2,4-dione

The same procedure as in Example 1 with the exception that Intermediate III-7 was used, was repeated to afford the title compound as a white solid (54%).

m.p. 78-80° C.;

¹H NMR (200 MHz, CDCl₃) δ 0.87 (m, 3H, CH₃), 1.27-1.33 (m, 10H, CH₂×5), 1.63 (m, 2H, CH₂), 2.39 (s, 3H, NCH₃), 2.67-2.70 (m, 4H, NCH₂×2), 3.12-3.15 (m, 4H, NCH₂×2), 4.08 (t, 2H, J=7.3 Hz, NCH₂), 5.30 (d, 2H, J=1.2 Hz, NCH₂Ar), 6.66-6.69 (m, 2H, ArH), 7.20-7.35 (m, 5H, ArH);

MS (EI) m/e 495 [M⁺], 461, 396, 207, 91;

HRMS m/e Calcd. for C₂₈H₃₇N₄O₂Cl₁ 496.2605. Found 496.2598.

### Example 8

### Preparation of 1-benzyl-7-chloro-5-(4-methyl-piperazin-1-yl)-3-phenyl-1H-quinazoline-2,4-dione

The same procedure as in Example 1 with the exception that Intermediate III-8 was used, was repeated to afford the title compound as a pale yellow solid (71%).

m.p. 111-119° C.;

¹H NMR (200 MHz, CDCl₃) δ 2.31 (s, 3H, NCH₃), 2.58-2.63 (m, 4H, NCH₂×2), 3.11
3.13 (m, 4H, NCH₂×2), 5.29 (d, 2H, J=6.2 Hz, NCH₂Ar), 6.74 (s, 1H, ArH), 6.76 (s, 1H, ArH), 7.27-7.55 (m, 10H, ArH);

MS (EI) m/e 460[M⁺];

HRMS m/e Calcd. for C₂₆H₂₅N₄O₂Cl₁ 460.1666. Found 460.1659.

### Example 9

### Preparation of 1-benzyl-7-chloro-3-(4-methoxy-phenyl)-5-(4-methyl-piperazin-1-yl)-1H-quinazoline-2,4-dione

The same procedure as in Example 1 with the exception that Intermediate III-9 was used, was repeated to afford the title compound as a yellow solid (76%).

m.p. 155-158° C.;

¹H NMR (200 MHz, CDCl₃) δ 2.14 (s, 3H, NCH₃), 2.33 (m, 4H, NCH₂×2), 3.36 (m, 4H, NCH₂×2), 3.83 (s, 3H, OCH₃), 4.83 (s, 2H, NCH₂Ar), 6.92 (d, 2H, J=9.2 Hz, ArH), 7.08 (d, 2H, J=1.8 Hz, ArH), 7.31-7.27 (m, 5H, ArH), 7.48 (d, 2H, J=9.2 Hz, ArH);

MS (EI) m/e 490[M⁺].

HRMS m/e Calcd. for C₂₇H₂₇Cl₁N₄O₃ 490.1772. Found 490.1769.

### Example 10

### Preparation of 1,3-dibenzyl-7-chloro-5-(4-methyl-piperazin-1yl)-1H-quinazoline-2,4-dione

The same procedure as in Example 1 with the exception that Intermediate III-10 was used, was repeated to afford the title compound as a pale yellow solid (60%).

m.p. 113-116° C.;

¹H NMR (200 MHz, CDCl₃) δ 2.38 (s, 3H, NCH₃), 2.70 (m, 4H, NCH₂×2), 3.12 (m, 4H, NCH₂×2), 5.28 (s, 2H, NCH₂Ar), 5.29 (s, 2H, NCH₂Ar), 6.65 (d, 1H, J=1.6 Hz, ArH), 6.68 (d, 1H, J=1.6 Hz, ArH), 7.18-7.36 (m, 8H, ArH), 7.52 (d, 2H, J=7.6 Hz, ArH);

MS (EI) m/e 475[M⁺];

HRMS m/e Calcd. for C₂₇H₂₇N₄O₂Cl₁ 474.1822. Found 474.1800.

### Example 11

### Preparation of 1-benzyl-7-chloro-3-(2-fluoro-benzyl)-5-(4-methyl-piperazin-1-yl)-1H-quinazoline-2,4-dione

The same procedure as in Example 1 with the exception that Intermediate III-11 was used, was repeated to afford the title compound as a pale yellow solid (61%).

m.p. 147-149° C.;

¹H NMR (200 MHz, CDCl₃) δ 2.37 (s, 3H, NCH₃), 2.63-2.68 (m, 4H, NCH₂×2), 3.12 (m, 4H, NCH₂×2), 5.29 (s, 2H, NCH₂Ar), 5.38 (s, 2H, NCH₂Ar), 6.67 (d, 1H, J=1.6 Hz, ArH), 6.69 (d, 1H, J=1.6 Hz, ArH), 7.03-7.34 (m, 9H, ArH);

MS (EI) m/e 492[M⁺];

HRMS m/e Calcd. for C₂₇H₂₆N₄O₂F₁Cl₁ 492.1728. Found 492.1708.

### Example 12

### Preparation of 1-benzyl-7-chloro-3-(3-fluoro-benzyl)-5-(4-methyl-piperazin-1-yl)-1H-quinazoline-2,4-dione

The same procedure as in Example 1 with the exception that Intermediate III-12 was used, was repeated to afford the title compound as a pale yellow solid (57%).

m.p. 148-150° C.;

¹H NMR (200 MHz, CDCl₃) δ 2.38 (s, 3H, NCH₃), 2.6-2.70 (m, 4H, NCH₂×2), 3.12 (m, 4H, NCH₂×2), 5.27 (s, 2H, NCH₂Ar), 5.29 (s, 2H, NCH₂Ar), 6.65 (d, 1H, J=1.8 Hz, ArH), 6.69 (d, 1H, J=1.8 Hz, ArH), 6.94 (m, 1H, ArH), 7.18-7.38 (m, 8H, ArH);

MS (EI) m/e 492[M⁺]; HRMS m/e Calcd. for C₂₇H₂₆N₄O₂F₁Cl₁ 492.1728. Found 492.1718.

### Example 13

### Preparation of 1-benzyl-7-chloro-3-(4-fluoro-benzyl)-5-(4-methyl-piperazin-1-yl)-1H-quinazoline-2,4-dione

The same procedure as in Example 1 with the exception that Intermediate III-13 was used, was repeated to afford the title compound as a pale yellow solid (67%).

m.p. 110-114° C.;

¹H NMR (200 MHz, CDCl₃) δ 2.39 (s, 3H, NCH₃), 2.66-2.70 (m, 4H, NCH₂×2), 3.12 (m, 4H, NCH₂×2), 5.24 (s, 2H, NCH₂Ar), 5.28 (s, 2H, NCH₂Ar), 6.64 (d, 1H, J=1.6 Hz, ArH), 6.68 (d, 1H, J=1.6 Hz, ArH), 6.94-7.02 (m, 2H, ArH), 7.17-7.33 (m, 5H, ArH), 7.47-7.54 (m, 2H, ArH);

MS (ET) m/e 492 [M⁺];

HRMS m/e Calcd. for C₂₇H₂₆N₄O₂F₁Cl₁ 492.1728. Found 492.1716.

### Example 14

### Preparation of 1-benzyl-3-(2-bromo-benzyl)-7-chloro-5-(4-methyl-piperazin-1-yl)-1H-quinazoline-2,4-dione

The same procedure as in Example 1 with the exception that Intermediate III-17 was used, was repeated to afford the title compound as a white solid (73%).

m.p. 91-93° C.;

¹H NMR (200 MHz, CDCl₃) δ 2.35 (s, 3H, NCH₃), 2.58-2.63 (m, 4H, NCH₂×2), 3.10-3.14 (m, 4H, NCH₂×2), 5.32 (s, 2H, NCH₂Ar), 5.38 (s, 2H, NCH₂Ar), 6.72 (s, 2H, ArH), 6.96 (d, 1H, J=7.6 Hz, ArH), 7.05-7.39 (m, 7H, ArH), 7.59 (d, 1H, J=7.6 Hz, ArH);

MS (EI) m/e 553 [M⁺];

HRMS m/e Calcd. for C₂₇H₂₆N₄O₂Cl₁Br₁ 552.0927. Found 552.0919.

### Example 15

### Preparation of 1-benzyl-3-(3-bromo-benzyl)-7-chloro-5-(4-methyl-piperazin-1-yl)-1H-quinazoline-2,4-dione

The same procedure as in Example 1 with the exception that Intermediate III-18 was > used, was repeated to afford the title compound as a yellow solid (75%).

m.p. 154-157° C.;

¹H NMR (200 MHz, CDCl₃) δ 2.39 (m, 3H, NCH₃), 2.68-2.72 (m, 4H, NCH₂×2), 3.10-3.14 (m, 4H, NCH₂×2), 5.26 (s, 2H, NCH₂Ar), 5.30 (s, 2H, NCH₂Ar), 6.70 (d, 2H, J=6.8 Hz, ArH), 7.18-7.40 (m, 8H, ArH), 7.64 (s, 1H, ArH);

MS (EI) m/e 553 [M⁺] 468, 340;

HRMS m/e Calcd. for C₂₇H₂₆N₄O₂Cl₁Br₁552.0927. Found 552.0928.

### Example 16

### Preparation of 1-benzyl-3-(4-bromo-benzyl)-7-chloro-5-(4-methyl-piperazin-1-yl)-1H-quinazoline-2,4-dione

The same procedure as in Example 1 with the exception that Intermediate III-19 was used, was repeated to afford the title compound as a white solid (69%).

m.p. 93-98° C.;

¹H NMR (200 MHz, CDCl₃) δ 2.39 (s, 3H, NCH₃), 2.63-2.67 (m, 4H, NCH₂×2), 3.06-3.10 (m, 4H, NCH₂×2), 5.23 (s, 2H, NCH₂Ar), 5.28 (s, 2H, NCH₂Ar), 6.65 (d, 1H, J=1.8 Hz, ArH), 6.79 (d, 1H, J=1.8 Hz, ArH), 7.17-7.46 (m, 9H, ArH);

MS (EI) m/e 553 [M⁺];

HRMS m/e Calcd. for C₂₇H₂₆N₄O₂Cl₁Br₁ 552.0927. Found 552.0909.

### Example 17

### Preparation of 1-benzyl-7-chloro-3-(2-chloro-benzyl)-5-(4-methyl-piperazin-1-yl)-1H-quinazoline-2,4-dione

The same procedure as in Example 1 with the exception that Intermediate III-14 was used, was repeated to afford the title compound as a white solid (73%).

m.p. 87-92° C.;

¹H NMR (200 MHz, CDCl₃) δ 2.35 (s, 3H, NCH₃), 2.60-2.63 (m, 4H, NCH₂×2), 3.06-3.09 (m, 4H, NCH₂×2), 5.32 (s, 2H, NCH₂Ar), 5.42 (s, 2H, NCH₂Ar), 6.72 (s, 2H, ArH), 7.03 (d, 2H, J=5.0 Hz, ArH), 7.14-7.40 (m, 7H, ArH);

MS (EI) m/e 508 [M⁺];

HRMS m/e Calcd. for C₂₇H₂₆N₄O₂Cl₂ 508.1432. Found 508.1433.

### Example 18

### Preparation of 1-benzyl-7-chloro-3-(3-chloro-benzyl)-5-(4-methyl-piperazin-1-yl)-1H-quinazoline-2,4-dione

The same procedure as in Example 1 with the exception that Intermediate III-15 was used, was repeated to afford the title compound as a white solid (64%).

m.p. 165-169° C.;

¹H NMR (200 MHz, CDCl₃) δ 2.39 (s, 3H, NCH₃), 2.64-2.67 (m, 4H, NCH₂×2), 3.07-3.09 (m, 4H, NCH₂×2), 5.26 (s, 2H, NCH₂Ar), 5.30 (s, 2H, NCH₂Ar), 6.66 (d, 1H, J=7.6 Hz, ArH), 6.70 (d, 1H, J=7.6 Hz, ArH), 7.18-7.39 (m, 8H, ArH);

MS (EI) m/e 508 [M⁺];

HRMS m/e Calcd. for C₂₇H₂₆N₄O₂Cl₂ 508.1432. Found 508.1452.

### Example 19

### Preparation of 1-benzyl-7-chloro-3-(4-chloro-benzyl)-5-(4-methyl-piperazin-1-yl)-1H-quinazoline-2,4-dione

The same procedure as in Example 1 with the exception that Intermediate III-16 was used, was repeated to afford the title compound as a white solid (69%).

m.p. 92-97° C.;

¹H NMR (200 MHz, CDCl₃) δ 2.39 (s, 3H, NCH₃), 2.43-2.45 (m, 4H, NCH₂×2), 2.80-2.84 (m, 4H, NCH₂×2), 5.24 (s, 2H, NCH₂Ar), 5.28 (s, 2H, NCH₂Ar), 6.65 (d, 1H, J=1.8 Hz, ArH), 6.69 (d, 1H, J=1.8 Hz, ArH), 7.20-7.34 (m, 7H, ArH), 7.47 (d, 2H, J=8.6 Hz, ArH);

MS (EI) m/e 509[M⁺];

HRMS m/e Calcd. for C₂₇H₂₆N₄O₂Cl₂ 508.1432. Found 508.1421.

### Example 20

### Preparation of 1-benzyl-7-chloro-3-(3-iodo-benzyl)-5-(4-methyl-piperazin-1-yl)-1H-quinazoline-2,4-dione

The same procedure as in Example 1 with the exception that Intermediate III-20 was used, was repeated to afford the title compound as a pale yellow solid (62%).

m.p. 182-184° C.;

¹H NMR (200 MHz, CDCl₃) δ 2.39 (s, 3H, NCH₃), 2.68-2.70 (m, 4H, NCH₂×2), 3.12 (m, 4H, NCH₂×2), 5.23 (s, 2H, NCH₂Ar), 5.29 (s, 2H, NCH₂Ar), 6.65 (d, 1H, J=2.0 Hz, ArH), 6.69 (d, 1H, J=2.0 Hz, ArH), 7.00 (t, 1H, J=7.8 Hz, ArH), 7.19-7.35 (m, 5H, ArH), 7.43 (d, 1H, J=8.2 Hz, ArH), 7.57 (d, 1H, J=8.2 Hz, ArH), 7.84 (s, 1H, ArH);

MS (EI) m/e 600 [M⁺];

HRMS m/e Calcd. for C₂₇H₂₆N₄O₂F₁Cl₁ 600.0789. Found 600.0792.

### Example 21

### Preparation of 1-benzyl-7-chloro-3-(4-iodo-benzyl)-5-(4-methyl-piperazin-1-yl)-1H-quinazoline-2,4-dione

The same procedure as in Example 1 with the exception that Intermediate III-21 was used, was repeated to afford the title compound as a pale yellow solid (59%).

m.p 114-118° C.;

¹H NMR (200 MHz, CDCl₃) δ 2.39 (s, 3H, NCH₃), 2.67-2.69 (m, 4H, NCH₂×2), 3.11 (m, 4H, NCH₂×2), 5.21 (s, 2H, NCH₂Ar), 5.28 (s, 2H, NCH₂Ar), 6.65 (s, 1H, ArH), 6.68 (s, 1H, ArH), 7.17-7.33 (m, 7H, ArH), 7.61 (d, 2H, J=8.2 Hz, ArH);

MD (EI) m/e 600 [M⁺];

HRMS m/e Calcd. for C₂₇H₂₆N₄O₂Cl₁I₁ 600.0789. Found 600.0784.

### Example 22

### Preparation of 1-benzyl-7-chloro-3-(2-methyl-benzyl)-5-(4-methyl-piperazin-1-yl)-1H-quinazoline-2,4-dione

The same procedure as in Example 1 with the exception that intermediate III-22 was used, was repeated to afford the title compound as a white solid (84%).

m.p. 92-95° C.;

¹H NMR (200 MHz, CDCl₃) δ 2.35 (s, 3H, CH₃), 2.46 (s, 3H, NCH₃), 2.60-2.64 (m, 4H, NCH₂×2), 3.07-3.09 (m, 4H, NCH₂×2), 5.28 (s, 2H, NCH₂Ar), 5.30 (s, 2H, NCH₂Ar), 6.70 (s, 2H, ArH), 6.98 (d, 1H, J=7.6 Hz, ArH), 7.06-7.25 (m, 7H, ArH), 7.34 (d, 1H, J=1.6 Hz, ArH);

MS (EI) m/e 488 [M⁺];

HRMS m/e Calcd. for C₂₈H₂₉N₄O₂Cl_{1488.1979}. Found 488.1981.

### Example 23

### Preparation of 1-benzyl-7-chloro-3-(3-methyl-benzyl)-5-(4-methyl-piperazin-1-yl)-1H-quinazoline-2,4-dione

The same procedure as in Example 1 with the exception that Intermediate III-23 was used, was repeated to afford the title compound as a white solid (89%).)

m.p. 171-175° C.;

¹H NMR (200 MHz, CDCl₃) δ 2.32 (s, 3H, CH₃), 2.39 (s, 3H, NCH₃), 2.64-2.67 (m, 4H, NCH₂×2), 3.07-3.09 (m, 4H, NCH₂×2), 5.27 (s, 2H, NCH₂Ar), 5.29 (s, 2H, NCH₂Ar), 6.65 (d, 1H, J=1.2 Hz, ArH), 6.68 (d, 1H, J=1.2 Hz, ArH), 7.08 (d, 1H, J=7.0 Hz, ArH), 7.20 (d, 1H, J=1.2 Hz, ArH), 7.26 (d, 1H, J=1.2 Hz, ArH), 7.23-7.36 (m, 6H, ArH);

MS (EI) m/e 488 [M⁺];

HRMS m/e Calcd. for C₂₈H₂₉N₄O₂Cl₁ 488.1979. Found 488.1977.

### Example 24

### Preparation of 1-benzyl-7-chloro-3-(4-methyl-benzyl)-5-(4-methyl-piperazin-1-yl)-1H-quinazoline-2,4-dione

The same procedure as in Example 1 with the exception that Intermediate III-24 was used, was repeated to afford the title compound as a white solid (92%).

m.p. 92-97° C.;

¹H NMR (200 MHz, CDCl₃) δ 2.31 (s, 3H, CH₃), 2.39 (s, 3H, NCH₃), 2.60-2.63 (m, 4H, NCH₂×2), 2.84-2.86 (m, 4H, NCH₂×2), 5.25 (s, 2H, NCH₂Ar), 5.28 (s, 2H, NCH₂Ar), 6.64 (d, 1H, J=1.6 Hz, ArH), 6.68 (d, 1H, J=1.6 Hz, ArH), 7.13 (m, 2H, ArH), 7.22 (m, 2H, ArH), 7.26 (m, 1H, ArH), 7.33 (d, 2H, J=7.0 Hz, ArH), 7.42 (d, 2H, J=7.8 Hz, ArH);

MS (EI) m/e 488[M⁺];

HRMS m/e Calcd. for C₂₈H₂₉N₄O₂Cl₁ 488.1979. Found 488.1987.

### Example 25

### Preparation of 1-benzyl-7-chloro-3-(2-methoxy-benzyl)-5-(4-methyl-piperazin-1-yl)-1H-quinazoline-2,4-dione

The same procedure as in Example 1 with the exception that Intermediate III-25 was used, was repeated to afford the title compound as a yellow solid (72%).

m.p. 91-96° C.;

¹H NMR (200 MHz, CDCl₃) δ 2.35 (m, 3H, NCH₃), 2.66 (m, 4H, NCH₂×2), 3.11 (m, 4H, NCH₂×2), 3.85 (s, 3H, OCH₃), 5.31 (d, 2H, J=2.4 Hz, NCH₂Ar), 5.34 (d, 2H, J=2.4 Hz, NCH₂Ar), 6.70 (s, 2H, ArH) 6.81-6.99 (m, 3H, ArH), 7.16-7.37 (m, 6H, ArH);

MS (EI) m/e 504 [M^{π}];

HRMS m/e Calcd. for C₂₈H₂₉N₄O₃Cl₁ 504.1928. Found 504.1930.

### Example 26

### Preparation of 1-benzyl-7-chloro-3-(3-methoxy-benzyl)-5-(4-methyl-piperazin-1yl)-1H-quinazoline-2,4-dione

The same procedure as in Example 1 with the exception that Intermediate III-26 was D used, was repeated to afford the title compound as a yellow solid (70%).

m.p. 98-110° C.;

¹H NMR (200 MHz, CDCl₃) δ 2.38 (s, 3H, NCH₃), 2.70 (m, 4H, NCH₂×2), 3.12 (m, 4H, NCH₂×2), 3.78 (s, 3H, OCH₃), 5.28 (s, 2H, NCH₂Ar), 5.29 (s, 2H, NCH₂Ar), 6.65 (d, 1H, J=1.6 Hz, ArH), 6.69 (d, 1H, J=1.6 Hz, ArH), 6.82 (d, 1H, J=7.4 Hz, ArH), 7.08 (d, 2H, J=6.6 Hz, ArH), 7.19-7.33 (m, 6H, ArH);

HRMS m/e Calcd. for C₂₈H₂₉N₄O₃Cl₁ 504.1928. Found 504.1922.

### Example 27

### Preparation of 1-benzyl-7-chloro-3-(4-methoxy-benzyl)-5-(4-methyl-piperazin-1-yl)-1H-quinazoline-2,4-dione

The same procedure as in Example 1 with the exception that Intermediate III-27 was used, was repeated to afford the title compound as a yellow solid (72%).

m.p. 81-89° C.;

¹H NMR (200 MHz, CDCl₃) δ 2.40 (m, 3H, NCH₃), 2.69-2.72 (m, 4H, NCH₂×2), 3.10-3.14 (m, 4H, NCH₂×2), 3.78 (s, 3H, OCH₃), 5.23 (s, 2H, NCH₂Ar), 5.29 (s, 2H, NCH₂Ar), 6.64 (d, 1H, J=1.6 Hz, ArH), 6.68 (d, 1H, J=1.6 Hz, ArH), 6.86 (d, 2H, J=8.8 Hz, ArH), 7.17-7.34 (m, 5H, ArH), 7.51 (d, 2H, J=8.8 Hz, ArH);

MS (EI) m/e 504[M⁺], 207;

HRMS m/e Calcd. for C₂₈H₂₉N₄O₃Cl₁ 504.1928. Found 504.1940.

### Example 28

### Preparation of 1-benzyl-7-chloro-3-(2-hydroxy-benzyl)-5-(4-methyl-piperazin-1-yl)-1H-quinazoline-2,4-dione

To a solution of the compound of Example 25 (0.10 g, 0.19 mmol) in methylene chloride (25 ml) was added boron tribromide (1 M in methylene chloride, 0.59 ml) at −78° C. The reaction solution was heated to room temperature with stirring for 12 hours. The reaction was terminated with cold water (5 ml), followed by extraction with methylene chloride (100 ml×3). The organic layer thus formed was dried over anhydrous magnesium sulfate and evaporated in a vacuum. The resulting residue was purified through flash column chromatography (methylene chloride:methanol=20:1) to afford the title compound as a white solid (89 mg, 95%).

m.p. 130-132° C.;

¹H NMR (200 MHz, CDCl₃) δ 2.44 (s, 3H, NCH₃), 2.75 (m, 4H, NCH₂×2), 3.15 (m, 4H, NCH₂×2), 5.26 (s, 2H, NCH₂Ar), 5.32 (s, 2H, NCH₂Ar), 6.71 (d, 1H, J=8.2 Hz, ArH), 6.84-6.97 (m, 2H, ArH), 7.20-7.36 (m, 7H, ArH), 7.72 (d, 1H, J=8.2 Hz, ArH);

MS (EI) m/e 490 [M⁺];

HRMS m/e Calcd. for C₂₇H₂₇N₄O₃Cl₁ 490.1771. Found 490.1763.

### Example 29

### Preparation of 1-benzyl-7-chloro-3-(3-hydroxy-benzyl)-5-(4-methyl-piperazin-1-yl)-1H-quinazoline-2,4-dione

The same procedure as in Example 28 with the exception that the compound of Example 26 was used, was repeated to afford the title compound as a white solid (83%).

m.p. 234-236° C.;

¹H NMR (200 MHz, CDCl₃) δ 2.73 (m, 4H, NCH₂×2), 3.12 (m, 4H, NCH₂×2), 3.49 (s, 3H, NCH₃), 5.20 (s, 2H, NCH₂Ar), 5.28 (s, 2H, NCH₂Ar), 6.67 (d, 2H, J=8.6 Hz, ArH), 6.74 (d, 2H, J=8.6 Hz, ArH), 7.17-7.43 (m, 6H, ArH);

MS (EI) m/e 491 [M⁺];

HRMS m/e Calcd. for C₂₇H₂₇N₄O₃Cl₁ 490.1771. Found 490.1769.

### Example 30

### Preparation of 1-benzyl-7-chloro-3-(4-hydroxy-benzyl)-5-(4-methyl-piperazin-1-yl)-1H-quinazoline-2,4-dione

The same procedure as in Example 28 with the exception that the compound of Example 27 was used, was repeated to afford the title compound as a white solid (75%).

m.p. 138-140° C.;

¹H NMR (200 MHz, CDCl₃) δ 2.74 (m, 4H, NCH₂×2), 3.13 (m, 4H, NCH₂×2), 3.48 (s, 3H, NCH₃), 5.24 (s, 2H, NCH₂Ar), 5.29 (s, 2H, NCH₂Ar), 6.68-6.75 (m, 3H, ArH), 6.93 (m, 1H, ArH), 7.02 (m, 1H, ArH), 7.13-7.22 (m, 3H, ArH), 7.27-2.34 (m, 3H, ArH);

MS (EI) m/e 490 [M⁺];

HRMS m/e Calcd. for C₂₇H₂₇N₄O₃Cl₁ 490.1771. Found 490.1771.

### Example 31

### Preparation of 1-benzyl-7-chloro-5-(4-methyl-piperazin-1-yl)-3-(2-nitro-benzyl)-1H-quinazoline-2,4-dione

The same procedure as in Example 1 with the exception that Intermediate III-28 was used, was repeated to afford the title compound as a pale yellow solid (92%).

m.p. 164-169° C.;

¹H NMR (200 MHz, CDCl₃) δ 2.35 (s, 3H, NCH₃), 2.63-2.65 (m, 4H, NCH₂×2), 3.11 (m, 4H, NCH₂×2), 5.31 (s, 2H, NCH₂Ar), 5.69 (s, 2H, NCH₂Ar), 6.72 (m, 2H, ArH), 7.20-7.56 (m, 8H, ArH), 8.06 (d, 1H, J=8.0 Hz, ArH);

MS (EI) m/e 519 [M⁺];

HRMS m/e Calcd. for C₂₇H₂₆N₄O₄Cl₁ 519.1673. Found 519.1676.

### Example 32

### Preparation of 1-benzyl-7-chloro-5-(4-methyl-piperazin-1-yl)-3-(3-nitro-benzyl)-1H-quinazoline-2,4-dione

The same procedure as in Example 1 with the exception that Intermediate III-29 was used, was repeated to afford the title compound as a pale yellow solid (65%).

m.p. 200-205° C.;

¹H NMR (200 MHz, CDCl₃) δ 2.39 (s, 3H, NCH₃), 2.66-2.70 (m, 4H, NCH₂×2), 3.13 (m, 4H, NCH₂×2), 5.30 (s, 2H, NCH₂Ar), 5.37 (s, 2H, NCH₂Ar), 6.68 (m, 1H, ArH), 6.71 (m, 1H, ArH), 7.19-7.38 (m, 5H, ArH), 7.45 (dd, 1H, J=8.0, 8.2 Hz, ArH), 7.81 (d, 1H, J=8.2 Hz, ArH), 8.11 (d, 1H, J=8.2 Hz, ArH), 8.34 (s, 1H, ArH);

MS (EI) m/e 519 [M⁺];

HRMS m/e Calcd. for C₂₇H₂₆N₅O₄Cl₁ 519.1673. Found 519.1678.

### Example 33

### Preparation of 1-benzyl-7-chloro-5-(4-methyl-piperazin-1-yl)-3-(4-nitro-benzyl)-1H-quinazoline-2,4-dione

The same procedure as in Example 1 with the exception that Intermediate III-30 was used, was repeated to afford the title compound as a pale yellow solid (67%).)

m.p. 109-113° C.;

¹H NMR (200 MHz, CDCl₃) δ 2.38 (s, 3H, NCH₃), 2.64 (m, 4H, NCH₂×2), 3.08 (m, 4H, NCH₂×2), 5.29 (s, 2H, NCH₂Ar), 5.36 (s, 2H, NCH₂Ar), 6.68 (s, 1H, ArH), 6.72 (s, 1H, ArH), 7.17-7.34 (m, 5H, ArH), 7.65 (d, 2H, J=8.6 Hz, ArH), 8.19 (d, 2H, J=8.6 Hz, ArH);

MS (EI) m/e 519[M⁺];

HRMS m/e Calcd. for C₂₇H₂₆N₅O₄Cl₁ 519.1673. Found 519.1669.

### Example 34

### Preparation of 3-(2-amino-benzyl)-1-benzyl-7-chloro-5-(4-methyl-piperazin-1-yl)-1H-quinazoline-2,4-dione

The compound of Example 31 (0.10 g, 0.19 mmol) was subjected to hydrogenation in a solution of charcoal-supported palladium in ethanol (10 ml). After the starting material was completely consumed, determined by monitoring using TLC, the inhomogeneous mixture was filtered on a cellite pad, and the filtrate was concentrated. The residue was purified through flash column chromatography (CH₂Cl₂: MeOH=20:1) to afford the title compound as a lemon colored solid (0.06 g, 52%).

¹H NMR (200 MHz, CDCl₃) δ 2.40 (s, 3H, NCH₃), 2.67-2.71 (m, 4H, NCH₂×2), 3.11 (m, 4H, NCH₂×2), 4.68 (br s, 2H, NH₂), 5.24 (s, 2H, NCH₂Ar), 5.30 (s, 2H, NCH₂Ar), 6.62 (s, 1H, ArH), 6.66 (s, 1H, ArH), 6.69 (d, 1H, J=7.0 Hz, ArH), 7.04 (t, 1H, J=7.2 Hz, ArH), 7.19-7.38 (m, 6H, ArH), 7.59-7.63 (d, 1H, J=7.6 Hz, ArH);

MS (EI) m/e 489[M⁺];

HRMS m/e Calcd. for C₂₇H₂₈N₄O₃Cl₁ 489.1931. Found 489.1926.

### Example 35

### Preparation of 3-(3-amino-benzyl)-1-benzyl-7-chloro-5-(4-methyl-piperazin-1-yl)-1H-quinazoline-2,4-dione

The same procedure as in Example 34 with the exception that the compound of Example 32 was used, was repeated to afford the title compound as a white solid (37%).

m.p. 260-265° C.;

¹H NMR (200 MHz, CDCl₃) δ 2.39 (s, 3H, NCH₃), 2.67-2.71 (m, 4H, NCH₂×2), 3.13 (m, 4H, NCH₂×2), 3.63 (br s, 2H, NH₂), 5.20 (s, 2H, NCH₂Ar), 5.29 (s, 2H, NCH₂Ar), 6.55 (d, 1H, J=7.6 Hz, ArH), 6.64 (d, 1H, J=1.6 Hz, ArH), 6.68 (d, 1H, J=1.6 Hz, ArH), 6.80-6.89 (m, 2H, ArH), 7.04 (t, 1H, J=1.6 Hz, ArH), 7.19-7.38 (m, 5H, ArH);

MS (EI) m/e 489[M⁺];

HRMS m/e Calcd. for C₂₇H₂₈N₅O₂Cl₁ 489.1931. Found 489.1939.

### Example 36

### Preparation of 3-(4-amino-benzyl)-1-benzyl-7-chloro-5-(4-methyl-piperazin-1-yl)-1H-quinazoline-2,4-dione

The same procedure as in Example 34 with the exception that the compound of Example 33 was used, was repeated to afford the title compound as a white solid (38%).

m.p. 93-96° C.;

¹H NMR (200 MHz, CDCl₃) δ 2.42 (m, 3H, NCH₃), 2.74 (m, 4H, NCH₂×2), 3.11 (m, 4H, NCH₂×2), 3.51 (br s, 2H, NH₂), 5.20 (s, 2H, NCH₂Ar), 5.32 (s, 2H, NCH₂Ar), 6.54-6.74 (m, 3H, ArH), 7.17-7.40 (m, 8H, ArH);

HRMS m/e Calcd. for C₂₇H₂₈N₅O₂Cl₁ 489.1931. Found 489.19122.

### Example 37

### Preparation of 4-[1-benzyl-7-chloro-5-(4-methyl-piperazin-1-yl)-2,4-dioxo-1,4-dihydro-2H-quinazoline-3-ylmethyl]-benzonitrile

The same procedure as in Example 1 with the exception that Intermediate III-31 was used, was repeated to afford the title compound as a pale yellow solid (53%).)

m.p. 99-105° C.;

¹H NMR (200 MHz, CDCl₃) δ 2.38 (s, 3H, ArCH₃), 2.66-2.69 (m, 4H, NCH₂×2), 3.12 (m, 4H, NCH₂×2), 5.29-5.30 (m, 2H, NCH₂Ar), 5.31 (m, 2H, NCH₂Ar), 6.67 (d, 1H, J=1.6 Hz, ArH), 6.71 (d, 1H, J=1.6 Hz, ArH), 7.17-7.34 (m, 5H, ArH), 7.59 (m, 4H, ArH);

MS (EI) m/e 499[M⁺];

HRMS m/e Calcd. for C₂₈H₂₆N₅O₂Cl₁ 499.1783. Found 499.1775.

### Example 38

### Preparation of 4-[1-benzyl-7-chloro-5-(4-methyl-piperazin-1-yl)-2,4-dioxo-1,4-dihydro-2H-quinazoline-3-ylmethyl]-benzoic acid methyl ester

The same procedure as in Example 1 with the exception that Intermediate III-32 was used, was repeated to afford the title compound as a pale yellow solid (53%).

m.p. 90-94° C.;

¹H NMR (200 MHz, CDCl₃) δ 2.38 (s, 3H, NCH₃), 2.67 (m, 4H, NCH₂×2), 3.12 (m, 4H, NCH₂×2), 3.90 (s, 3H, OMe), 5.29 (s, 2H, NCH₂Ar), 5.33 (s, 2H, NCH₂Ar), 6.66-6.70 (m, 2H, ArH), 7.18-7.35 (m, 5H, ArH), 7.49 (d, 2H, J=8.6 Hz, ArH), 7.96 (d, 2H, J=8.6 Hz, ArH);

MS (EI) m/e 532 [M⁺];

HRMS m/e Calcd. for C₂₉H₂₉N₄O₄Cl₁ 532.1877. Found 532.1872.

### Example 39

### Preparation of 1-benzyl-7-chloro-3-(3,4-dimethyl-benzyl)-5-(4-methyl-piperazin-1-yl)-1H-quinazoline-2,4-dione

The same procedure as in Example 1 with the exception that Intermediate III-33 was used, was repeated to afford the title compound as a pale yellow solid (67%).

m.p. 96-99° C.;

1H NMR (200 MHz, CDCl₃) δ 2.22 (s, 6H, ArCH₃×2), 2.39 (s, 3H, NCH₃), 2.68-2.70 (m, 4H, NCH₂×2), 3.12 (m, 4H, NCH₂×2), 5.23 (s, 2H, NCH₂Ar), 5.28 (s, 2H, NCH₂Ar), 6.63 (d, 1H, J=2.0 Hz, ArH), 6.67 (d, 1H, J=2.0 Hz, ArH), 7.03-7.33 (m, 8H, ArH);

MS (EI) m/e 502[M⁺];

HRMS m/e Calcd. for C₂₉H₃₁N₄O₂Cl₁ 502.2135. Found 502.2129.

### Example 40

### Preparation of 1-benzyl-7-chloro-5-(4-methyl-piperazin-1yl)-3-[(R)-1-phenyl-ethyl]-1H-quinazoline-2,4-dione

The same procedure as in Example 1 with the exception that Intermediate III-34 was used, was repeated to afford the title compound as a yellow solid (78%).

m.p. 108-115° C.;

¹H NMR (200 MHz, CDCl₃) δ 1.96 (d, 3H, J=7.0 Hz, CH₃), 2.36 (s, 3H, NCH₃), 2.59 (m, 2H, NCH₂), 3.03 (m, 4H, NCH₂×2), 5.22 (m, 2H, NCH₂), 6.43 (q, 1H, J=7.0 Hz, CH), 6.60 (d, 1H, J=1.4 Hz, ArH), 6.65, (d, 1H, J=1.4 Hz, ArH), 7.17 (d, 2H, J=7.8 Hz, ArH), 7.34-7.22 (m, 6H, ArH), 7.45 (d, 2H, J=7.0 Hz, ArH);

MS (EI) m/e [M₊-CH₃] 474;

HRMS m/e Calcd. for C₂₈H₂₉N₄O₂Cl₁ 489.1979. Found 489.1972.

### Example 41

### Preparation of 1-benzyl-7-chloro-5-(4-methyl-piperazin-1yl)-3-[(S)-1-phenyl-ethyl]-1H-quinazoline-2,4-dione

The same procedure as in Example 1 with the exception that Intermediate III-35 was used, was repeated to afford the title compound as a yellow solid (86%).

m.p. 135-137° C.;

¹H NMR (200 MHz, CDCl₃) δ 1.96 (d, 3H, J=7.4 Hz, CH₃), 2.36 (s, 3H, NCH₃), 2.59 (m, 4H, NCH₂×2), 3.03 (m, 4H, NCH₂×2), 5.23 (s, 2H, NCH₂Ar), 6.43 (q, 1H, J=7.8 Hz, CH), 6.61 (d, 1H, J=1.8 Hz, ArH), 6.65 (d, 1H, J=1.8 Hz, ArH), 7.17 (d, 2H, J=7.6 Hz, ArH), 7.24-7.36 (m, 6H, ArH), 7.46 (d, 2H, J=6.8 Hz, ArH);

MS (EI) m/e 488[M⁺];

HRMS m/e Calcd. for C₂₈H₂₉N₄O₂Cl₁ 488.1979. Found 488.1992.

### Example 42

### Preparation of 1-benzyl-7-chloro-5-(4-methyl-piperazin-1-yl)-3-phenethyl-1H-quinazoline-2,4-dione

The same procedure as in Example 1 with the exception that Intermediate III-36 was used, was repeated to afford the title compound as a pale yellow solid (76%).

m.p. 136-138° C.;

¹H NMR (200 MHz, CDCl₃) δ 2.39 (s, 3H, NCH₃), 2.65-2.99 (m, 4H, NCH₂×2), 3.02-3.06 (m, 6H, CH₂&NCH₂×2), 4.32-4.40 (m, 2H, NCH₂), 6.64-6.67 (m, 2H, NCH₂Ar), 7.15-7.35 (m, 12H, ArH);

HRMS m/e Calcd. for C₂₈H₂₉N₄O₂Cl₁ 488.1994. Found 488.1994.

### Example 43

### Preparation of 1-benzyl-7-chloro-5-(4-methyl-piperazin-1-yl)-3-(3-phenyl-propyl)-1H-quinazoline-2,4-dione

The same procedure as in Example 1 with the exception that Intermediate III-37 was used, was repeated to afford the title compound as a pale yellow solid (85%).

m.p. 55-63° C.;

¹H NMR (200 MHz, CDCl₃) δ 2.06 (m, 2H, CH₂), 2.39 (s, 3H, NCH₃), 2.69-2.78 (m, 6H, NCH₂×2&CH₂Ar), 3.13 (m, 4H, NCH₂×2), 4.13 (t, 2H, J=13 Hz, NCH₂), 5.29 (s, 2H, NCH₂Ar), 6.65-7.34 (m, 12H, ArH);

HRMS m/e Calcd. for C₂₉H₃₁N₄O₂Cl₁ 502.2114. Found 502.2114.

### Example 44

### Preparation of 1-benzyl-7-chloro-3-[3-(3,5-dimethyl-phenyl)-propyl]-5-(4-methyl-piperazin-1-yl)-1H-quinazoline-2,4-dione

The same procedure as in Example 1 with the exception that Intermediate III-38 was used, was repeated to afford the title compound as a pale yellow solid (62%).

m.p. 67-73° C.;

¹H NMR (200 MHz, CDCl₃) δ 2.02 (m, 2H, CH₂), 2.26 (s, 6H, CH₃×2), 2.39 (s, 3H, NCH₃), 2.67-2.72 (m, 6H, CH₂Ar & NCH₂×2), 3.12-3.15 (m, 4H, NCH₂×2), 4.13 (t, 2H, J=7.3 Hz, NCH₂), 5.29 (s, 2H, NCH₂Ar), 6.64 (d, 1H, J=1.6 Hz, ArH), 6.69 (d, 1H, J=1.6 Hz, ArH), 6.81 (s, 1H, ArH), 6.84 (s, 2H, ArH), 7.19-7.34 (m, 5H, ArH);

MS (EI) m/e 530 [M⁺], 145, 99, 91;

HRMS m/e Calcd. for C₃₁H₃₅N₄O₂Cl₁ 530.2448. Found 530.2446.

### Example 45

### Preparation of 1-benzyl-7-chloro-3-[3-(3-isobutyl-phenyl)-propyl]-5-(4-methyl-piperazin-1-yl)-1H-quinazoline-2,4-dione

The same procedure as in Example 1 with the exception that Intermediate III-39 was used, was repeated to afford the title compound as a yellow solid (60%).

m.p. 56-62° C.;

¹H NMR (200 MHz, CDCl₃) δ 0.82 (d, 3H, J=6.8 Hz, CH₃×1.10-1.36 (m, 4H, CH& CH₃), 1.55 (m, 2H, CH₂), 2.04 (m, 2H, CH₂), 2.39 (s, 3H, NCH₃), 2.68-2.70 (m, 6H, CH₂Ar & NCH₂×2), 3.11 (m, 4H, NCH₂×2), 3.89-4.03 (m, 2H, NCH₂), 5.24 (s, 2H, NCH₂Ar), 6.62 (d, 1H, J=1.8 Hz, ArH), 6.67 (d, 1H, J=1.8 Hz, ArH), 7.15-7.38 (m, 9H, ArH);

MS (EI) m/e 559 [M⁺], 368, 91;

HRMS m/e Calcd. for C₃₃H₃₉N₄O₂Cl₁ 558.2761. Found 558.2763.

### Example 46

### Preparation of 1-benzyl-7-chloro-5-(4-methyl-piperazin-1-yl)-3-naphthalen-1-ylmethyl-1H-quinazoline-2,4-dione

The same procedure as in Example 1 with the exception that Intermediate III-40 was used, was repeated to afford the title compound as a pale yellow solid (46%).

m.p. 75-89° C.;

¹H NMR (200 MHz, CDCl₃) δ 2.34 (s, 3H, NCH₃), 2.62-2.64 (m, 4H, NCH₂×2), 3.07-3.08 (m, 4H, NCH₂×2), 5.33 (s, 2H, NCH₂Ar), 5.82 (s, 2H, NCH₂Ar), 6.69 (m, 2H, ArH), 7.22-7.56 (m, 9H, ArH), 7.73 (d, 1H, J=8.5 Hz, ArH), 7.85 (d, 1H, J=6.9 Hz, ArH), 8.26 (d, 1H, J=7.7 Hz, ArH);

MS (EI) m/e 524 [M⁺], 384, 292, 250, 141, 91;

HRMS m/e Calcd. for C₃₁H₂₉N₄O₂Cl₁ 524.1979. Found 524.1971.

### Example 47

### Preparation of 1-benzyl-7-chloro-3-(5-methyl-furan-2-ylmethyl)-5-(4-methyl-piperazin-1-yl)-1H-quinazoline-2,4-dione

The same procedure as in Example 1 with the exception that Intermediate 1H-41 was used, was repeated to afford the title compound as a pale yellow solid (26%).

m.p. 132-136° C.;

¹H NMR (200 MHz, CDCl₃) δ 2.24 (s, 3H, ArCH₃), 2.39 (s, 3H, NCH₃), 2.66-2.71 (m, 4H, NCH₂×2), 3.13-3.31 (m, 4H, NCH₂×2), 5.25 (s, 2H, NCH₂Ar), 5.30 (s, 2H, NCH₂Ar), 5.88 (m, 1H, ArH), 6.25 (m, 1H, ArH), 6.64 (d, 1H, J=2.0 Hz, ArH), 6.67 (d, 1H, J=2.0 Hz, ArH), 7.21-7.34 (m, 5H, ArH);

MS (EI) m/e 478 [M⁺], 384, 95;

HRMS m/e Calcd. for C₂₆H₂₇N₄O₃Cl₁ 478.1771. Found 478.1760.

### Example 48

### Preparation of 1-benzyl-7-chloro-5-(4-methyl-piperazin-1-yl)-3-pyridin-4-ylmethyl-1H-quinazoline-2,4-dione

The same procedure as in Example 1 with the exception that Intermediate III-42 was used, was repeated to afford the title compound as a yellow solid (44%).

m.p. 94-100° C.;

¹H NMR (200 MHz, CDCl₃) δ 2.38 (s, 3H, NCH₃), 2.64-2.69 (m, 4H, NCH₂×2), 3.12 (m, 4H, NCH₂×2), 5.28 (s, 2H, NCH₂Ar), 5.29 (s, 2H, NCH₂Ar), 6.68 (d, 1H, J=2.0 Hz, ArH), 6.71 (d, 1H, J=2.0 Hz, ArH), 7.18-7.35 (m, 7H, ArH), 8.53-8.56 (m, 2H, ArH);

MS (EI) m/e 475 [M⁺];

HRMS m/e Calcd. for C₂₆H₂₆N₅O₂Cl₁ 475.1775. Found 475.1777.

### Example 49

### Preparation of 3-benzyl-7-chloro-5-(4-methyl-piperazin-1-yl)-1H-quinazoline-2,4-dione

The same procedure as in Example 1 with the exception that Intermediate III-10 was used, was repeated to afford the title compound as a white solid (90%).

m.p. 208-210° C.;

¹H NMR (200 MHz, CDCl₃) δ 2.39 (s, 3H, NCH₃), 2.67-2.71 (m, 4H, NCH₂×2), 3.12 (m, 4H, NCH₂×2), 5.21 (s, 2H, NCH₂Ar), 6.77 (d, 1H, J=2.0 Hz, ArH), 6.66 (d, 1H, J=2.0 Hz, ArH), 7.27-7.50 (m, 5H, ArH), 9.97 (br s, 1H, NH);

MS (EI) m/e 384 [M⁺], 284, 250, 91, 77;

HRMS m/e Calcd. for C₂₀H₂₁N₄O₂Cl₁ 384.1353. Found 384.1360.

### Example 50

### Preparation of 3-benzyl-7-chloro-1-methyl-5-(4-methyl-piperazin-1-yl)-1H-quinazoline-2,4-dione

The same procedure as in Example 1 with the exception that Intermediate III-43 was used, was repeated to afford the title compound as a pale yellow solid (62%).

m.p. 132-136° C.;

¹H NMR (200 MHz, CDCl₃) δ 2.39 (s, 3H, NCH₃), 2.68 (m, 4H, NCH₂×2), 3.13 (m, 4H, NCH₂×2), 3.52 (s, 3H, NCH₃), 5.23 (s, 2H, NCH₂Ar), 6.72 (d, 1H, J=2.0 Hz, ArH), 6.74 (d, 1H, J=2.0 Hz, ArH), 7.24-7.49 (m, 5H, ArH);

MS (EI) m/e 398 [M⁺], 362, 91;

HRMS m/e Calcd. for C₂₁H₂₃N₄O₂Cl₁ 398.1509. Found 398.1505.

### Example 51

### Preparation of 3-benzyl-7-chloro-1-ethyl-5-(4-methyl-piperazin-1-yl)-1H-quinazoline-2,4-dione

The same procedure as in Example 1 with the exception that Intermediate III-44 was used, was repeated to afford the title compound as a pale yellow solid (67%).

m.p. 68-75° C.;

¹H NMR (200 MHz, CDCl₃) δ 1.28 (t, 3H, J=7.3 Hz, NCH₂CH₃), 2.39 (s, 3H, NCH₃), 2.68-2.70 (m, 4H, NCH₂×2), 3.12 (m, 4H, NCH₂×2), 4.06 (q, 2H, J=6.9 Hz, NCH₂CH₃), 5.23 (s, 2H, NCH₂Ar), 6.72-6.74 (m, 2H, ArH), 7.29-7.49 (m, 5H, ArH);

MS (EI) m/e 412 [M⁺], 377, 321, 91, 77;

HRMS m/e Calcd. for C₂₂H₂₅N₄O₂Cl₁ 412.1666. Found 412.1657.

### Example 52

### Preparation of 3-benzyl-7-chloro-5-(4-methyl-piperazin-1-yl)-1-propyl-1H-quinazoline-2,4-dione

The same procedure as in Example 1 with the exception that intermediate III-45 was used, was repeated to afford the title compound as a yellow solid (91%).

¹H NMR (200 MHz, CDCl₃) δ 0.96 (t, 3H, J=7.4 Hz, NCH₂CH₂CH₃), 1.67-1.78 (m, 2H, NCH₂CH₂CH₃) 2.38 (s, 3H, NCH₃), 2.65-2.70 (m, 4H, NCH₂×2), 3.11 (m, 4H, NCH₂×2), 3.95 (t, 2H, J=7.6 Hz, NCH₂CH₂CH₃), 5.23 (s, 2H, NCH₂Ar), 6.69 (s, 1H, ArH), 6.71 (s, 1H, ArH), 7.22-7.47 (m, 5H, ArH);

MS (EI) m/e 426 [M⁺], 192, 91;

HRMS m/e Calcd. for C₂₃H₂₇N₄O₂Cl₁ 426.1822. Found 426.1818.

### Example 53

### Preparation of 3-benzyl-1-butyl-7-chloro-5-(4-methyl-piperazin-1-yl)-1H-quinazoline-2,4-dione

The same procedure as in Example 1 with the exception that Intermediate III-46 was used, was repeated to afford the title compound as a yellow solid (98%).

¹H NMR (200 MHz, CDCl₃) δ 0.94 (t, 3H, J=7.2 Hz, NCH₂CH₂CH₂CH₃), 1.37-1.48 (m, 2H, NCH₂CH₂CH₂CH₃), 1.64-1.72 (m, 2H, NCH₂CH₂CH₂CH₃), 2.38 (s, 3H, NCH₃), 2.65-2.70 (m, 4H, NCH₂×2), 3.11 (m, 4H, NCH₂×2), 3.99 (t, 2H, J=1.6 Hz, NCH₂CH₂CH₂CH₃), 5.23 (s, 2H, NCH₂Ar), 6.70 (s, 1H, ArH), 6.71 (s, 1H, ArH), 7.22-7.47 (m, 5H, ArH);

MS (EI) m/e 440 [M⁺], 383, 193, 91;

HRMS m/e Calcd. for C₂₄H₂₉N₄O₂Cl₁ 440.1979. Found 440.1983.

### Example 54

### Preparation of 1,3-dibenzyl-7-bromo-5-(4-methyl-piperazin-1-yl)-1H-quinazoline-2,4-dione

The same procedure as in Example 1 with the exception that Intermediate III-47 was used, was repeated to afford the title compound as a white solid (75%).

m.p. 80-84° C.;

¹H NMR (200 MHz, CDCl₃) δ 2.38 (s, 3H, NCH₃), 2.70 (m, 4H, NCH₂×2), 3.12 (m, 4H, NCH₂×2), 5.28 (s, 2H, NCH₂Ar), 5.29 (s, 2H, NCH₂Ar), 6.83 (d, 1H, J=3.6 Hz, ArH), 6.84 (d, 1H, J=3.6 Hz, ArH), 7.18-7.47 (m, 8H, ArH), 7.51 (d, 2H, J=8.2 Hz, ArH);

MS (EI) m/e 518 [M⁺];

HRMS m/e Calcd. For C₂₇H₂₆N₄O₂Br₁ 518.1317. Found 518.1290.

### Example 55

### Preparation of 1-benzyl-7-bromo-3-(2-chloro-benzyl)-5-(4-methyl-piperazin-1-yl)-1H-quinazoline-2,4-dione

The same procedure as in Example 1 with the exception that Intermediate III-48 was used, was repeated to afford the title compound as a white solid (94%).)

m.p. 104-108° C.;

¹H NMR (200 MHz, CDCl₃) δ 2.35 (s, 3H, NCH₃), 2.66 (m, 4H, NCH₂×2), 3.12 (m, 4H, NCH₂×2), 5.31 (s, 2H, NCH₂Ar), 5.41 (s, 2H, NCH₂Ar), 6.08 (s, 2H, ArH), 7.15-7.40 (m, 9H, ArH);

MS (EI) m/e 552 [M⁺];

HRMS m/e Calcd. for C₂₇H₂₆N₄O₂Cl₁Br₁ 552.0927. Found 552.0889.

### Example 56

### Preparation of 1-benzyl-7-chloro-3-(4-methyl-phenyl)-5-piperazin-1yl-1H-quinazoline-2,4-dione

To a solution of Intermediate III-9 (1.0 mmol) in MeCN (30 ml) were dissolved piperazine (3.0 mmol) and K₂CO₃ (3.0 mmol). The reaction mixture was heated with reflux. Following the complete consumption of Intermediate III-9, the solvent was evaporated in a vacuum. The residue thus formed was dissolved in ethylacetate and washed with 0.5 M HQ, water and brine. The organic layer was dried over anhydrous magnesium sulfate and concentrated in a vacuum. Purification through flash column chromatography (eluent; a mixture of methylene chloride and methanol) gave the title compound as a yellow solid (66%).

m.p. 120-124° C.;

¹H NMR (200 MHz, CDCl₃) δ 2.68 (m, 4H, NCH₂×2), 3.26 (m, 4H, NCH₂×2), 3.82 (s, 3H, OCH₃), 4.83 (s, 2H, NCH₂Ar), 6.91 (d, 2H, J=9.2 Hz, ArH), 7.10 (d, 2H, J=1.8 Hz, ArH), 7.28-7.31 (m, 5H, ArH), 7.44 (d, 2H, J=9.2 Hz, ArH);

MS (EI) m/e 476[M⁺];

HRMS m/e Calcd. for C₂₆H₂₅Cl₁ N₄O₃ 476.1615. Found 476.1618.

### Example 57

### Preparation of 1,3-dibenzyl-7-chloro-5-piperazin-1-yl-1H-quinazoline-2,4-dione

The same procedure as in Example 56 with the exception that Intermediate III-10 was used, was repeated to afford the title compound as a white solid (69%).

m.p. 115-123° C.;

¹H NMR (200 MHz, CDCl₃) δ 3.13 (m, 4H, NCH₂×2), 3.18 (m, 4H, NCH₂×2), 6.68 (s, 4H, NCH₂Ar×2), 7.19-7.34 (m, 8H, ArH), 7.47-7.60 (m, 2H, ArH);

MS (EI) m/e 460 [M⁺];

HRMS m/e Calcd. for C₂₆H₂₅N₄O₂Cl₁ 460.1666. Found 460.1670.

### Example 58

### Preparation of 1-benzyl-7-chloro-3-(2-fluoro-benzyl)-5-piperazin-1-yl-quinazoline-2,4-dione

The same procedure as in Example 56 with the exception that Intermediate III-11 was used, was repeated to afford the title compound as a pale yellow solid (54%).

m.p. 98-109° C.;

¹H NMR (200 MHz, CDCl₃) δ 3.08 (m, 8H, NCH₂×4), 5.30 (s, 2H, NCH₂Ar), 5.38 (s, 2H, NCH₂Ar), 6.67 (d, 1H, J=1.6 Hz, ArH), 6.69 (d, 1H, J=1.6 Hz, ArH), 6.98-7.38 (m, 9H, ArH);

MS (EI) m/e 478 [M⁺];

HRMS m/e Calcd. for C₂₆H₂₄N₄O₂F₁Cl₁ 478.1571. Found 478.1577.

### Example 59

### Preparation of 1-benzyl-7-chloro-3-(2-chloro-benzyl)-5-piperazin-1-yl-1H-quinazoline-2,4-dione

The same procedure as in Example 56 with the exception that Intermediate III-14 was used, was repeated to afford the title compound as a white solid (70%).)

m.p. 120-125° C.;

¹H NMR (200 MHz, CDCl₃) δ 3.07 (m, 8H, NCH₂×4), 5.32 (s, 2H, NCH₂Ar), 5.41 (s, 2H, NCH₂Ar), 6.72 (s, 2H, ArH), 6.98-7.03 (m, 1H, ArH), 7.14-7.27 (m, 5H, ArH), 7.35-7.40 (m, 3H, ArH);

MS (EI) m/e 494[M⁺];

HRMS m/e Calcd. for C₂₆H₂₄N₄O₂Cl₂ 494.1276. Found 494.1292.

### Example 60

### Preparation of 1-benzyl-3-(2-bromo-benzyl)-7-chloro-5-piperazin-1-yl-1H-quinazoline-2,4-dione

The same procedure as in Example 56 with the exception that Intermediate III-17 was used, was repeated to afford the title compound as a white solid (58%).

m.p. 94-97° C.;

¹H NMR (200 MHz, CDCl₃) δ 3.07 (m, 8H, NCH₂×4), 5.32 (s, 2H, NCH₂Ar), 5.38 (s, 2H, NCH₂Ar), 6.72 (s, 2H, ArH), 6.97 (d, 1H, J=7.8 Hz, ArH), 7.06-7.17 (m, 2H, ArH), 7.21-7.38 (m, 6H, ArH), 7.59 (d, 1H, J=7.8 Hz, ArH);

MS (EI) m/e 540[M⁺];

HRMS m/e Calcd. for C₂₆H₂₄N₄O₂Br₁Cl₁ 538.0771. Found 538.0773

### Example 61

### Preparation of 1-benzyl-5-chloro-3-(3-iodo-benzyl)-7-piperazin-1-yl-1H-quinazoline-2,4-dione

The same procedure as in Example 56 with the exception that Intermediate III-20 was used, was repeated to afford the title compound as a pale yellow solid (55%).

m.p. 98-104° C.;

¹H NMR (200 MHz, CDCl₃) δ 3.08-3.12 (m, 4H, NCH₂×2), 5.23 (s, 2H, NCH₂Ar), 5.29 (s, 2H, NCH₂Ar), 6.65 (d, 1H, J=1.8 Hz, ArH), 6.68 (d, 1H, J=1.8 Hz, ArH), 7.04 (t, 1H, J=7.8 Hz, ArH), 7.19-7.39 (m, 5H, ArH), 7.46 (m, 1H, ArH), 7.59 (m, 1H, ArH), 7.85 (s, 1H, ArH);

HRMS m/e Calcd. for C₂₆H₂₄N₄O₂Cl₁I₁ 586.0643. Found 586.0632.

### Example 62

### Preparation of 1-benzyl-5-chloro-3-(2-methoxy-benzyl)-7-piperazin-1-yl-1H-quinazoline-2,4-dione

The same procedure as in Example 56 with the exception that Intermediate III-22 was used, was repeated to afford the title compound as a white solid (66%).

m.p. 83-87° C.;

¹H NMR (200 MHz, CDCl₃) δ 2.47 (s, 3H, CH₃), 3.06 (m, 8H, NCH₂×4), 5.30 (s, 2H, NCH₂Ar), 5.32 (s, 2H, NCH₂Ar), 6.70 (s, 2H, ArH), 7.00-7.15 (m, 4H, ArH), 7.21-7.35 (m, 5H, ArH);

MS (EI) m/e 474[M⁺];

HRMS m/e Calcd. for C₂₇H₂₇N₄O₂Cl₁ 474.1822. Found 474.1826.

### Example 63

### Preparation of 1-benzyl-7-chloro-3-(2-methoxy-benzyl)-5-piperazin-1-yl-1H-quinazoline-2,4-dione

The same procedure as in Example 56 with the exception that Intermediate III-25 was used, was repeated to afford the title compound as a white solid (81%).

m.p. 122-125° C.;

¹H NMR (200 MHz, CDCl₃) δ 3.07 (m, 8H, NCH₂×4), 3.85 (s, 3H, OCH₃), 5.31 (s, 2H, NCH₂Ar), 5.34 (s, 2H, NCH₂Ar), 6.70 (s, 2H, ArH), 6.88 (d, 2H, J=8.0 Hz, ArH), 7.00 (s, 1H, ArH), 7.21 (s, 1H, ArH), 7.25-7.37 (m, 5H, ArH);

MS (EI) m/e 490[M⁺];

HRMS m/e Calcd. for C₂₇H₂₇N₄O₃Cl₁ 490.1771. Found 490.1781.

### Example 64

### Preparation of 1-benzyl-7-chloro-3-(3-methyoxy-benzyl)-5-piperazin-1-yl-1H-quinazoline-2,4-dione

The same procedure as in Example 56 with the exception that Intermediate III-26 was used, was repeated to afford the title compound as a yellow solid (73%).

m.p. 124-130° C.;

¹H NMR (200 MHz, CDCl₃) δ 3.12 (m, 8H, NCH₂×4), 3.78 (s, 3H, OCH₃), 5.28 (s, 2H, NCH₂Ar), 5.30 (s, 2H, NCH₂Ar), 6.67 (d, 2H, J=2.8 Hz, ArH), 6.82 (d, 1H, J=9.2 Hz, ArH), 7.07 (d, 2H, J=7.4 Hz, ArH), 7.19-7.37 (m, 6H, ArH);

MS (EI) m/e 440 [M⁺];

HRMS m/e Calcd. for C₂₇H₂₇N₄O₃Cl₁ 490.1772. Found 490.1779.

### Example 65

### Preparation of 1-benzyl-7-chloro-3-(2-hydroxy-benzyl)-5-piperazin-1-yl-1H-quinazoline-2,4-dione

To a solution of the compound of Example 63 (58 mg, 0.11 mmol) in methylene chloride (20 ml) was added boron tribromide (0.35 ml, 1M) at −78° C. The resulting solution was warmed to room temperature with stirring over a period of 12 hours. The reaction was terminated by cold water (5 ml), followed by extraction with methylene chloride (100 ml×3). The organic layer was dried over anhydrous magnesium sulfate and distilled in a vacuum. The residue was purified through flash column chromatography to afford the title compound as a white solid (40 mg, 77%).

m.p. 113-130° C.;

¹H NMR (200 MHz, CDCl₃) δ 3.39 (m, 4H, NCH₂×2), 3.51 (m, 4H, NCH₂×2), 5.25 (s, 2H, NCH₂Ar), 5.33 (s, 2H, NCH₂Ar), 6.78 (d, 2H, J=9.8 Hz, ArH), 6.88-6.96 (m, 2H, ArH), 7.20-7.36 (m, 6H, ArH), 7.65-7.69 (m, 1H, ArH);

MS (EI) m/e 476[M⁺];

HRMS m/e Calcd. for C₂₆H₂₅N₄O₃Cl₁476.1615. Found 476.1617.

### Example 66

### Preparation of 1-benzyl-7-chloro-3-(2-nitro-benzyl)-5-piperazin-1-yl-1H-quinazoline-2,4-dione

The same procedure as in Example 56 with the exception that Intermediate 1H-27 was used, was repeated to afford the title compound as a pale yellow solid (69%).

m.p. 129-134° C.;

¹H NMR (200 MHz, CDCl₃) δ 3.18 (m, 8H, NCH₂×4), 5.31 (s, 2H, NCH₂Ar), 5.67 (s, 2H, NCH₂Ar), 6.72 (s, 1H, ArH), 6.75 (s, 1H, ArH), 7.20-7.53 (m, 8H, ArH), 8.04 (d, 1H, J=8.2 Hz, ArH);

MS (EI) m/e 507[M⁺];

HRMS m/e Calcd. for C₂₆H₂₄N₅O₄Cl₁ 505.1516. Found 505.1518.

### Example 67

### Preparation of 3-(2-amino-benzyl)-1-benzyl-7-chloro-5-piperazin-1-yl-1H-quinazoline-2,4-dione

The compound of Example 66 (0.10 g, 0.20 mmol) was subjected to hydrogenation in a solution of charcoal-supported palladium in ethanol (10 ml). After the starting material was completely consumed, as monitored by TLC, the inhomogeneous mixture was filtered on a cellite pad, and the filtrate was concentrated. The residue was purified through flash column chromatography (CH₂Cl₂: MeOH=20:1) to afford the title compound as a lemon colored solid (0.03 g, 30%).

¹H NMR (200 MHz, CDCl₃) δ 3.08 (m, 4H, NCH₂×2), 3.14 (m, 4H, NCH₂×2), 5.24 (s, 2H, NCH₂Ar), 5.30 (s, 2H, NCH₂Ar), 6.63 (d, 1H, J=1.6 Hz, ArH), 6.66 (d, 1H, J=1.6 Hz, ArH), 6.69 (d, 1H, J=7.8 Hz, ArH), 7.04 (t, 1H, J=7.2 Hz, ArH), 7.19-7.34 (m, 6H, ArH), 7.59 (d, 1H, J=7=7.6 Hz, ArH);

MS (EI) m/e 475 [M⁺];

HRMS m/e Calcd. for C₂₆H₂₆N₅O₂Cl₁ 475.1775. Found 475.1773.

### Example 68

### Preparation of 1-benzyl-7-chloro-3-[(R)-1-phenyl-ethyl]-5-piperazin-1yl-1H-quinazoline-2,4-dione

The same procedure as in Example 56 with the exception that Intermediate III-34 was used, was repeated to afford the title compound as a yellow solid (65%).

m.p. 101-106° C.;

¹H NMR (200 MHz, CDCl₃) δ 1.96 (d, 3H, J=7.0 Hz, CH₃), 2.96 (m, 6H, NCH₂×3), 5.23 (s, 2H, NCH₂), 6.42 (q, 1H, J=7.4 Hz, NCH), 6.63 (d, 2H, J=5.2 Hz, ArH), 7.18 (d, 2H, J= 7.8 Hz, ArH), 7.22-7.43 (m, 6H, ArH), 7.46 (d, 2H, J=6.8 Hz, ArH);

HRMS m/e Calcd. for C₂₇H₂₇N₄O₂Cl₁ 474.1822. Found 474.1833.

### Example 69

### Preparation of 1-benzyl-7-chloro-3-[(S)-1-phenyl-ethyl]-5-piperazin-1yl-1H-quinazoline-2,4-dione

The same procedure as in Example 56 with the exception that Intermediate III-35 was used, was repeated to afford the title compound as a yellow solid (73%).

m.p. 91-96° C.;

¹H NMR (200 MHz, CDCl₃) δ 1.96 (d, 3H, J=7.0 Hz, CH₃), 3.00 (m, 8H, NCH₂×4), 5.23 (s, 2H, NCH₂Ar), 6.42 (q, 1H, J=7.4 Hz, NCH), 6.60 (d, 1H, J=1.8 Hz, ArH), 6.64 (d, 1H, J=1.8 Hz, ArH), 7.22 (d, 2H, J=9.0 Hz, ArH), 7.26-7.42 (m, 6H, ArH), 7.46 (d, 2H, J=7.2 Hz, ArH);

MS (EI) m/e 474 [M⁺];

HRMS m/e Calcd. for C₂₇H₂₇N₄O₂Cl₁ 474.1837. Found 474.1822.

### Example 70

### Preparation of 1-benzyl-7-chloro-3-phenethyl-5-piperazin-1-yl-1H-quinazoline-2,4-dione

The same procedure as in Example 56 with the exception that Intermediate III-36 was used, was repeated to afford the title compound as a pale yellow solid (71%).

m.p. 190-194° C.;

¹H NMR (200 MHz, CDCl₃) δ 2.61 (t, 2H, J=7.0 Hz, CH₂Ar), 2.99-3.12 (m, 9H, NCH₂×4&NH), 4.36 (m, 2H, NCH₂), 5.29 (s, 2H, NCH₂Ar), 6.65 (d, 2H, J=1.6 Hz, ArH), 7.16-7.35 (m, 10H, ArH);

HRMS m/e Calcd. for C₂₈H₂₉N₄O₂Cl₁ 488.1994. Found 488.1994.

### Example 71

### Preparation of 1-benzyl-7-chloro-3-(3-phenyl-propyl)-5-piperazin-1-yl-1H-quinazoline-2,4-dione

The same procedure as in Example 56 with the exception that Intermediate III-37 was used, was repeated to afford the title compound as a pale yellow solid (60%).

m.p. 62-68° C.;

¹H NMR (200 MHz, CDCl₃) δ 2.05 (m, 2H, CH₂), 2.23 (br s, 1H, NH), 2.69 (t, 2H, J=7.7 Hz, CH₂Ar), 3.09-3.13 (m, 8H, NCH₂×4), 4.12 (t, 2H, J=7.7 Hz, NCH₂), 5.29 (s, 2H, NCH₂Ar), 6.66-7.34 (m, 12H, ArH);

HRMS m/e Calcd. for C₂₈H₂₉N₄O₂Cl₁ 488.2019. Found 488.2019.

TABLE 2

| Example | Structure |
|---|---|
| 1 | 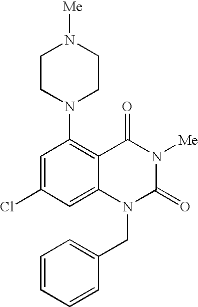 |
| 2 | 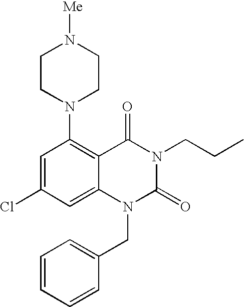 |
| 3 | 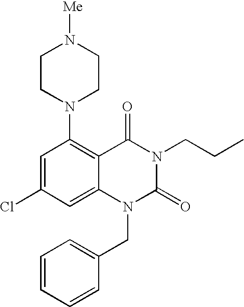 |
| 4 | 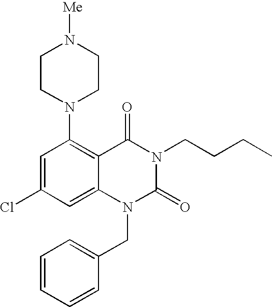 |
| 5 | 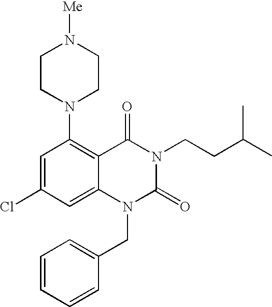 |
| 6 | 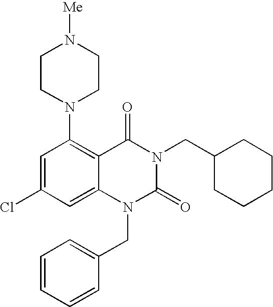 |
| 7 | 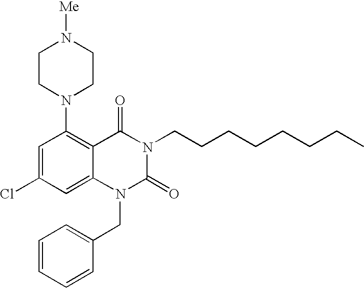 |
| 8 | 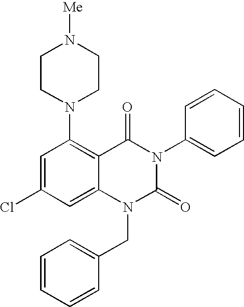 |
| 9 | 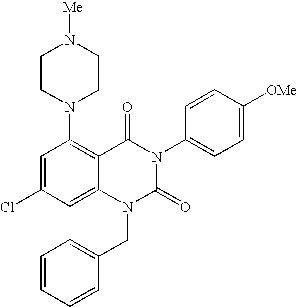 |
| 10 | 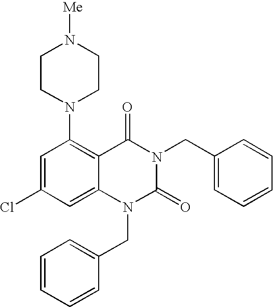 |
| 11 | 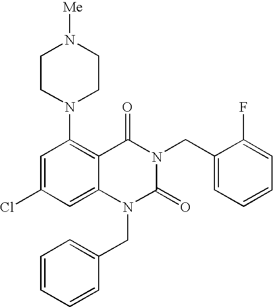 |
| 12 | 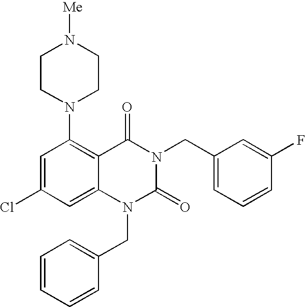 |
| 13 | 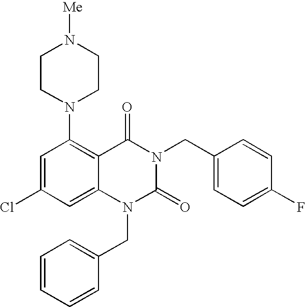 |
| 14 | 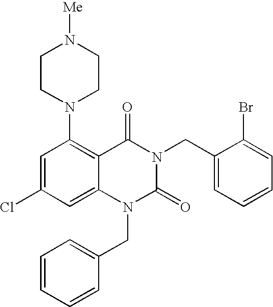 |
| 15 | 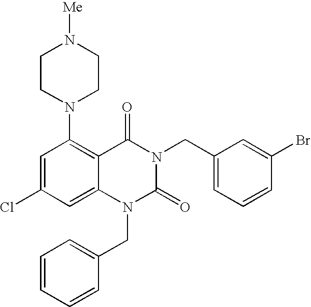 |
| 16 | 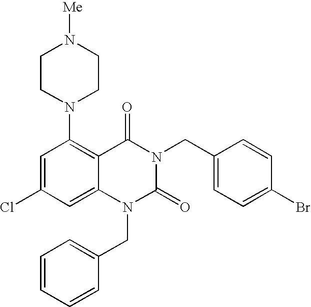 |
| 17 | 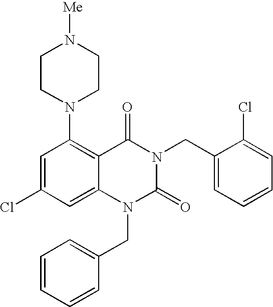 |
| 18 | 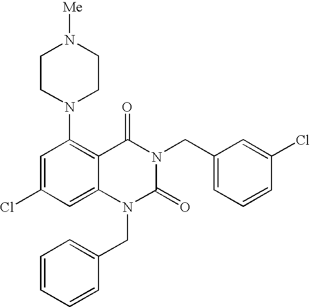 |
| 19 | 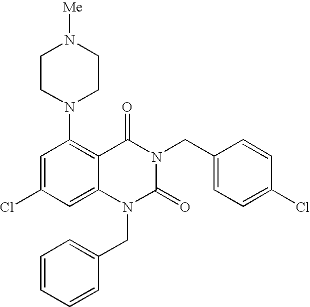 |
| 20 | 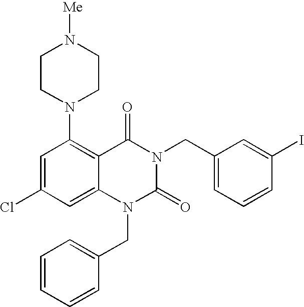 |
| 21 | 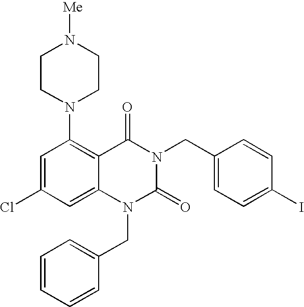 |
| 22 | 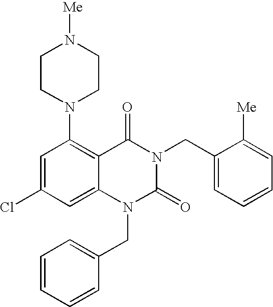 |
| 23 | 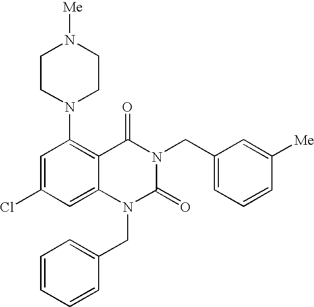 |
| 24 | 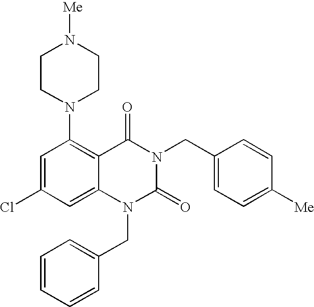 |
| 25 | 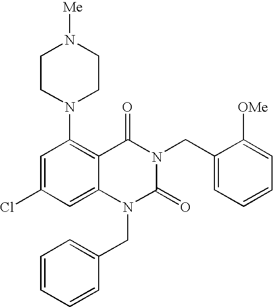 |
| 26 | 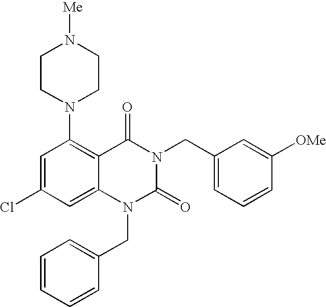 |
| 27 | 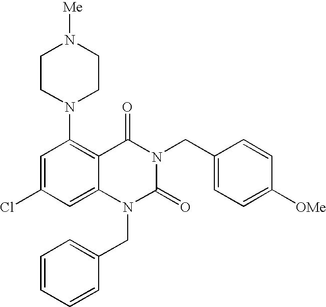 |
| 28 | 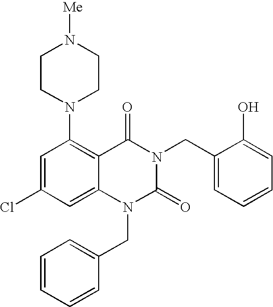 |
| 29 | 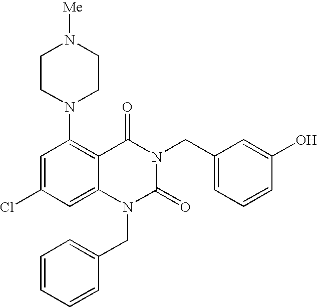 |
| 30 | 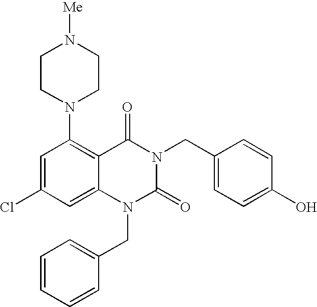 |
| 31 | 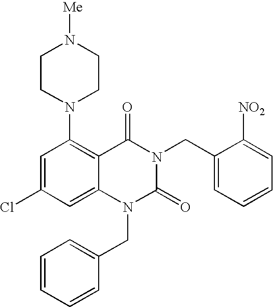 |
| 32 | 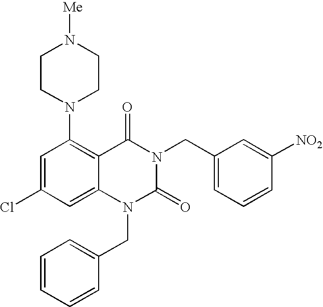 |
| 33 | 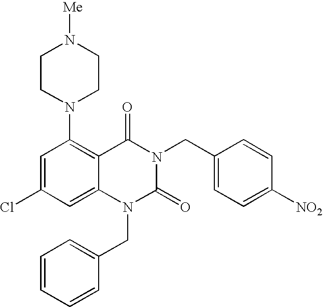 |
| 34 | 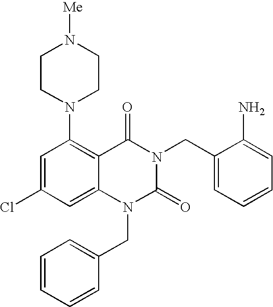 |
| 35 | 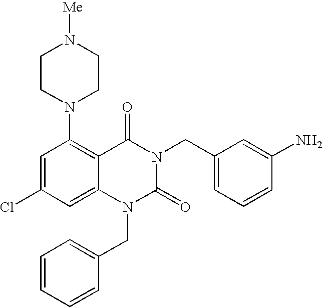 |
| 36 | 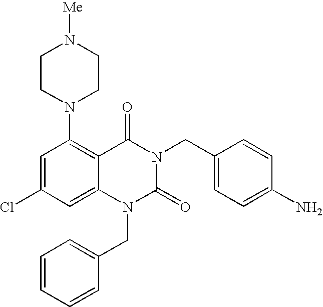 |
| 37 | 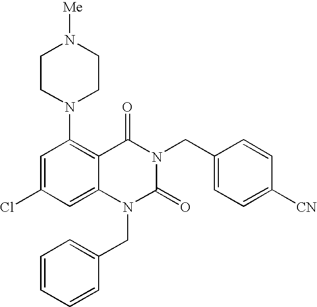 |
| 38 | 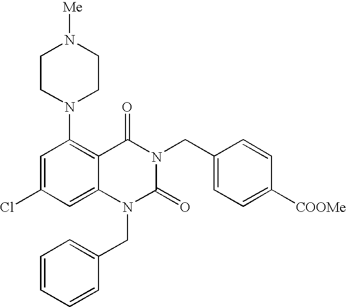 |
| 39 | 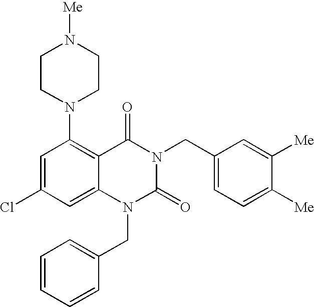 |
| 40 | 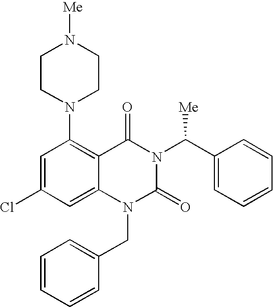 |
| 41 | 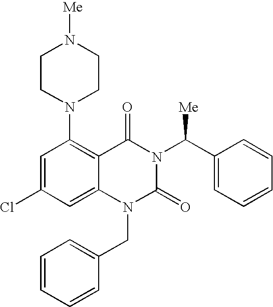 |
| 42 | 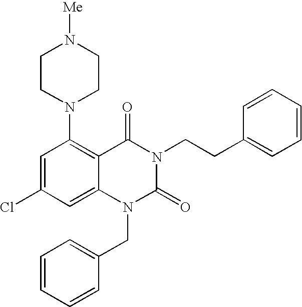 |
| 43 | 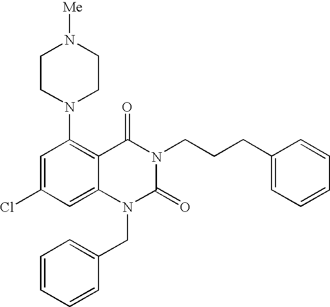 |
| 44 | 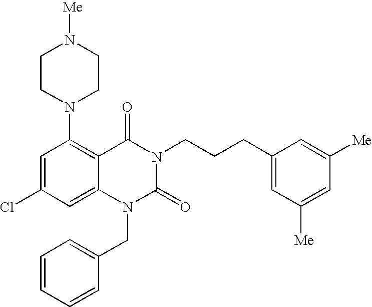 |
| 45 | 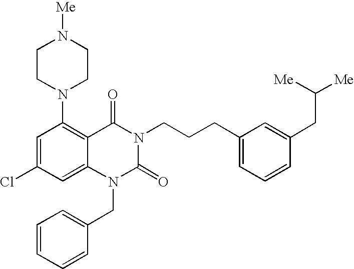 |
| 46 | 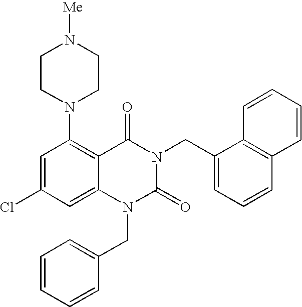 |
| 47 | 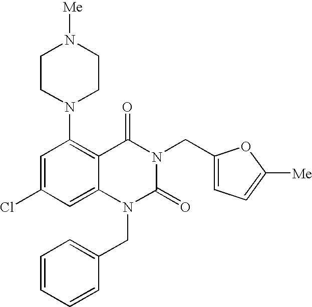 |
| 48 | 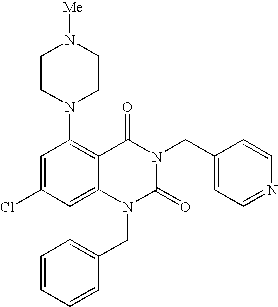 |
| 49 | 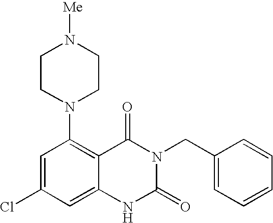 |
| 50 | 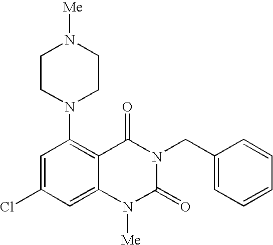 |
| 51 | 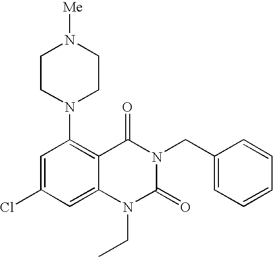 |
| 52 | 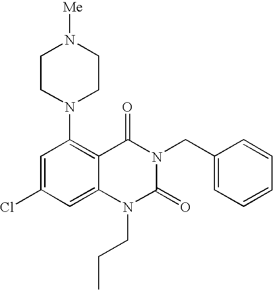 |
| 53 | 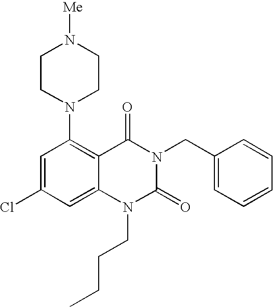 |
| 54 | 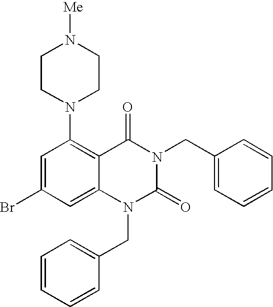 |
| 55 | 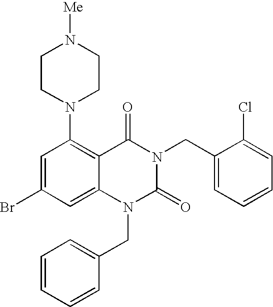 |
| 56 | 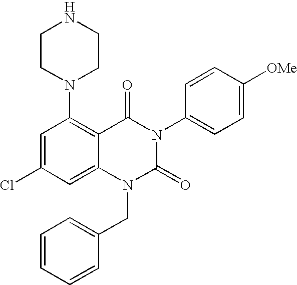 |
| 57 | 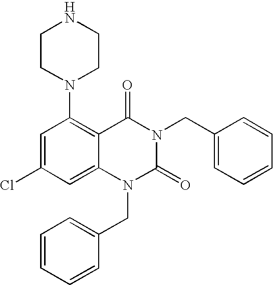 |
| 58 | 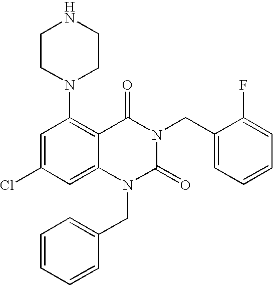 |
| 59 | 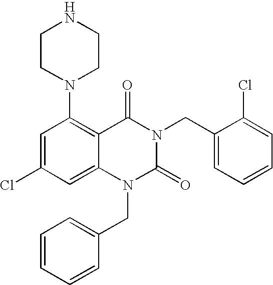 |
| 60 | 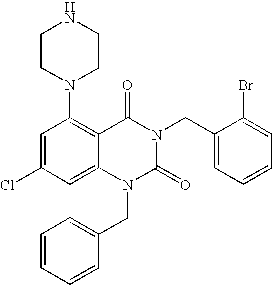 |
| 61 | 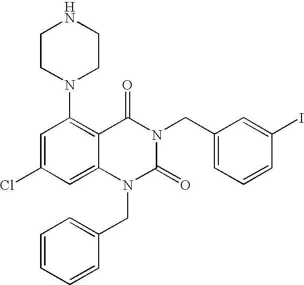 |
| 62 | 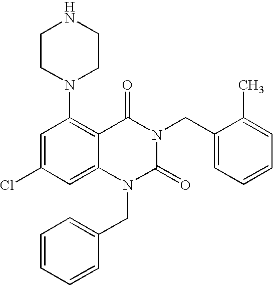 |
| 63 | 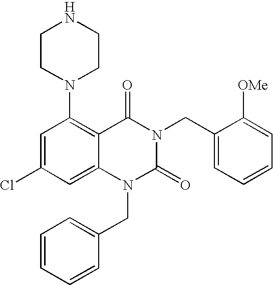 |
| 64 | 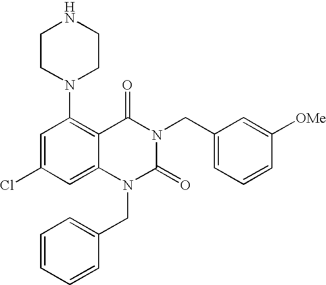 |
| 65 | 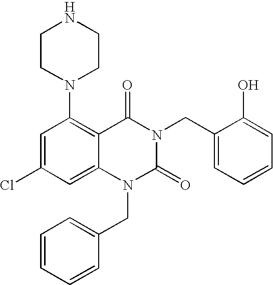 |
| 66 | 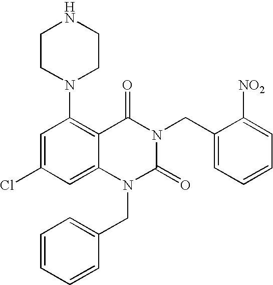 |
| 67 | 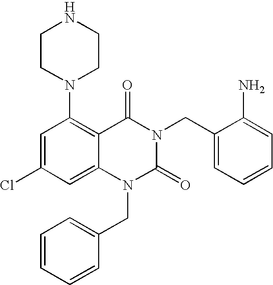 |
| 68 | 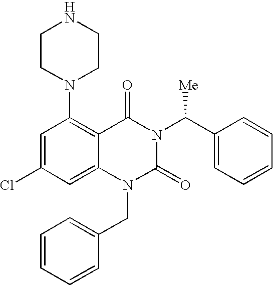 |
| 69 | 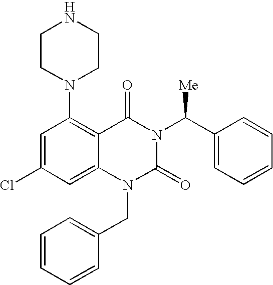 |
| 70 | 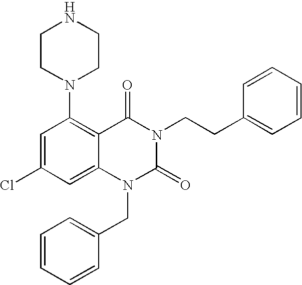 |
| 71 | 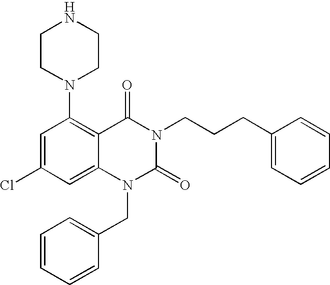 |

### Experimental Example 1

### Binding Affinity of the Compound of the Invention for 5-HT6 Receptor

1-1: Expression of Human Serotonin 5-HT6 Receptor

In order to measure the compounds of the present invention for binding strength for 5-HT6 receptors, human serotonin 5-HT6 receptor protein was expressed in insect cells, as described below.

Human 5-HT6 cDNA was cloned from the human brain cDNA library (Clontech, Palo Alto, USA) by PCR amplification using S′-TCATCTGCTTTCCCGCCACCCTAT-3′ as a forward primer and 5′-TCAGGGTCTGGGTTCTGCTCAATC-3′ as a reverse primer. Amplified cDNA fragments were introduced into pGEMT easy vector (Promega, Madison, USA) and then DNA sequencing was performed to confirm the receptor DNA sequence. A serotonin 5-HT6 clone was subcloned into insect cell expression vector BacPAK8 (Clontech). pBacPAK8/5-HT6 was transfected into each insect Sf21 cell (Clontech) and the protein expression of each 5-HT6 receptor was confirmed through SDS PAGE and receptor binding assays. Cell lysis was performed by sonication for 2 minutes at 4° C., and cell debris was removed through centrifugation for 10 min at 3,000×g. The membrane fraction was partially removed from the supernatant by centrifugation for 1 hr at 100,000×g.

1-2: Measurement of Binding Affinity for Cloned 5-HT6 Receptors

The compounds of the present invention were assayed for binding affinity for the 5-HT6 receptors prepared in Experimental Example 1-1 as follows.

A [H] LSD (lysergic acid diethylamide) binding assay was performed in 96-well plates.

The cloned receptor membranes (9 Σg/well) were mixed with [³H]LSD 1.87 nM, 10 mM MgCl₂ and 0.5 mM EDTA-containing 50 mM Tris-HCl buffer (pH 7.4) in a final volume of 0.25 ml and incubated at 37° C. for 60 min. For drug screening, testing compounds were incubated, as described above, in a reaction mixture containing 1.87 nM of [³H] LSD. After incubation, the reaction was terminated by the rapid filtration using an Inotech harvester (Inotech, Switzerland) through a Wallace GF/C glass fiber filter (Wallac, Finland) which was presoaked in 0.5% PEI, and then washed with ice-cold 50 mM Tris-HCl buffer. The filter was covered with MeltiLex, sealed in a sample bag and dried in an oven, followed by counting using a MicroBeta Plus (Wallac, Finland). Competition binding studies were carried out at 7-8 different concentrations of the test compound run in duplicate tubes, and isotherms from three assays were calculated using computerized nonlinear regression analysis (GraphPad Prism Program, San Diego, Canada) to yield median IC₅₀ (inhibitory concentration) values. Non-specific binding was determined in the presence of 10 μM methiothepin. All testing compounds were dissolved in dimethylsulfoxide (DMSO), and serially diluted to various concentrations for binding assays.

The results are summarized in Table 3, below.

TABLE 3

| Examples | IC₅₀ (nM) |
|---|---|
| 1 | 3.0 |
| 2 | 3.1 |
| 3 | 2.0 |
| 4 | 2.8 |
| 5 | 3.6 |
| 6 | 4.2 |
| 7 | 1.5 |
| 8 | 167.0 |
| 9 | 12.4 |
| 10 | 2.4 |
| 11 | 1.2 |
| 12 | 1.9 |
| 13 | 1.5 |
| 14 | 0.9 |
| 15 | 2.7 |
| 16 | 3.3 |
| 17 | 0.5 |
| 18 | 2.1 |
| 19 | 3.1 |
| 20 | 1.6 |
| 21 | 4.7 |
| 22 | 2.1 |
| 23 | 2.6 |
| 24 | 2.0 |
| 25 | 1.9 |
| 26 | 7.9 |
| 27 | 10.2 |
| 28 | 2.5 |
| 29 | 15.0 |
| 30 | 27.0 |
| 31 | 1.4 |
| 32 | 0.6 |
| 33 | 15.4 |
| 34 | 2.5 |
| 35 | 40.2 |
| 36 | 22.1 |
| 37 | 27.1 |
| 38 | 2.7 |
| 39 | 5.1 |
| 40 | 1.6 |
| 41 | 5.0 |
| 42 | 0.5 |
| 43 | 2.7 |
| 44 | 2.0 |
| 45 | 10.5 |
| 46 | 3.2 |
| 47 | 1.4 |
| 48 | 33.3 |
| 49 | >1000 |
| 50 | >1000 |
| 51 | 729.3 |
| 52 | 168.9 |
| 53 | 30.2 |
| 54 | 2.3 |
| 55 | 1.5 |
| 56 | 121.0 |
| 57 | 3.4 |
| 58 | 0.6 |
| 59 | 1.7 |
| 60 | 3.9 |
| 61 | 4.8 |
| 62 | 2.1 |
| 63 | 2.3 |
| 64 | 6.4 |
| 65 | 5.9 |
| 66 | 5.7 |
| 67 | 3.6 |
| 68 | 6.8 |
| 69 | 2.7 |
| 70 | 0.6 |
| 71 | 1.8 |
| methiothepin | 1.2 |

As seen in Table 3, most of the compounds prepared in Examples 1 to 71 were found to have good binding affinity for the 5-HT6 receptor of [H]LSD, and particularly, the compounds of Examples 7, 11˜14, 17, 20, 25, 31˜32, 40, 42, 47, 55, 58˜59 and 70˜71 were shown to have excellent affinity.

### Experimental Example 2

### Radioligand Binding Studies for 5-HT6 Receptor Selectivity

The following tests were conducted to examine how selective the compound showing excellent affinity for the 5-HT6 receptor in Experimental Example 2 was for 5-HT6 receptor in comparison with other dopamine receptors and 5-HT receptors.

2-1: Binding Assays for 5-HT Receptor Family

Radioligand bindings were performed according to the test method provided by the manufacturer of the receptor membrane (Euroscreen/BioSignal Packard Inc.). Detailed assay conditions are given in Table 3, below.

TABLE 4

| | 5-HT1a | 5-HT2a | 5-HT2c | 5-HT7 |
|---|---|---|---|---|
| Origin | Stable CHO-K1 cell strain expressing human recombinant receptors | | | |
| | (Euroscreen/BioSiganl | | | |
| Binding | 50 mM Tris-HCl | 50 mM Tris-HCl | 50 mM Tris-HCl | 50 mM Tris-HCl |
| Buffer | (pH 7.4) | (pH 7.7) | (pH 7.7) | (pH 7.4) |
| | 10 mM MgSO₄ | | 0.1% Ascorbic acid | 10 mM MgSO₄ |
| | 0.5 mM EDTA | | 10 μM Pargyline | 0.5 mM EDTA |
| | 0.1% Ascorbic acid | | | |
| Final Vol. | 250 μl | 250 μl | 250 μl | 250 μl |
| Content | 40 μg | 15 μg | 4 μg | 10 μg |
| Radioligand | [³H]8-OH-DPAT, | [³H]Ketanser | [³H] Mesulergine | [³H] LSD |
| | 0.5 nM | 1 nM | 1 nM | 3 nM |
| Non-specific | Methiothepin | Mianserin | Methiothepin | methiothepin |
| Binding | 0.5 μM | 1 μM | 10 μM | 10 μM |
| Incubation | 27° C., | 37° C., | 37° C., | 27° C., |
| | 60 min | 15 min | 30 min | 120 min |
| Filtration | GF/C, 0.3% PEI | GF/C, 0.05% Brij | GF/C, 1% BSA | GF/C, 0.3% PEI |

2-2: Binding Assays for Dopamine Receptor Family

Radioligand binding assays for dopamine receptor family (D₁, D₂ and D₃) were performed according to the protocols provided by the manufacturer of receptor membranes (BioSignal Packard Inc., Montreal, Canada). The radioligands used were [³H] spiperone (for hD_{2L} and hD₃ receptors, 1 nM) and [³H] YM-09151-2 (hD_{4.2} receptor, 0.06 nM). Briefly, the buffer used in the D₁, D₂ and D₃ receptor binding assay was 50 mM Tris-HCl (pH 7.4), containing 10 mM MgCl₂ and 1 mM EDTA, or 50 mM Tris-HCl (pH 7.4), containing 5 mM MgCl₂, 5 mM EDTA, 5 mM KCl, 1.5 mM CaCl₂ and 120 mM NaCl. For [³H] YM-09151-2 receptor binding assays, 50 mM Tris-HCl (pH 7.4) containing 5 mM MgCl₂, 5 mM EDTA, 5 mM KCl and 1.5 mM CaCl₂, was used as a buffer. Nonspecific binding was determined with haloperidol (10 μM) for D₂ and D₃ and clozapine (10 μM) for D₄ receptors.

Competition binding studies were carried out with 7-8 concentrations of the test compound run in duplicate tubes, and isotherms from three assays were calculated by computerized nonlinear regression analysis (GraphPad Prism Program, San Diego, Canada) to yield median inhibitory concentration (IC₅₀) values.

The selectivity of compounds according to the present invention for the dopamine and serotonin receptor subtypes is summarized in Table 5, below.

TABLE 5
Binding affinity of the Compounds for Serotonin Receptor Subtypes and Dopamine

| | Binding affinity IC₅₀ (nM) | | | | | | | | |
|---|---|---|---|---|---|---|---|---|---|
| Examples | 5-HT6 | 5-HT1a | 5-HT2a | 5-HT2c | 5-HT7 | D₁ | D₂ | D₃ | D₄ |
| 3 | 2.0 | >10000 | 718 | 3759 | >10000 | ND | >10000 | 2109 | >10000 |
| 7 | 1.5 | 2157 | 1139 | 1707 | 2560 | ND | 2761 | 606 | >10000 |
| 10 | 2.4 | 4589 | 1690 | 4763 | 6784 | 5560 | 4350 | 1548 | >10000 |
| 11 | 1.2 | 1510 | 463 | 176 | 4841 | ND | >10000 | 2664 | >10000 |
| 12 | 1.9 | 718 | 622 | 1063 | 1671 | ND | >10000 | 490 | >10000 |
| 13 | 1.5 | 2773 | 1388 | 1802 | 8635 | ND | >10000 | 2375 | >10000 |
| 14 | 0.9 | 1076 | 394 | 728 | 5942 | ND | 3316 | 480 | >10000 |
| 17 | 0.5 | 3279 | 169 | 512 | 2184 | ND | >10000 | 138 | >10000 |
| 18 | 2.1 | 1781 | 1378 | 748 | 1882 | ND | 7323 | 339 | >10000 |
| 19 | 3.1 | 3060 | 1104 | 321 | 2206 | ND | 7743 | 1011 | >10000 |
| 20 | 1.6 | 3534 | 912 | 1055 | 1920 | ND | >10000 | 615 | >10000 |
| 22 | 2.1 | 5778 | 663 | 216 | 3972 | ND | >10000 | 1539 | >10000 |
| 24 | 2.0 | 3213 | 733 | 599 | >10000 | ND | >10000 | 435 | >10000 |
| 25 | 1.9 | 7076 | 916 | 925 | >10000 | ND | >10000 | 1330 | >10000 |
| 31 | 1.4 | 2244 | 49 | 952 | >10000 | ND | >10000 | 1240 | >10000 |
| 32 | 0.6 | 3804 | 1797 | 1565 | >10000 | ND | >10000 | 276 | >10000 |
| 40 | 1.6 | 4348 | 266 | 477 | 7425 | 2706 | >10000 | 983 | >10000 |
| 41 | 5.0 | >10000 | 160 | 865 | 2736 | >10000 | 744 | 362 | >10000 |
| 42 | 0.5 | 1305 | 244 | 430 | 7031 | 3449 | 2495 | 638 | >10000 |
| 43 | 2.7 | 2643 | 892 | 1424 | 7764 | 961 | 5668 | 1754 | >10000 |
| 44 | 2.0 | 2778 | 197 | 2646 | >10000 | ND | 987 | 73 | >10000 |
| 47 | 1.4 | 1137 | 830 | 465 | 8743 | ND | >10000 | 2815 | >10000 |
| 54 | 2.3 | 5013 | 376 | 646 | >10000 | ND | >10000 | 554 | >10000 |
| 55 | 1.5 | 2717 | 130 | 252 | 5143 | ND | >10000 | 1441 | >10000 |
| 58 | 0.6 | 2854 | 220 | 540 | >10000 | ND | >10000 | 3128 | >10000 |
| 59 | 1.7 | 2159 | 132 | 198 | 7325 | ND | >10000 | 381 | >10000 |
| 69 | 2.7 | 5871 | 293 | 429 | 7888 | 8616 | >10000 | 1797 | >10000 |
| 70 | 0.6 | 2910 | 401 | 752 | 7705 | 5712 | 7437 | 2839 | >10000 |
| 71 | 1.8 | 891 | 733 | 1098 | 3046 | 2320 | 2723 | 1204 | >10000 |
| SB-271046 | 0.8 | 3498 | 313 | 4651 | 3963 | 9138 | >10000 | 4119 | >10000 |

* N.D.: IC₅₀ not determined

As shown in Table 5, the significantly low IC₅₀ values of the compounds according to the present invention for 5-HT6 receptors oilier than 5-HT receptors are evidence of very high selectivity for 5-HT6 receptors compared with 5-HT receptors. This is true of dopamine receptor family. That is, the compounds of the present invention show far smaller IC₅₀ values for 5-HT6 receptors than for the dopamine receptor family, evincing a greater selectivity for 5-HT6 receptors than for the dopamine receptor family.

Therefore, the compounds of the present invention have high selectivity for 5-HT6 receptors.

### Experimental Example 3

### In vitro Functional Studies

Using the Rutledge et al. method, disclosed by MDSPS (MDS Pharma Service, WO 1019612, Taiwan), adenylyl cyclase was measured for activity in HeLa cells transfected with human 5-HT6 receptor.

Detailed conditions for this assay are summarized in Table 6, below. The culture medium was HBSS (Hanks' Balanced Salt Solution, pH 7.4) containing 1 mM MgCl₂, 1 mM CaCl₂ and 100 mM 1-methyl-3-isobutylxanthine. The cells were incubated in the mixture in the presence of the enzyme and the compounds according to the present invention. Following 20 minutes of incubation at 37° C., intracellular cAMP levels were measured through EIA (enzyme-immunoassay), and a compound showing 50% or higher inhibition against serotonin(5-HT)-stimulated cAMP accumulation was classified as an antagonist. Methiothepin, known as an antagonist of 5-HT6, was used as a control.

TABLE 6
Assay Conditions

| | |
|---|---|
| Target | Human HeLa cell |
| Vehicle | 0.4% DMSO |
| Culture time and | 20 min, 37° C. |
| temp. | |
| Culture buffer | HBSS (pH. 7.4), 1 mM MgCl₂, |
| | 1 mM CaCl₂, 100 mM IBMX |
| Assay method | EIA for accumulated cAMP level |
| Antagonist Standard | cAMP increase ≧50% by serotonin |
| | (0.3 μM) inhibition |
| Effector Standard | Serotonin-Induced cAMP Accumulation by ≧50% |

The results are shown in FIG. 1.

As seen in FIG. 1, the effector serotonin stimulated cAMP accumulation in the HeLa cells, in which 5-HT6 receptors were selectively expressed (EC50=16.9 nM), but the compound of Example 40 and methiothepin inhibited serotonin-stimulated cAMP accumulation in a dose-dependent manner. Particularly, the compound of Example 40 (0.001, 0.01, 0.1, 1 and 10 μM) was found to be 0, 35, 95, 100 and 100% inhibitory against the cAMP increase induced by 0.3 μM of serotonin (5-HT, respectively, demonstrating its excellent antagonistic activity. In addition, the compound of Example 40 did not show any cytotoxicity at all.

Among the compounds according to the present invention, those of examples 1 and 13, having excellent receptor affinity and typical skeletal structures, were identified as 5-HT6 receptor antagonists and showed weaker antagonist activity than methiothepin, a nonselective antagonist. However, they have higher potential as medicine because of their excellent selectivity. The inhibitory effects of example 1, 13 and methiothepin on serotonin (5-HT)-stimulated cAMP accumulation, determined using the HeLa cell line, are shown in FIG. 1.

### Experimental Example 4

### In Vivo Effect on Apomorphine-Induced PPI Disruption in Rat

A prepulse inhibition test was carried out in order to examine whether the compounds of the present invention act as antipsychotic agents, as follows.

In this regard, rats were assayed for startle response in an SR-LAB startle chamber (San Diego Instruments, San Diego, USA).

Testing was conducted in a startle device consisting of a Plexiglas cylinder (40 mm in diameter) mounted on a Plexiglas platform in a ventilated, sound-attenuated cubicle. The background noise in each chamber was 68 dB. Movements within the cylinder were detected and transduced by a piezoelectric accelerometer attached to the Plexiglas base and digitized and stored by a computer. An acoustic noise burst was reproduced through a high-frequency loudspeaker located 24 cm above the experimental animal.

Behavior tests were conducted in a light condition from 10:00 am to 5:00 pm according to a modified version of the Mansbach method [Mansbach R S, Brooks E W, Sanner M A, Zorn S H, Selective dopamine D₄ receptor antagonists reverse apomorphine-induced blockade of prepulse inhibition., Psychopharmacology(Berl), 135:194-200, 1998]. Each startle section was initiated with a 5 min acclimation period for the background noise of 68 dB. The following different trial types were then presented for each experiment: 40 ms-long bursts of 120 dB broadband (P: pulse alone trial), 20 ms-long bursts of a single? frequency 10 dB louder than the background noise 100 ms before P (pP: prepulse+pulse trial), 40 ms-long bursts of 78 dB broadband (prepulse alone trial), and non-stimulus trial (baseline). 8 trials were presented in a pseudorandom order for each type so that 32 trials were conducted in total and the average intertial interval (ITI) was 15 msec. 5 pulse alone trials were additionally presented for the start and end of each trial, which were not included in the analysis. PPI was expressed as a percentage of the prepulse startle amplitude, decreased compared to no prepulse trial, in accordance with the following mathematical formula 1.

$$PPI ( \% ) = ( 1 - \frac{Prepulse Startle Amplitude}{Pulse Startle Amplitude} ) \times 100 \qquad [ Mathematical Formula 1$$

The compound of Example 40 or 68, SB-271046 or a carrier was injected (i.p.) into rats 30 min before the injection of apomorphine (3 mg/kg, i.p.). 30 min after the administration of apomorphine (2 mg/kg, i.p.) for testing, the rats were placed in startle chambers. For use, compounds of Examples 40 and 68 or SB-271046 were suspended in 3% Tween 80.

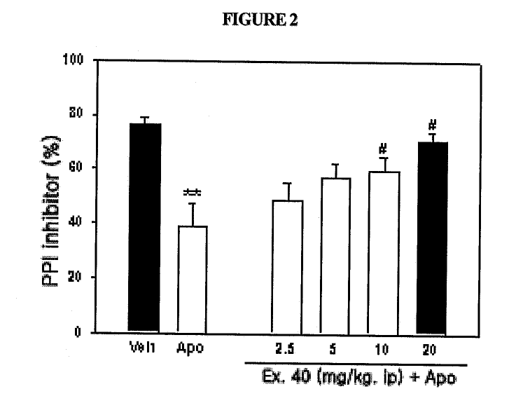

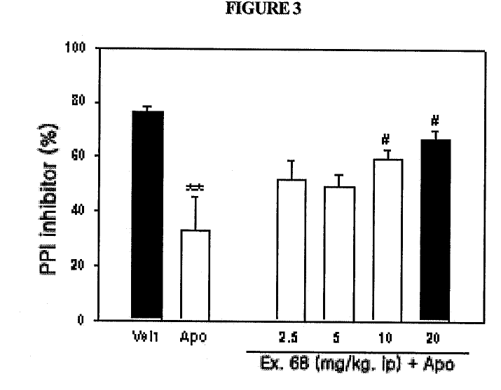

For comparison between the treated group and the control group, resultant statistical data were analyzed using one-way ANOVA and a Dunnett's post-hoc test. Deviation was considered significance at p< 0.05. Statistical analysis was performed using SigmaStat software (Jandel Co., San Rafael, Calif.). Data were represented as mean±SEM. The results are graphed in FIGS. 2 and 3.

As seen in FIGS. 2 and 3, no significant effects were detected in the group administered (i.p.) with the compound of Example 40 (2.5, 5, 10 and 20 mg/kg) or Example 60 (2.5, 5, 10 and 20 mg/kg) alone in comparison with the control, which was administered with a vehicle alone. However, pretreatment with Example 40 (P=0.005), Example 68 (P=0.021) or SB-271046 (P<0.05) was found to inhibit apomorphine (2 mg/kg, i.p.)-induced PPI disruption, demonstrating its antipsychotic activity. In addition, when compared with a positive control administered with apomorphine, the administration of the compound of Example 40 or 68 30 min before apomorphine administration did not induce significant difference in mean startle amplitude. However, apomorphine-induced PPI disruption was not reversed by SB-271046 (2.5, 5, 10 and 20 mg/kg, i.p.).

### Experimental Example 5

### Effect of the Compounds on Rotarod Deficit in Mice

In order to examine the effects of the compounds of the present invention on the central nervous system and behavior, a rotarod test was performed as follows.

Mice were placed on a plastic rod having a node 1 inch in diameter (Ugo-Basile, Milano, Italy), followed by rotating the rod at a speed of 6 rpm. 30, 60, 90 and 120 min after the administration of test compounds, the mice that fell from the rotating rod within 1 min were counted to determine rotarod deficits (%) (Dunham et al., 1957). A median neurotoxic dose (TD₅₀) was determined as the dose at which 50% of the total number of animals showed a rotarod

) deficit. Compounds of Examples 40 and 60 and SB-271046 were suspended in 3% Tween 80, and the suspension thus obtained was administered (i.p.) 30 min before the rotarod testing. The results are summarized in Table 7, below.

TABLE 7
Effects of the compounds on Rotarod Deficit in Mice

| | Rotarod Deficit TD₅₀ | Lethal Degrees LD₅₀ |
|---|---|---|
| EXAMPLES | (mg/kg, ip) | (mg/kg, ip) |
| 40 | 139 | >300 |
| 68 | 122 | 152 |
| SB-271046 | 112 | 123 |

Administration with the individual compounds of Example 40 and 68 (150 mg/kg, i.p.) alone induced 75% and 100% rotarod dysfunction, respectively, 120 min after the treatment. In addition, as seen in Table 7, their TD₅₀ values were calculated to be 139 mg/kg and 122 mg/kg, respectively, with the concomitant production of extrapyramidal side effects lower than those of SB-271046, which showed a TD₅₀ of 112 mg/kg. Hence, the compounds of Examples 40 and 68 are safer for mice than is SB-271046.

Pharmaceutical preparations of the present invention are illustrated in the following examples.

### Preparation Example 1

### Preparation of Pharmaceutical Compositions

1-1: Preparation of Powder

| | |
|---|---|
| Compound of the present invention, pharmaceutically | 2 g |
| acceptable salt or prodrug thereof | |
| Lactose | 1 g |

These ingredients were mixed and loaded in an airtight bag to obtain a powder.

1-2: Preparation of Tablet

| | | |
|---|---|---|
| Compound of the present invention, pharmaceutically | 100 | mg |
| acceptable salt or prodrug thereof | | |
| Corn starch | 100 | mg |
| Lactose | 100 | mg |
| Mg stearate | 2 | mg |

These ingredients were mixed, and the resulting mixture was tabletted in a typical manner to afford a tablet.

1-3: Preparation of Capsule

| | | |
|---|---|---|
| Compound of the present invention, pharmaceutically | 100 | mg |
| acceptable salt or prodrug thereof | | |
| Corn starch | 100 | mg |
| Lactose | 100 | mg |
| Mg stearate | 2 | mg |

These ingredients were mixed and the resulting mixture was loaded into a gelatin capsule in a typical manner to afford a capsule.

### INDUSTRIAL APPLICABILITY

As described hereinbefore, the novel substituted-1H-quinazoline-2,4-dione derivatives according to the present invention and the pharmaceutical composition containing the same as an active ingredient show inhibitory activity against the 5-HT6 receptor, thereby being applicable to the treatment of 5-HT6 receptor-mediated diseases of the central nervous central system, and particularly to the treatment of cognitive disorders, Alzheimer's disease, anxiety, depression, schizophrenia, stress disorder, panic disorder, phobic disorder, obsessive compulsive disorder (OCD), post-traumatic-stress syndrome, psychosis, immunity decrease, mental illness, paraphrenia, mania, compulsive disorder, migraine, drug addiction, alcohol addiction, obesity, eating disorders, and sleep disorder.

Although the preferred embodiments of the present invention have been disclosed for illustrative purposes, those skilled in the art will appreciate that various modifications, additions and substitutions are possible, without departing from the scope and spirit of the invention as disclosed in the accompanying claims.

1. A substituted-1H-quinazoline-2,4-dione derivative represented by the following Chemical Formula 1, or a pharmaceutically acceptable salt thereof: 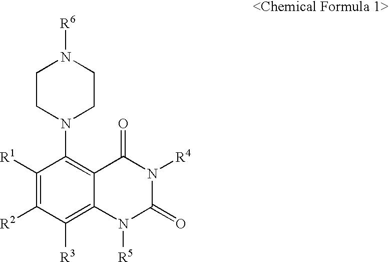
wherein,
R¹, R² and R³ are independently selected from the group consisting of hydrogen, halogen, amino, cycloamino, nitro, cyano, alkyl, haloalkyl, alkoxy, haloalkoxy, piperidinyl and N-methyl piperidinyl;
R⁴ is selected from the group consisting of hydrogen, alkyl, cycloalkyl, haloalkyl, aryl, heteroaryl, aralkyl, heteroarylalkyl, alkoxy, aryloxy, acylamino, arylsulfonylamino, arylsulfonylureido, alkylcarboxylate, arylcarboxylate, aralkylcarboxylate, alkylureido and arylureido;
R³ is selected from the group consisting of hydrogen, alkyl, cycloalkyl, haloalkyl, aryl, arylalkyl, heteroaryl and heteroarylalkyl; and
R⁶ is selected from the group consisting of hydrogen, alkyl and aryl group.

2. The substituted-1H-quinazoline-2,4-dione derivative or a pharmaceutically acceptable salt thereof according to claim 1, wherein
R¹, R² and R³ are independently or optionally hydrogen, chloro, bromo or methoxy,
R⁴ is hydrogen, alkyl, aryl, arylalkyl, heteroaryl or heteroarylalkyl,
R⁵ is hydrogen, methyl, ethyl, propyl, n-butyl or benzyl, and
R⁶ is hydrogen or methyl.

3. The substituted-1H-quinazoline-2,4-dione derivative or a pharmaceutical acceptable salt thereof according to claim 1, wherein, the alkyl of R⁴ is methyl, ethyl, propyl, n-butyl, cyclohexylmethyl or octyl; the aryl of R⁴ is phenyl or methoxyphenyl; the arylalkyl of R⁴ is benzyl, phenethyl, (R)-1-phenyl-ethyl, (S)-1-phenyl-ethyl, phenylpropyl or naphthalenylmethyl; and the heteroarylalkyl of R⁴ is pyridinylmethyl or furanylmethyl, substituted with one or more substituents selected from the group consisting of hydrogen, fluoro, chloro, bromo, iodo, nitro, amino, cyano, hydroxy, methyl carboxylate, methyl, methoxy, and isobutyl.

4. The substituted-1H-quinazoline-2,4-dione derivative or a pharmaceutically acceptable salt thereof according to claim 1, wherein the substituted-1H-quinazoline-2,4-dione derivative is selected from the group consisting of:
(1) 1-benzyl-7-chloro-3-methyl-5-(4-methyl-piperazin-1-yl)-1H-quinazoline-2,4-dione;
(2) 1-benzyl-7-chloro-3-ethyl-5-(4-methyl-piperazin-1-yl)-1H-quinazoline-2,4-dione;
(3) 1-benzyl-7-chloro-5-(4-methyl-piperazin-1-yl)-3-propyl-1H-quinazoline-2,4-dione;
(4) 1-benzyl-3-butyl-7-chloro-5-(4-methyl-piperazin-1-yl)-1H-quinazoline-2,4-dione;
(5) 1-benzyl-7-chloro-3-(3-methyl-butyl)-5-(4-methyl-piperazin-1-yl)-1H-quinazoline-2,4-dione;
(6) 1-benzyl-7-chloro-3-cyclohexylmethyl-5-(4-methyl-piperazin-1-yl)-1H-quinazoline-2,4-dione;
(7) 1-benzyl-7-chloro-5-(4-methyl-piperazin-1-yl)-3-octyl-1H-quinazoline-2,4-dione;
(8) 1-benzyl-7-chloro-5-(4-methyl-piperazin-3-yl)-3-phenyl-1H-quinazoline-2,4-dione;
(9) 1-benzyl-7-chloro-3-(4-methoxy-phenyl)-5-(4-methyl-piperazin-1-yl)-1H-quinazoline-2,4-dione;
(10) 1,3-dibenzyl-7-chloro-5-(4-methyl-piperazin-1yl)-1H-quinazoline-2,4-dione;
(11) 1-benzyl-7-chloro-3-(2-fluoro-benzyl)-5-(4-methyl-piperazin-1-yl)-1H-quinazoline-2,4-dione;
(12) 1-benzyl-7-chloro-3-(3-fluoro-benzyl)-5-(4-methyl-piperazin-1-yl)-1H-quinazoline-2,4-dione;
(13) 1-benzyl-7-chloro-3-(4-fluoro-benzyl)-5-(4-methyl-piperazin-1-yl)-1H-quinazoline-2,4-dione;
(14) 1-benzyl-3-(2-bromo-benzyl)-7-chloro-5-(4-methyl-piperazin-1-yl)-1H-quinazoline-2,4-dione;
(15) 1-benzyl-3-(3-bromo-benzyl)-7-chloro-5-(4-methyl-piperazin-1-yl)-1H-quinazoline-2,4-dione;
(16) 1-benzyl-3-(4-bromo-benzyl)-7-chloro-5-(4-methyl-piperazin-1-yl)-1H-quinazoline-2,4-dione;
(17) 1-benzyl-7-chloro-3-(2-chloro-benzyl)-5-(4-methyl-piperazin-1-yl)-1H-quinazoline-2,4-dione;
(18) 1-benzyl-7-chloro-3-(3-chloro-benzyl)-5-(4-methyl-piperazin-3-yl)-1H-quinazoline-2,4-dione;
(19) 1-benzyl-7-chloro-3-(4-chloro-benzyl)-5-(4-methyl-piperazin-1-yl)-1H-quinazoline-2,4-dione;
(20) 1-benzyl-7-chloro-3-(3-iodo-benzyl)-5-(4-methyl-piperazin-1-yl)-1H-quinazoline-2,4-dione;
(21) 1-benzyl-7-chloro-3-(4-iodo-benzyl)-5-(4-methyl-piperazin-1-yl)-1H-quinazoline-2,4-dione;
(22) 1-benzyl-7-chloro-3-(2-methyl-benzyl)-5-(4-methyl-piperazin-1-yl)-1H-quinazoline-2,4-dione;
(23) 1-benzyl-7-chloro-3-(3-methyl-benzyl)-5-(4-methyl-piperazin-1-yl)-1H-quinazoline-2,4-dione;
(24) 1-benzyl-7-chloro-3-(4-methyl-benzyl)-5-(4-methyl-piperazin-1-yl)-1H-quinazoline-2,4-dione;
(25) 1-benzyl-7-chloro-3-(2-methoxy-benzyl)-5-(4-methyl-piperazin-1-yl)-1H-quinazoline-2,4-dione;
(26) 1-benzyl-7-chloro-3-(3-methoxy-benzyl)-5-(4-methyl-piperazin-1yl)-1H-quinazoline-2,4-dione;
(27) 1-benzyl-7-chloro-3-(4-methoxy-benzyl)-5-(4-methyl-piperazin-1-yl)-1H-quinazoline-2,4-dione;
(28) 1-benzyl-7-chloro-3-(2-hydroxy-benzyl)-5-(4-methyl-piperazin-1-yl)-1H-quinazoline-2,4-dione;
(29) 1-benzyl-7-chloro-3-(3-hydroxy-benzyl)-5-(4-methyl-piperazin-1-yl)-1H-quinazoline-2,4-dione;
(30) 1-benzyl-7-chloro-3-(4-hydroxy-benzyl)-5-(4-methyl-piperazin-1-yl)-1H-quinazoline-2,4-dione;
(31) 1-benzyl-7-chloro-5-(4-methyl-piperazin-1-yl)-3-(2-nitro-benzyl)-1H-quinazoline-2,4-dione;
(32) 1-benzyl-7-chloro-5-(4-methyl-piperazin-1-yl)-3-(3-nitro-benzyl)-1H-quinazoline-2,4-dione;
(33) 1-benzyl-7-chloro-5-(4-methyl-piperazin-1-yl)-3-(4-nitro-benzyl)-1H-quinazoline-2,4-dione;
(34) 3-(2-amino-benzyl)-1-benzyl-7-chloro-5-(4-methyl-piperazin-1-yl)-1H-quinazoline-2,4-dione;
(35) 3-(3-amino-benzyl)-1-benzyl-7-chloro-5-(4-methyl-piperazin-1-yl) 1H-quinazoline-2,4-dione;
(36) 3-(4-amino-benzyl)-1-benzyl-7-chloro-5-(4-methyl-piperazin-1-yl)-1H-quinazoline-2,4-dione;
(37) 4-[1-benzyl-7-chloro-5-(4-methyl-piperazin-1-yl)-2,4-dioxo-1,4-dihydro-2H-quinazoline-3-ylmethyl]-benzonitrile;
(38) 4-[1-benzyl-7-chloro-5-(4-methyl-piperazin-1-yl)-2,4-dioxo-1,4-dihydro-2H-quinazoline-3-ylmethyl]-benzoic acid methyl ester;
(39) 1-benzyl-7-chloro-3-(3,4-dimethyl-benzyl)-5-(4-methyl-piperazin-1-yl)-1H-quinazoline-2,4-dione;
(40) 1-benzyl-7-chloro-5-(4-methyl-piperazin-1yl)-3-[(R)-1-phenyl-ethyl]-1H-quinazoline-2,4-dione;
(41) 1-benzyl-7-chloro-5-(4-methyl-piperazin-1yl)-3-[(S)-1-phenyl-ethyl]-1H-quinazoline-2,4-dione;
(42) 1-benzyl-7-chloro-5-(4-methyl-piperazin-1-yl)-3-phenethyl-1H-quinazoline-2,4-dione;
(43) 1-benzyl-7-chloro-5-(4-methyl-piperazin-1-yl)-3-(3-phenyl-propyl)-1H-quinazoline-2,4-dione;
(44) 3-benzyl-7-chloro-3-[3-(3,5-dimethyl-phenyl)-propyl]-5-(4-methyl-piperazin-1-yl)-1H-quinazoline-2,4-dione;
(45) 1-benzyl-7-chloro-3-[3-(3-isobutyl-phenyl)-propyl]-5-(4-methyl-piperazin-1-yl)-1H-quinazoline-2,4-dione;
(46) 1-benzyl-7-chloro-5-(4-methyl-piperazin-1-yl)-3-naphthalen-1-ylmethyl-1H-quinazoline-2,4-dione;
(47) 1-benzyl-7-chloro-3-(5-methyl-furan-2-ylmethyl)-5-(4-methyl-piperazin-3-yl)-1H-quinazoline-2,4-dione;
(48) 1-benzyl-7-chloro-5-(4-methyl-piperazin-1-yl)-3-pyridin-4-ylmethyl-1H-quinazoline-2,4-dione;
(49) 3-benzyl-7-chloro-5-(4-methyl-piperazin-1-yl)-1H-quinazoline-2,4-dione;
(50) 3-benzyl-7-chloro-1-methyl-5-(4-methylpiperazin-1-yl)-1H-quinazoline-2,4-dione;
(51) 3-benzyl-7-chloro-1-ethyl-5-(4-methyl-piperazin-1-yl)-1H-quinazoline-2,4-dione;
(52) 3-benzyl-7-chloro-5-(4-methyl-piperazin-1-yl)-1-propyl-1H-quinazoline-2,4-dione;
(53) 3-benzyl-1-butyl-7-chloro-5-(4-methyl-piperazin-1-yl)-1H-quinazoline-2,4-dione;
(54) 1,3-dibenzyl-7-bromo-5-(4-methyl-piperazin-1-yl)-1H-quinazoline-2,4-dione;
(55) 1-benzyl-7-bromo-3-(2-chloro-benzyl)-5-(4-methyl-piperazin-1-yl)-1H-quinazoline-2,4-dione;
(56) 1-benzyl-7-chloro-3 (4-methoxy-phenyl)-5-piperazin-1-yl-1H-quinazoline-2,4-dione;
(57) 1,3-dibenzyl-7-chloro-5-piperazin-1-yl-1H-quinazoline-2,4-dione;
(58) 1-benzyl-7-chloro-3-(2-fluoro-benzyl)-5-piperazin-1-yl-1H-quinazoline-2,4-dione;
(59) 1-benzyl-7-chloro-3-(2-chloro-benzyl)-5-piperazin-1-yl-1H-quinazoline-2,4-dione;
(60) 1-benzyl-3-(2-bromo-benzyl)-7-chloro-5-piperazin-1-yl-1H-quinazoline-2,4-dione;
(61) 1-benzyl-5-chloro-3-(3-iodo-benzyl)-7-piperazin-1-yl-1H-quinazoline-2,4-dione;
(62) 1-benzyl-5-chloro-3-(2-methoxy-benzyl)-7-piperazin-1-yl-1H-quinazoline-2,4-dione;
(63) 1-benzyl-7-chloro-3-(2-methoxy-benzyl)-5-piperazin-1-yl-1H-quinazoline-2,4-dione;
(64) 1-benzyl-7-chloro-3-(3-methoxy-benzyl)-5-piperazin-1-yl-1H-quinazoline-2,4-dione;
(65) 1-benzyl-7-chloro-3-(2-hydroxy-benzyl)-5-piperazin-1-yl-1H-quinazoline-2,4-dione;
(66) 1-benzyl-7-chloro-3-(2-nitro-benzyl)-5-piperazin-1-yl-1H-quinazoline-2,4-dione;
(67) 3-(2-amino-benzyl)-1-benzyl-7-chloro-5-piperazin-1-yl-1H-quinazoline-2,4-dione;
(68) 1-benzyl-7-chloro-3-[(R)-1-phenyl-ethyl]-5-piperazin-1-yl-1H-quinazoline-2,4-dione;
(69) 1-benzyl-7-chloro-3-[(S)-1-phenyl-ethyl]-5-piperazin-1-yl-1H-quinazoline-2,4-dione;
(70) 1-benzyl-7-chloro-3-phenethyl-5-piperazin-1-yl-1H-quinazoline-2,4-dione; and
(71) 1-benzyl-7-chloro-3-(3-phenyl-propyl)-5-piperazin-1-yl-1H-quinazoline-2,4-dione.

5. A method for preparing the novel substituted-1H-quinazoline-2,4-dione derivative of Chemical Formula 1 of claim 1, as delineated in the following Reaction Scheme 1, comprising:
(a) reacting an anthranilic anhydride of Chemical Formula 2 with an amine compound of Chemical Formula 3 to afford intermediate I;
(b) cyclizing the intermediate I of step (a) into intermediate II;
(c) reacting the intermediate II of step (b) with a compound of Chemical Formula 4 to afford intermediate III; and
(d) reacting the intermediate III of step (c) with an amine compound to produce a substituted-1H-quinazoline-2,4-dione derivative of Chemical Formula 1. 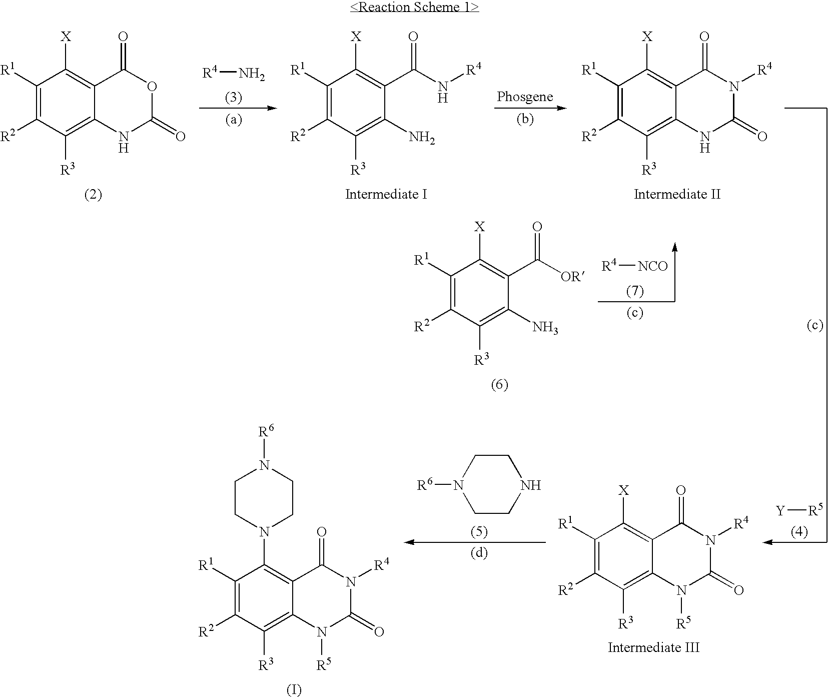
(wherein, R¹-R⁶ are as defined in Chemical Formula 1 of claim 1, X is a fluorine, chlorine, bromine or iodine atom or trifluoroacetate, and Y is chloro, bromo, iodo, methanesulfonate or p-toluenesulfonate.)

6. The method according to claim 5, wherein the reaction between anthranilic anhydride (2) and the compound of chemical formula (3) in step (a) is conducted through nucleophilic substitution and decarboxylation to produce open Intermediate I.

7. The method according to claim 5, wherein the cyclization in step (b) is conducted by reacting the intermediate I of step (a) with diphosgene or triphosgene.

8. The method according to claim 5, wherein the reaction in step (c) is conducted by introducing R⁵ on N(1) through nucleophilic substitution between the intermediate II of step (b) and the compound of chemical formula 4.

9. The method according to claim 5, wherein the amine compound (5) of step (d) is piperazine or N-methylpiperazine.

10. The method according to claim 5, comprising cyclizing a compound of chemical formula 6 with an isocyanate compound of chemical formula 7 into intermediate II, instead of steps (a) and (b).

11. A pharmaceutical composition, comprising the substituted-1H-quinazoline-2,4-dione derivative of claim 1, a pharmaceutically acceptable salt or a prodrug thereof as an active ingredient functioning as a 5-HT6 serotonin receptor antagonist.

12. A pharmaceutical composition for the treatment of central nervous system diseases, comprising the substituted-1H-quinazoline-2,4-dione derivative of claim 1, a pharmaceutically acceptable salt or prodrug thereof as an active ingredient.

13. The pharmaceutical composition according to claim 12, wherein the central nervous system disease is selected from the group consisting of cognitive disorders. Alzheimer's disease, anxiety, depression, schizophrenia, stress disorder, panic disorder, phobic disorder, obsessive compulsive disorder (OCD), post-traumatic-stress syndrome, immunosuppression, psychosis, immunity decrease, mental illness, paraphrenia, mania, compulsive disorder, migraines, drag addiction, alcohol addiction, obesity, eating disorders, and sleep disorder.

14. A method for treating the central nervous system disorders in a subject comprising administering a pharmaceutically effective amount of the substituted-1H-quinazoline-2,4-dione derivative of claim 1 or a pharmaceutically acceptable salt thereof to the subject in need thereof.

15. The method according to claim 14, wherein the central nervous system disorder is selected from the group consisting of cognitive disorders. Alzheimer's disease, anxiety, depression, schizophrenia, stress disorder, panic disorder, phobic disorder, obsessive compulsive disorder (OCD), post-traumatic-stress syndrome, immunosuppression, psychosis, immunity decrease, mental illness, paraphrenia, mania, compulsive disorder, migraines, drug addiction, alcohol addiction, obesity, eating disorders, and sleep disorder.

16. The method according to claim 14, wherein the central nervous system disorder is 5-HT6 receptor mediated central nervous system disorder.

17. A method for treating 5-HT6 receptor mediated disorders in a subject comprising administering a pharmaceutically effective amount of the substituted-1H-quinazoline-2,4-dione derivative of claim 1 or a pharmaceutically acceptable salt thereof to the subject in need thereof.
